(19) United States
(12) Patent Application Publication
(10) Pub. No.: US 2025/0319162 A1
(43) Pub. Date: Oct. 16, 2025

(54) METHODS AND MATERIALS FOR TREATING OBESITY

(71) Applicant: Regents of the University of California, Oakland, CA (US)

(72) Inventors: Alan R. Saltiel, San Diego, CA (US); Wenmin Xia, San Diego, CA (US)

(21) Appl. No.: 19/040,461

(22) Filed: Jan. 29, 2025

Related U.S. Application Data
(60) Provisional application No. 63/626,822, filed on Jan. 30, 2024.

(51) Int. Cl. A61K 38/26; A61K 9/00; A61K 31/4162; A61K 31/522; A61K 31/7028; A61K 38/22; A61K 48/00; A61P 1/16; A61P 3/04; C12N 9/22; C12N 15/11

(52) U.S. Cl. CPC A61K 38/26; A61K 9/0019; A61K 31/4162; A61K 31/522; A61K 31/7028; A61K 38/2264; A61K 48/005; A61P 1/16; A61P 3/04; C12N 9/222; C12N 15/111; C12N 2310/20

(57) ABSTRACT

Provided herein are methods and materials for treating or reducing the risk of obesity and/or conditions associated with obesity (e.g., type 2 diabetes or hyperinsulinemia). For example, provided herein are methods and materials for inhibiting RalA to treat obesity and/or conditions associated with obesity in a subject in need thereof. In some cases, the subject has elevated expression of RalA compared to a healthy individual.

### CLAIM OF PRIORITY

This application claims the benefit of U.S. Provisional Application Ser. No. 63/626,822, filed on Jan. 30, 2024. The entire contents of the foregoing are incorporated herein by reference.

### FEDERALLY SPONSORED RESEARCH OR DEVELOPMENT

This invention was made with Government support under DK063491, DK122804, DK124496, DK125820, and DK128796 awarded by the National Institutes of Health. The Government has certain rights in the invention.

### TECHNICAL FIELD

The present disclosure relates to methods and materials for treating obesity and/or conditions associated with obesity. For example, this document relates to inhibiting RalA to treat obesity and/or conditions associated with obesity in a subject in need thereof.

### BACKGROUND

Obesity has become a worldwide epidemic, dramatically increasing the incidence of type 2 diabetes, nonalcoholic steatohepatitis and other cardiometabolic abnormalities. Mitochondrial dysfunction has been associated with obesity, insulin resistance, and fatty liver disease. However, what drives mitochondrial damage and how it contributes to obesity and its many complications remains unknown. Thus, there is an unmet clinical need for improved treatment regimens for patients suffering from obesity and/or conditions associated with obesity.

### SUMMARY

This document provides methods and materials for treating or reducing the risk of obesity or a condition associated with obesity. For example, this document provides methods and materials for using RalA inhibitors (e.g., 6-Amino-1,3-dimethyl-4-(4-(trifluoromethyl)phenyl)-1,4-dihydropyrano[2,3-c]pyrazole-5-carbonitrile, SCH-53239, SCH-54292, BQU57, RBC6, RBC8, and/or RBC10) to treat a subject in need identified as having hyperinsulinemia, type 2 diabetes, prediabetes, and/or fatty liver disease.

As demonstrated herein, inhibiting the expression of RalA in mice attenuated high fat diet-induced obesity by increasing energy expenditure and mitochondrial oxidative phosphorylation in adipocyte tissues (e.g., inguinal white adipocyte tissue). Provided herein methods of treating or reducing the risk of obesity or a condition associated with obesity in a subject in need thereof, the method including administering a therapeutically effective amount of a pharmaceutical composition including an RalA inhibitor. Also provided herein are methods of treating obesity or a condition associated with obesity in a subject in need thereof, the method including: a) identifying the subject as having hyperinsulinemia, type 2 diabetes, prediabetes, a BMI of 30 or greater, a waist-hip ratio of 1.0 or more, and/or fatty liver disease, b) administering a therapeutically effective amount of a pharmaceutical composition including an RalA inhibitor.

In some embodiments, the method further includes administering to the subject a second pharmaceutical composition.

In some embodiments, second pharmaceutical composition is administered before, after, or concurrent with the pharmaceutical composition including the RalA inhibitor.

In some embodiments, concurrent administration of the pharmaceutical composition including the RalA inhibitor, and the second composition is administered as a single composition.

In some embodiments, the second pharmaceutical composition includes naltrexone-bupropion, phentermine-topiramate, orlistat, diethylpropion, setmelanotide, phendimetrazine, benzphetamine, tirzepatide, a glucagon-like peptide-1 receptor (GLP-1) agonist, a glucose-dependent insulinotropic polypeptide (GIP), a GIP antagonist, an amylin agonist, a leptin agonist, and/or a glucagon agonist.

In some embodiments, the GLP-1 agonist includes dulaglutide, exenatide, liraglutide, lixisenatide, and/or semaglutide.

In some cases, the GLP-1 and/or the GIP agonist comprises tirzepatide. In some cases, the amylin agonist includes pramlintide. In some cases, the leptin agonist includes Metreleptin. In some cases, the glucagon agonist includes dasiglucagon, Baqsimi, or Gvoke.

In some embodiments, the condition associated with obesity includes type 2 diabetes, hyperinsulinemia, hepatic steatosis, weight gain, glucose intolerance, heart disease, chronic kidney disease, high cholesterol, gall bladder disease, high blood pressure, sleep apnea, gastroesophageal reflex disease, metabolic syndrome, acute pancreatitis, dyslipidemia, and/or cancer.

In some embodiments, the RalA inhibitor is an inhibitory nucleic acid molecule, a CRISPR/Cas system, or a small molecule inhibitor.

In some embodiments, the RalA inhibitor includes 6-Amino-1,3-dimethyl-4-(4-(trifluoromethyl)phenyl)-1,4-dihydropyrano[2,3-c]pyrazole-5-carbonitrile, SCH-53239, SCH-54292, BQU57, RBC6, RBC8, and/or RBC10.

In some embodiments, the administering includes oral, intravenous, intradermal, intramuscular, and/or subcutaneous administration.

In some embodiments, the subject is a mammal. In some embodiments, the mammal is a human, a monkey, a dog, a cat, a pig, a horse, a cow, a sheep, a goat, a rabbit, a mouse, or a rat.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and figures, and from the claims.

### DESCRIPTION OF DRAWINGS

FIGS. 1A-1K. White adipocyte-specific Rala deletion protected mice from high-fat-diet (HFD) induced obesity. FIG. 1A: Schematic illustrating RalA activation network involving genes encoding RalA, GEF and GAP. FIG. 1B: RNA-seq analysis of primary inguinal (Ing) and epididymal (Epi) mature adipocytes isolated from mice (n=3) under 16-week HFD feeding. Heat map displays transcriptional expression as z-scored FPM values. Adjusted P (adj. P) values are indicated and considered significant with values <0.05. FIG. 1C: Quantification of RalA protein content in mature adipocytes from inguinal WAT (iWAT) and epididymal WAT (eWAT) of mice fed with chow diet (CD) (n=3) or HFD (n=4) for 16 weeks. iWAT P=0.033 CD versus HFD, eWAT P=0.005 CD versus HFD. A.U., arbitrary units. FIG. 1D: Quantification of RalA GTPase activity in iWAT and eWAT of mice (n=4) fed with CD or HFD for 4 weeks. iWAT P=0.0448 CD versus HFD. FIG. 1E: Graph plot showing the difference in the body weight of Rala^{f/f} (n=8) and Rala^{AKO} (n=10) mice fed with 60% HFD over time. Longitudinal graph, P=0.0158, P=0.009, P=0.0106. FIG. 1F: Graph plot showing the difference in the body mass of Rala^{f/f} (n=7) and Rala^{AKO} (n=6) mice fed with HFD for 12 weeks. Fat mass P=0.0252. FIG. 1G: Graph plot showing the difference in fat depot weights of Rala^{f/f} (n=10) and Rala^{AKO} (n=12) mice fed with HFD for 12 weeks. iWAT P=0.0465. FIG. 1H: Graph plot showing the difference in glucose tolerance test (GTT) on 11-week HFD-fed Rala^{f/f} (n=10) and Rala^{AKO} (n=13) mice over time, P=0.0174, P=0.0036, P=0.0069; the area under the curve (AUC) was calculated from longitudinal charts, P=0.0062. FIG. 1I: Graph plot showing the difference in insulin tolerance test (ITT) on 12-week HFD-fed Rala^{f/f} (n=10) and Rala^{AKO} (n=12) mice over time; AUC was calculated from longitudinal chart. FIG. 1J: Graph plot showing the difference in plasma insulin levels in 8-week HFD-fed Rala^{f/f} and Rala^{AKO} mice (n=11). Fasted P=0.0166. Fed P=0.0329. FIG. 1K: HOMA-IR was calculated using fasting glucose and insulin levels from 8-week HFD-fed Rala^{f/f} (n=8) and Rala^{AKO} (n=10) mice. P=0.0152. Data (FIGS. 1C-1K) show mean±s.e.m., *P<0.05, **P<0.01, by two-tailed Student's t-test (FIGS. 1C, 1D, 1F, 1G, 1J, and 1K) or two-way analysis of variance (ANOVA) with Bonferroni's post-test (FIGS. 1E, 1H, and 1I).

FIGS. 2A-2K. Loss of RalA in WAT ameliorated HFD-induced hepatic steatosis. FIG. 2A: A pyruvate tolerance test (PTT) was performed on overnight-fasted Rala^{f/f} (n=7) and Rala^{AKO} (n=5) mice after 8 weeks of HFD feeding; P=0.0093, P=0.0241. The AUC was calculated from a PTT longitudinal chart; P=0.0097. FIG. 2B: Graph plot showing relative mRNA expression of gluconeogenic genes in livers of HFD-fed Rala^{f/f} and Rala^{AKO} mice (n=10). P=0.0388, P=0.0167. FIG. 2C: Graph plot showing liver weight of HFD-fed Rala^{f/f} (n=10) and Rala^{AKO} (n=12) mice; P=0.0235. FIG. 2D: Graph plot showing the levels of triglyceride (TG) in livers of HFD-fed Rala^{f/f} (n=8) and Rala^{AKO} (n=13) mice; P=0.0129. e, Representative H&E staining image (left) and Oil-Red-O staining image (right) of liver sections in HFD-fed Rala^{f/f} and Rala^{AKO} mice (n=3). Scale bar, 15 mm. FIG. 2F: Graph plot showing relative mRNA expression of lipogenic genes in livers of HFD-fed Rala^{f/f} (n=9) and Rala^{AKO} (n=10) mice; P=0.0218, P=0.0435, P=0.0332, P=0.0325. FIG. 2G Plasma leptin levels in HFD-fed Rala^{f/f} (n=7) and Rala^{AKO} (n=6) mice. FIG. 2H: Graph plot showing relative mRNA expression of FAO-related genes in livers of HFD-fed Rala^{f/f} (n=10) and Rala^{AKO} (n=11) mice. FIG. 2I: Graph plot showing relative mRNA expression of genes related to inflammation and fibrosis in livers of HFD-fed Rala^{f/f} (n=7) and Rala^{AKO} (n=11) mice; P=0.0347, P=0.0325. FIGS. 2J and 2K: Graph plot showing plasma AST (FIG. 2J) and ALT (FIG. 2K) activities in HFD-fed Rala^{f/f} (n=7) and Rala^{AKO} (n=14) mice; P=0.0367 (FIG. 2J), P=0.0275 (FIG. 2K). Data (FIGS. 2A-2D and 2F-2K) show mean±s.e.m., *P<0.05, **P<0.01 by two-tailed Student's t-test (FIGS. 2B-2D, 2F, and 2I-2K) or two-way ANOVA with Bonferroni's post-test (FIG. 2A).

FIGS. 3A-3F. RalA deficiency in WAT increased energy expenditure and mitochondrial oxidative phosphorylation. FIG. 3A: Regression plot of energy expenditure (EE) measured in HFD-fed Rala^{f/f} (n=8) and Rala^{AKO} (n=5) mice during dark phase. ANCOVA was performed using body weight (BW) as a covariate, group effect P=0.0391. FIGS. 3B and 3C: Immunoblot (FIG. 3B) and quantification (FIG. 3C) of OXPHOS complex proteins and β-tubulin in iWAT of HFD-fed Rala^{f/f} (n=10) and Rala^{AKO} (n=13) mice. P=0.0005, P=0.0348, P<0.0001. FIGS. 3D and 3E: Plasma non-esterified fatty acid (NEFA; FIG. 3D) and TG (FIG. 3E) levels in HFD-fed Rala^{f/f} (n=10) and Rala^{AKO} (n=13) mice; P=0.0077 (FIG. 3D), P=0.0115 (FIG. 3E). FIG. 3F: Basal OCR in mitochondria measured by Seahorse. Mitochondrial fractions were isolated from primary mature adipocytes in iWAT or eWAT of HFD-fed Rala^{f/f} (n=4) and Rala^{AKO} (n=5) mice. iWAT P=0.0004. Data (FIGS. 3C-3F) show mean±s.e.m., *P<0.05, **P<0.01, ***P<0.001 by two-tailed Student's t-test (FIGS. 3C-3F).

FIGS. 4A-4G. Rala knockout in white adipocytes increased mitochondrial activity and fatty acid oxidation via preventing obesity-induced mitochondrial fission in iWAT. FIG. 4A: OCR was measured in fully differentiated primary adipocytes (n=8 biological samples); P=0.0499, P<0.0001, P<0.0001, P=0.0006, P=0.0468. Vertical arrows indicate injection ports of indicated chemicals. FIG. 4B: 14C-PA oxidation in differentiated primary WT (n=4 biological samples) and KO (n=3 biological samples) adipocytes under basal conditions; P=0.0037. FIG. 4C: Representative confocal images of live primary and immortalized adipocytes stained with TMRM and BODIPY (n=3 biological samples). Scale bar, 15 μm. FIG. 4D: Representative transmission electron microscope (TEM) images of iWAT from CD-fed and HFD-fed Rala^{f/f} and Rala^{AKO} mice (n=3 biological samples). The arrow in HFD-fed Rala^{f/f} panel indicates fissed mitochondria; the arrow in the HFD-fed Rala^{AKO} indicates elongated mitochondria. Scale bar, 1 μm (CD) or 500 nm (HFD). FIG. 4E: Representative TEM images of WT and Rala KO immortalized adipocytes (n=3 biological samples). The arrows indicate elongated mitochondria; asterisk indicates lipid droplet. Scale bar, 2 μm. FIGS. 4F and 4G: Histogram (FIG. 4F) and violin plot (FIG. 4G) of maximal mitochondrial length in immortalized adipocytes (WT, six independent cells; KO, ten independent cells). Violin plot is presented as violin showing 25th to 75th percentiles and whiskers showing min to max; P=0.047 (FIG. 4F), P<0.0001 (FIG. 4G). Data (FIGS. 4A and 4B) show mean #s.e.m., *P<0.05, ***P<0.001, ****P<0.0001 by two-tailed Student's t-test (b,g), two-way ANOVA alone (FIG. 4F) or with Bonferroni's post-test (FIG. 4A).

FIGS. 5A-5J. Inhibition of RalA increased Drp1 S637 phosphorylation in white adipocytes. FIG. 5A: Quantification of phospho-Drp1 (S637) and total Drp1 in iWAT of HFD-fed Rala^{f/f} (n=10) and Rala^{AKO} (n=13) mice; P=0.0001. FIGS. 5B and 5C: Immunoblotting (FIG. 5B) and quantification (FIG. 5C) of phospho-Drp1 (S637) and total Drp1 in immortalized adipocytes (n=4 biological samples); P=0.0125 (FIG. 5C). Adipocytes were treated with 20 μM forskolin (Fsk) for indicated time. FIGS. 5D and 5E: Immunoblotting (FIG. 5D) and quantification of phospho-Drp1 (S637) and total Drp1 in human primary adipocytes (SGBS) (n=4 biological samples). Cells were pretreated with 50 μM RBC8 or dimethylsulfoxide (DMSO) for 30 min before treatment with 20 μM forskolin (Fsk) for indicated time; P=0.0022, P=0.0244 (FIG. 5E). FIG. 5F: Basal 14C-PA oxidation in WT immortalized adipocytes transfected with indicated plasmids (n=6 biological samples); P=0.040 3.1 versus Drp1^{WT}, P=0.0364 Drp1^{WT} versus Drp1^{SD}. FIG. 5G: Representative TEM images of immortalized adipocytes transfected with indicated plasmids (n=3 biological samples). Scale bar, 2 μM. FIGS. 5H and 51: DNM1L mRNA expression is correlated with BMI (FIG. 5H) and HOMA (FIG. 5I) in human abdominal subcutaneous adipose tissue samples (n=56 biological samples). p (rho) denotes Spearman's rank-order correlation coefficient of the regression; P=0.024 (FIG. 5H), P=0.024 (FIG. 5I). FIG. 5J: Box-and-whisker plot of DNM1L mRNA expression in abdominal subcutaneous adipose tissues from 56 individuals with or without obesity. Benjamini-Hochberg adj. P=0.014. The box plot is presented as a box showing 25th to 75th percentiles and whiskers showing min to max. Data (FIGS. 5A, 5C, 5E, and 5F) show mean±s.e.m., *P<0.05, **P<0.01, ***P<0.001 by two-tailed Student's/-test (FIGS. 5A, 5C, 5E, and 5F). Significance in correlation was assessed by Spearman's correlation test (FIGS. 5H, and 5J).

FIGS. 6A-6J. RalA interacted with Drp1 and protein phosphatase 2A, promoting dephosphorylation of Drp1 at S637. FIG. 6A: Representative immunoblotting of pulldown assay determining PP2Aa-RalA interactions. FIG. 6B: Representative immunoblotting of co-immunoprecipitation (co-IP) determining the interaction between RalA WT, constitutive active (G23V) or dominant negative (S28N) mutants and PP2Aa in HEK293T cells. FIG. 6C: Representative immunoblotting of pulldown and in vitro loading assay determining interaction between PP2Aa and GTP/GDP-loaded RalA. Purified Flag-RalA^{WT} protein loaded with either GTPγS or GDP was, respectively, used as a bait to pull down GFP-PP2Aa from HEK293T cells. FIG. 6D: Representative immunoblotting of in vitro dephosphorylation assay in HEK293T cells co-transfected with PP2A and Drp1 plasmids. Cells were treated for 1 h with 20 μM forskolin (Fsk) or vehicle. FIG. 6E: Representative immunofluorescent staining of endogenous Drp1 and RalA in immortalized WT adipocytes. Scale bar, 5 μm. FIG. 6F: Representative immunoblotting of RalA activity assay in immortalized Rala KO adipocytes reconstituted with RalA^{WT} and RalA^{G23V}. FIG. 6G: Immunoblotting of phospho-Drp1 (S637), total Drp1, Flag-tagged RalA and β-actin in immortalized Rala KO adipocytes with or without RalA reconstitution (n=3 independent experiments). Adipocytes were treated with 20 μM forskolin for the indicated times. FIG. 6H: Representative confocal images of live immortalized adipocytes (n=3 biological independent cells) stained with TMRM (red) and BODIPY (green). Scale bar, 15 μM. FIG. 6I: OCR was measured by Seahorse in immortalized adipocytes (KO, n=5 independent samples; +WT, n=10 independent samples; +G23V, n=9 independent samples); P=0.0165 KO versus +WT, P=0.0005 KO versus +G23V. Vertical arrows indicate injection ports of indicated chemicals. Data are shown as mean±s.e.m., *P<0.05, ***P<0.001 by two-way ANOVA. FIG. 6J: Representative TEM images of Rala KO immortalized adipocytes with or without RalA reconstitution (n=3 independent cells). Blue arrow indicates elongated mitochondria; asterisk indicates lipid droplet. Scale bar, 2 μm.

FIGS. 7A-7P. RalA protein content and activity increased in obese adipocytes. FIG. 7A: Immunoblotting for RalA in mature adipocytes isolated from iWAT (n=3) or eWAT (n=4) of age-matched CD-fed (lean) mice and HFD-fed (obese) mice. FIG. 7B: Representative immunoblotting for active RalA (aRalA) in iWAT (upper panel) or eWAT (lower panel) of age-matched CD-fed mice (n=3) and HFD-fed mice (n=2). FIG. 7C: RalA mRNA expression and quantified protein levels in BAT of age-matched CD-fed and HFD-fed mice (n=8). FIG. 7D: Immunoblotting of RalA in inguinal mature adipocyte fraction, eWAT, BAT, and liver from lean mice (n=3). FIG. 7E: In vivo and in vitro activation of RalA by insulin in adipose tissue of Rala^{f/f} (saline n=3, insulin n=2) and Rala^{AKO} (n=4) mice and primary adipocytes. 0.5 U/kg or 100 nM insulin was injected or treated for 5 min or indicated time. FIG. 7F: Basal in vivo glucose uptake in 6 hrs fasted CD-fed mice injected with 10 μCi [¹⁴C]-2deoxyglucose for 30 min (Rala^{f/f} n=7, Rala^{AKO} n=5). FIG. 7G: Plasma insulin levels before and 30 min after glucose injection (Rala^{f/f} n=9, Rala KO n=5). FIG. 7H: Insulin stimulated in vivo glucose uptake in CD-fed mice injected with 1.2 g/kg glucose and 10 μCi [³H]-2-deoxy-glucose for 30 min (Rala^{f/f} n=6, Rala^{AKO} n=9). FIG. 7I: Immunoblotting of RalA in BAT (upper panel) and eWAT (lower panel) of Rala^{f/f} and Rala^{BKO} mice (n=3). FIG. 7J: Basal in vivo glucose uptake in 6 hrs fasted CD-fed mice injected with 10 μCi [¹⁴C]-2-deoxy-glucose for 30 min (Rala^{f/f} n=7, Rala^{BKO} n=5). FIG. 7K: Plasma insulin levels before and 30 min after glucose injection (Rala^{f/f} n=5, Rala^{BKO} n=7). FIG. 7L: Insulin stimulated in vivo glucose uptake in CD-fed mice injected with 1.2 g/kg glucose and 10 μCi [³H]-2-deoxy-glucose for 30 minutes (Rala^{f/f} n=5, Rala^{BKO} n=7). FIG. 7M: Representative immunostaining of endogenous RalA and GLUT4 in primary adipocytes treated with insulin (100 nM) or vehicle for 30 min, scale bar=15 μm. (n=3 biological samples). FIG. 7N: Representative immunoblotting of RalA, GLUT4, IRAP and Na+/K+—ATPase proteins in plasma membrane fraction of primary adipocytes treated with vehicle or insulin (100 nM) for 30 min. (n=3 biological samples). FIG. 7O: 2-deoxy-glucose (2-DG) uptake in primary adipocytes treated with insulin (100 nM) or vehicle for 30 min (n=3 biological samples). FIG. 7P: Immunoblotting of phosphor-Akt (S473), total Akt and GAPDH in primary adipocytes treated with or without insulin (100 nM) for 15 min. (n=3 biological samples). The data are shown as the mean±SEM, *P<0.05, **P<0.01 by two-tailed Student's T-test (FIGS. 7H, 7L, and 70).

FIGS. 8A-8S. Brown adipocyte specific Rala deletion in mice did not phenocopy Rala^{AKO} mice. FIGS. 8A-8C: Body weight curve (FIG. 8A), body composition (FIG. 8B) and fat depot weights (FIG. 8C) of CD-fed Rala^{f/f} (n=8) and Rala^{AKO} (n=12) mice at the age of 24-weeks. P=0.0103 (FIG. 8B). P=0.0122, P=0.0252, P=0.0403 (FIG. 8C). FIG. 8D: Representative H&E staining images of iWAT, eWAT, and BAT from CD-fed and HFD-fed mice (n=3), scale bar=100 μm, representative adipocytes size quantification of iWAT from CD-fed and HFD-fed mice. FIGS. 8D and 8F: Glucose tolerance test (GTT, FIG. 8E) and insulin tolerance test (ITT, FIG. 8F) on CD-fed Rala^{f/f} (n=8) and Rala^{AKO} (n=12) mice. Area under curves (AUC) were calculated from GTT and ITT, respectively. P=0.0247 (FIG. 8F). FIG. 8G: Plasma insulin levels in CD-fed Rala^{f/f} and Rala^{AKO} mice under ab libitum (n=5) or overnight fasted (n=9) condition. FIG. 8H: Homeostasis model assessment-estimated insulin resistance (HOMA-IR) was calculated based on fasting glucose and insulin levels from CD-fed Rala^{f/f} and Rala^{AKO} mice (n=9). FIG. 8I: Blood glucose levels in CD-fed mice (n=5) at indicated states. FIG. 8J: Blood glucose levels in HFD-fed Rala^{f/f} (n=9) and Rala^{AKO} (n=11) mice at indicated states. FIGS. 8K and 8L: Body composition (FIG. 8K) and fat depot weights (FIG. 8I) in CD-fed Rala^{f/f} (n=9) and Rala^{BKO} (n=8) mice at the age of 28 weeks. P=0.0488 (FIG. 8L). FIGS. 8M and 8N: GTT (FIG. 8M) and ITT (FIG. 8N) were performed in CD-fed Rala^{f/f} (n=9) and Rala^{BKO} (n=8) mice at the age of 26-weeks. FIG. 8O: Body weight curve of Rala^{f/f} (n=6) and Rala^{BKO} (n=4) mice fed with HFD for 12 weeks. FIGS. 8P and 8Q: Body composition (FIG. 8P) and fat depot and liver weights (FIG. 8Q) in HFD-fed Rala^{f/f} (n=7) and Rala^{BKO} (n=5) mice at the age of 20-21 weeks. FIGS. 8R and 8S: GTT (FIG. 8R) and ITT (FIG. 8S) were performed in HFD-fed Rala^{f/f} (n=7) and Rala^{BKO} (n=5) mice at the age of 17-weeks or 20-weeks, respectively. The data are presented as the mean±SEM (FIGS. 8A-C and 8E-8S), *P<0.05 by two-tailed Student's T-test (FIGS. 8B, 8C, 8F, and 8L) or two-way ANOVA with Bonferroni's post-test (FIGS. 8A, 8E, 8F, 8M-80, and 8R, and 8S).

FIGS. 9A-9S. Neither CD-fed RalaAKO mice nor HFD-fed RalaBKO mice showed increased energy expenditure. FIG. 9A: Regression plot of energy expenditure (EE) during dark phase against body weight (BW) in CD-fed Rala^{f/f} and Rala^{AKO} mice (n=8). ANCOVA test using BW as a covariate, group effect P=0.2805. FIGS. 9B-9E: BW-normalized oxygen consumption (FIG. 9B), respiratory exchange ratio (RER) (FIG. 9C), pedestrian locomotion (FIG. 9D) and food intake (FIG. 9E) over a two-days period were measured in CD-fed Rala^{f/f} and Rala^{AKO} mice (n=8) by metabolic cages. FIGS. 9F-9I: BW-normalized oxygen consumption (FIG. 9F), respiratory exchange ratio (RER) (FIG. 9G), pedestrian locomotion (FIG. 9H) and food intake (FIG. 9I) over a two-days period were measured in HFD-fed Rala^{f/f} (n=8) and Rala^{AKO} (n=5) mice by metabolic cages. FIG. 9J: Regression plot of EE during dark phase against BW in HFD-fed Rala^{f/f} (n=7) and Rala^{BKO} (n=5) mice. ANCOVA test using BW as a covariate, group effect P=0.2792. FIGS. 9K-9N: BW-normalized oxygen consumption (FIG. 9K), respiratory exchange ratio (RER, FIG. 9L), pedestrian locomotion (FIG. 9M) and food intake (FIG. 9N) over a two-days period were measured in HFD-fed Rala^{f/f} (n=7) and Rala^{BKO} (n=5) mice by metabolic cages. (FIGS. 9O and 9P) Immunoblotting (FIG. 9O) and quantification (FIG. 9P) of OXPHOS proteins in eWAT of HFD-fed mice (Rala^{f/f} n=8 and Rala^{AKO} n=12). (FIGS. 9Q and 9R) Immunoblotting (FIG. 9Q) and quantitation (FIG. 9R) of OXPHOS proteins in BAT of HFD-fed mice (Rala^{f/f} n=10 and Rala^{f/f} n=13). P<0.0001, P=0.0252 (FIG. 9R). FIG. 9S: Relative mRNA expression of browning-related genes in iWAT, eWAT and BAT of HFD-fed mice (Rala^{f/f} n=8 and Rala^{AKO} n=13). P<0.0001, P<0.0001, P<0.0001. The data (FIGS. 9B-81, 9K-9N, and 9P-9S) are shown as the mean±SEM, *P<0.05, ***P<0.001 by two-tailed Student's T-test (FIGS. 9D, 9E, 9H, 9I, 9M, 9N, and 9P-9S), or two-way ANOVA with Bonferroni's post-test (FIGS. 9B, 9C, 9F, 9G, 9K, and 9L).

FIGS. 10A-10J. Absence of RalA in adipocytes did not affect free fatty acid release. FIG. 10A: Calculation of individual OCR in differentiated primary adipocytes (n=8 biological samples). P=0.0008, P=0.0175, P=0.0042. FIG. 10B: Representative immunoblot of RalA and β-Tubulin in immortalized preadipocytes and differentiated adipocytes. (n=3 biological samples). FIG. 10C: Quantification of mean TMRM fluorescence intensity in primary adipocytes and immortalized adipocytes (n=3 independent cells). P<0.0001, P<0.0001. FIG. 10D: Time course TMRM intensity quantification in primary and immortalized adipocytes (n=4 biological samples). Adipocytes were treated with 1 μM CL-316,243 (CL) for indicated times. P=0.0015, P=0.0044 (left panel). P<0.0001, P=0.0154, P=0.0095 (right panel). FIGS. 10E and 10F: Quantification of NEFA (FIG. 10E) and free glycerol (FIG. 10F) released into medium from immortalized adipocytes (n=3 biological samples). Cells were treated with 1 μM CL, 100 nM insulin, or in combination prior to medium collection. P=0.0301, P=0.0051 (FIG. 10F). FIGS. 10G and 10H: Plasma levels of NEFA (FIG. 10G) and free glycerol (FIG. 10H) in CD-fed Rala^{f/f} and Rala^{AKO} mice (n=6). P=0.0027, P=0.0173 (FIG. 10H). Mice were i.p. injected with CL or vehicle prior to blood sampling. FIGS. 10I and 10J: Plasma levels of NEFA (FIG. 10I) and free glycerol (FIG. 10J) in CD-fed Rala^{f/f} (n=4) and Rala^{AKO} (n=5) mice. Mice were either subjected to ad libitum feeding, overnight fasting or fasting plus insulin injection 15 min prior to harvesting. P=0.0316 (FIG. 10J). The data (FIGS. 10A and 10C-10J) are shown as the mean±SEM, *P<0.05, **P<0.01, ***P<0.001, ****P<0.0001 by two-tailed Student's T-test (FIGS. 10A, 10C, 10E-10J) or two-way ANOVA with Bonferroni's post-test (FIG. 10D).

FIGS. 11A-11O: RalA inhibition did not affect mitochondrial biogenesis in WAT. FIGS. 11A and 11B: Relative mRNA expression of genes corresponding to mitochondrial biogenesis in iWAT (FIG. 11A) and eWAT (FIG. 11B) of HFD-fed Rala^{f/f} (n=9) and Rala^{AKO} (n=10) mice. P=0.0449, P=0.0478. (FIG. 11A). FIGS. 11C-11F: Immunoblotting (FIGS. 11C-11D) and quantification (FIGS. 11E-11F) of phospho-AMPK (T172), total AMPK and β-Tubulin in iWAT (Rala^{f/f} n=9, Rala^{AKO} n=14) (FIGS. 11C-11E) and eWAT (Rala^{f/f} n=8, Rala^{AKO} n=12) (FIGS. 11D-11F) of HFD-fed mice. FIG. 11G: Maximal mitochondrial length in iWAT of CD-fed and HFD-fed mice (n=3 biological samples). P<0.0001, P<0.0001, P=0.1675. FIG. 11H: Representative TEM images of mitochondria in eWAT of HFD-fed mice, scale bar=500 nm. FIG. 11I: Representative TEM images of mitochondria in BAT of HFD-fed mice, scale bar=1 μm. FIGS. 11J-11L: Immunoblotting (FIG. 11J) and quantification (FIGS. 11K and 11L) of Opa1 in iWAT of HFD-fed mice (Rala^{f/f} n=10, Rala^{AKO} n=13). P=0.0045 (FIG. 11K). P=0.0044 (FIG. 11L). m-o, Immunoblotting (FIG. 11M) and quantification (FIGS. 11N and 110) of Opa1 in eWAT of HFD-fed mice (Rala^{f/f} n=14, Rala^{AKO} n=10). P=0.0063 (FIG. 11O). The data (FIGS. 11A, 11B, 11E-11G, 11K, 11L, 11N, and 11O) are shown as the mean±SEM, *P<0.05, **P<0.01, ****P<0.0001 by two-tailed Student's T-test (FIGS. 11A, 11G, 11K, 11L, and 11O).

FIGS. 12A-12R: Rala deletion in adipocytes did not affect cAMP production and HSL phosphorylation. Immunoblotting of phospho-Drp1 S637, total Drp1 and β-Tubulin in iWAT of HFD-fed Rala^{f/f} (n=10) and Rala^{AKO} (n=13) mice. FIGS. 12B and 12C: Immunoblotting (FIG. 12B) and quantification (FIG. 12C) of phospho-Drp1(S637), total Drp1, and β-Tubulin in eWAT of HFD-fed Rala^{f/f} (n=8) and Rala^{AKO} (n=12) mice. FIGS. 12D and 12E: Immunoblotting (FIG. 12D) and quantification (FIG. 12E) of phospho-Drp1(S637), total Drp1 and β-Actin in iWAT of CD-fed mice. Non-fasted Rala^{f/f} (vehicle n=3, CL n=4) and Rala^{AKO} (vehicle n=3, CL n=5) mice fed with CD were i.p. injected with 1 mg/kg CL for 30 min. P=0.0054, P=0.0135 (FIG. 12E). FIGS. 12F and 12G: Immunoblotting (FIG. 12F) and quantification (FIG. 12G) of phospho-Drp1(S637), total Drp1, RalA, and β-Actin in fully differentiated primary adipocytes (n=4 biological samples). P=0.0085, P=0.0046 (FIG. 12G). Adipocytes were differentiated from stromal vascular fraction (SVF) isolated from 8-week-old female mice, and were treated with 1 μM CL for indicated time. FIGS. 12H and 12I: Immunoblotting (FIG. 12H) and quantification (FIG. 12I) of phospho-Drp1(S637), total Drp1, RalA, and β-Actin in immortalized adipocytes (n=4 biological samples). P=0.0159, P=0.0086 (FIG. 12I). Adipocytes were treated with 1 μM CL for indicated time. FIGS. 12J and 12K: Immunoblotting (FIG. 12J) and quantification (FIG. 12K) of phospho-Drp1(S637), total Drp1, and β-Actin in 3T3-L1 adipocytes (n=4 biological samples). P=0.0009 (FIG. 12K). Cells were pretreated with 50 μM RBC8 or DMSO for 30 min, then treated with 5 μM forskolin (Fsk) for indicated time. FIG. 12L: Determination of intracellular cAMP levels in differentiated WT and Rala^{AKO} (KO) primary adipocytes (n=4 biological samples). Cells were treated with CL for 5 min prior to harvesting. FIGS. 12M and 12N: Quantification of phospho-HSL (S660) and HSL in primary (FIG. 12M) and immortalized adipocytes (FIG. 12N) treated with CL for indicated time (n=4 biological samples). FIG. 12O: Maximal mitochondrial length in immortalized adipocytes expressing Drp1 mutants (n=3 independent cells). P<0.0001 3.1 vs Drp1^{WT}, P<0.0001 Drp1^{WT} vs Drp1^{SD}, P<0.0001 Drp1^{SD} vs Drp1^{SA}. FIGS. 12P-12R: Transcriptomics and clinical data were directly accessed from GEO database (GSE70353). FIGS. 12P and 12Q: DNM1L. mRNA expression is correlated with BMI (FIG. 12P) and HOMA (FIG. 12Q) in human subcutaneous adipose tissue samples (n=770). P=0.0002 (FIG. 12P). P=0.0019 (FIG. 12Q). FIG. 12R: Box-and-whisker plot of DNM1L, mRNA expression in abdominal subcutaneous adipose tissues from 770 individuals with or without obesity. Benjamini and Hochberg-adjusted P value (adj. p) is 0.014186. The box plot is presented as a box: 25^{th} to 75^{th} percentile, and whiskers: min to max. The data (FIGS. 12C, 12E, 12G, 12I, 12K-12O) are shown as the mean±SEM, *P<0.05, **P<0.01, ***P<0.001, ****P<0.0001 by two-tailed Student's T-test (FIGS. 12E, 12G, 12I, 12K, and 12O). Significance in correlation was assessed by Spearman's correlation test (FIGS. 12P and 12Q).

FIGS. 13A-13K. Knockout of RalA increased PP2Aa content. FIG. 13A: Representative immunoblotting of co-immunoprecipitated between Flag-RalA^{WT}, Flag-RalA^{G23V}, or Flag-RalA^{S28N} and Myc-Drp1 proteins in HEK293T cells. FIG. 13B: Representative in vitro dephosphorylation assay in PP2Ab and Drp1 co-transfected HEK293T cells treated with or without 20 μM forskolin (Fsk) for 1 hr. FIG. 13C: Quantification of Drp1 and RalA co-localization using Pearson's method (n=3 independent cells). P<0.0001. FIG. 13D: Quantification of phospho-Drp1 (S637) and total Drp1 in immortalized RalA KO adipocytes with or without RalA reconstitution (n=3 biological samples). 15 min: P=0.0164 KO vs +WT, P=0.0015 KO vs +GV. 30 min: P=0.0185 KO vs +WT, P=0.0087 KO vs +GV. Adipocytes were treated with 20 μM forskolin for indicated time. FIG. 13E: Quantification of TMRM fluorescence intensity in immortalized RalA KO adipocytes with or without RalA reconstitution (n=3 independent cells). P<0.0001 KO vs +WT. P<0.0001 KO vs +GV. FIG. 13F: Calculated OCR in each state from immortalized RalA KO adipocytes with or without RalA reconstitution (WT=6, +WT=10, +GV=9 biological samples). P<0.0001, P=0.0423, P=0.0015, P=0.0060. FIG. 13G: Maximal mitochondrial length in immortalized adipocytes (n=3 independent cells). P<0.0001 KO vs +WT. P<0.0001 KO vs +GV. FIGS. 13H and 13I: Quantification of fission (FIG. 13H) and fusion (i) events in immortalized adipocytes (KO=92, +WT=41). P=0.0252 (FIG. 13H). P=0.0879 (FIG. 13I). FIGS. 13J and 13K: Immunoblotting (FIG. 13J) and quantification (FIG. 13K) of PP2Aa, PP2Ab, PP2Ac, and β-Tubulin in iWAT of HFD-fed mice (Ralaf/f n=10, RalaAKO n=13). P=0.0039 (FIG. 13K). The data (c, d, e, f, g, k) are shown as the mean±SEM, *P<0.05, **P<0.01, ***P<0.001, ****P<0.0001 by two-tailed Student's T-test (FIGS. 13C-13H and 13K).

FIG. 14. Mechanistic model depicting how RalA regulates mitochondrial function in obese adipocytes. Obesity drives RalA expression and GTP binding activity, leading to its association with PP2Aa, which in turn recruits the catalytic subunit PP2Ac to dephosphorylate Drp1 S637. Also, catecholamine resistance could reduce PKA-catalyzed S637 phosphorylation. The combined effects converging on RalA-PP2A-Drp1signaling axis result in constitutive mitochondrial translocation of Drp1 and fragmented mitochondria in adipocytes from obese subjects.

FIG. 15. Schematic depicting the structure of RalA inhibitor BQU57.

FIGS. 16A-16C. RalA inhibitor, BQU57 reduced body weight in mice. FIG. 16A: Bar plot showing body weight of mice before the treatment. FIG. 16B: Longitudinal graph showing changes in the body weight of mice treated with either BQU57 or vehicle (control). FIG. 16C: Bar plots showing changes in body weight after 23 days of treatment.

FIG. 17. Bar plot showing changes in tissue weight post treatment with BQU57.

FIGS. 18A and 18B. FIG. 18A: Graph plot showing changes in the blood glucose level in mice treated with either BQU57 or vehicle. FIG. 18B: Bar plot showing changes in the blood glucose level at endpoint.

### DETAILED DESCRIPTION

Obesity has become a worldwide epidemic, dramatically increasing the incidence of type 2 diabetes, nonalcoholic steatohepatitis and other cardiometabolic abnormalities. During the development of obesity, white adipose tissue (WAT) chronically expands and undergoes metabolic changes characterized by hormone insensitivity, inflammation, fibrosis and apoptosis. While mitochondria play an important metabolic role in healthy adipocytes, oxidizing fuel to produce ATP and generating heat during thermogenesis, mitochondrial function is impaired in obese individuals; however, what drives mitochondrial damage and how it contributes to obesity and its many complications remains unknown.

Provided herein are the foundational discoveries that chronic activation of RalA represses energy expenditure in obese adipose tissue by shifting the balance of mitochondrial dynamics toward excessive fission, contributing to weight gain and metabolic dysfunction. The present methods show that targeted deletion of RalA in white adipocytes can prevent mitochondrial fragmentation and diminishes high fat diet (HDF)-induced weight gain by increasing fatty acid oxidation. Further, the beneficial effects of RalA deletion are driven by a reversal of the increased mitochondrial fission in white adipocytes induced by HDF.

In summary, the present methods demonstrate the role of RalA in mitochondrial dysfunction, a characteristic trait of obesity in humans and rodent adipocytes. Thus, described herein are methods and materials for inhibiting RalA to treat or reduce the risk of obesity or a condition associated with obesity.

### Mitochondrial Damage in Obesity

Obesity is associated with hyperinsulinemia and diabetes and studies have suggested a link between mitochondrial dysfunction, reduced energy expenditure, and insulin resistance. Mitochondria play a pivotal role in generating cellular energy and regulating various metabolic processes. Mitochondrial damage or dysfunction is implicated as a contributing factor in various chronic diseases, including obesity and insulin resistance/type 2 diabetes. Altered mitochondrial oxidative function has been observed in muscle as well as adipose tissue from obese individual compared to healthy weight individuals and adipocytes from obese individuals contain fewer mitochondria compared to lean counterparts. Moreover, the mitochondria in the muscle of obese individuals are fragmented. Changes in mitochondrial size and number are controlled by the dynamic balance of fusion and fission. Fusion is crucial for the optimal control of mitochondrial number and integrity, particularly in response to changes in energy needs. Fission, which is catalyzed by the dynamin-related protein Drp1, mediates mitochondrial division and quality control during cell division; however, mitochondrial fusion and fission are observed in many nondividing cells, indicating that the correct balance of these processes helps adapt to energy needs and ensures homeostasis.

### RalA

Ral GTPases are members of the Ras superfamily involved in multiple cellular processes. RalA is activated by insulin in adipocytes and in turn interacts with members of the exocyst complex to target GLUT4 vesicles to the plasma membrane for docking and subsequent fusion, leading to increased glucose uptake²¹⁻²³. Insulin activates RalA through inhibitory phosphorylation of the RalGAP complex²⁴, as well as localization of RGL2, a guanine-nucleotide exchange factor (GEF) for RalA²⁵. RalA suppresses mitochondrial oxidative function in adipocytes by increasing fission through reversing the inhibitory phosphorylation of the mitochondrial fission protein Drp1. This reduced phosphorylation results from the recruitment of the regulatory subunit of PP2A, which acts as a bona fide effector of RalA, leading to the specific dephosphorylation of the inhibitory Ser637 residue on Drp1, rendering the protein active. Chronic elevation in RalA activity represses energy expenditure in obese adipose tissue, causing weight gain and related metabolic dysfunction, such as glucose intolerance and fatty liver, and may explain in part how energy expenditure is repressed in prolonged obesity. Also, persistent elevation of RalA in obesity produces mitochondrial dysfunction in white adipocytes, with profound effects on systemic metabolism.

As demonstrated herein, a HDF causes mitochondrial fragmentation in inguinal white adipocytes (iWAT), leading to reduced oxidative capacity by a process dependent on the small GTPase RalA. Moreover, RalA expression and activity are increased in white adipocytes after HFD. Thus, prolonged activation of the small GTPase RalA is involved in controlling mitochondrial morphology and function in the context of obesity. The findings of the present disclosure show that RalA is both induced and activated in white adipocytes after HFD feeding, whereas the negative regulator of RalA, RalGAP, is downregulated. Thus, the present disclosure demonstrates that RalA gene and protein expression and activity are increased in adipocytes from obese mice and further that targeted deletion of Rala in white, but not brown, adipocytes attenuates HFD-induced obesity, due to dramatically increased energy expenditure and mitochondrial oxidative phosphorylation, specifically in iWAT.

### RalA Inhibitors

Provided herein are RalA inhibitors that can be used to treat obesity or reduce the risk of obesity or a condition associated with obesity in a subject. In some embodiments, an RalA inhibitor can reduce and/or inhibit the activity or expression of RalA (e.g., expression of RalA transcript and/or expression of RalA polypeptide). In some embodiments, RalA inhibitor that can reduce and/or inhibit the expression of RalA can be an inhibitory nucleic acid molecule, a gene editing molecule, or a small molecule inhibitor.

In some embodiments, an inhibitory nucleic acid molecule is an antisense oligonucleotide, a shRNA, a siRNA, or a microRNA that can bind to RalA transcript and inhibit and/or reduce its expression and/or activity. An inhibitory nucleic acid molecule can include a nucleic acid sequence that is from about 8 to about 80 nucleotides in length (e.g., from about 13 nucleotides to about 80 nucleotides, from about 12 nucleotides to about 50 nucleotides, from about 12 nucleotides to about 30 nucleotides, from about 15 nucleotides to about 30 nucleotides, from about 20 nucleotides to about 30 nucleotides, from about 20 nucleotides to about 24 nucleotides, or from about 16 nucleotides to about 20 nucleotides). In some cases, an inhibitory nucleic acid molecule can include a nucleic acid sequence that is about 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, or 80 nucleotides in length. Examples of RalA transcripts and polypeptides that can be targeted include, without limitation, at least a portion of those set forth in the National Center for Biotechnology Information (NCBI) databases as shown in Table 1.

TABLE 1
Human RalA gene transcript and polypeptide sequences

| Gene | Transcript Accession | Polypeptide Accession |
|---|---|---|
| RalA | XM_047420682.1 | XP_047276638.1 |
| RalA | XM_047420681.1 | XP_047276637.1 |
| RalA | XM_054358752.1 | XP_054214727.1 |
| RalA | XM_054358751.1 | XP_054214726.1 |

In some embodiments, a clustered regularly interspaced short palindromic repeat (CRISPR)/Cas system can be used (e.g., can be introduced into a cell) to reduce and/or inhibit the activity or expression of RalA. Some CRISPR/Cas systems preferentially edit DNA, whereas other CRISPR/Cas systems preferentially modulate RNA. Examples of CRISPR/Cas systems that can be used in the methods described herein are described in Burmistrz M, Krakowski K, Krawczyk-Balska A. RNA-Targeting CRISPR-Cas Systems and Their Applications. Int J Mol Sci. 2020 Feb. 7; 21(3): 1122. doi: 10.3390/ijms21031122; Louise Bendixen, Trine I. Jensen, Rasmus O. Bak, CRISPR-Cas-mediated transcriptional modulation: The therapeutic promises of CRISPRa and CRISPRi, Molecular Therapy, Volume 31, Issue 7, 2023, Pages 1920-1937, ISSN 1525-0016, doi.org/10.1016/j.ymthe.2023.03.024; Hillary, V. E., Ceasar, S.A. A Review on the Mechanism and Applications of CRISPR/Cas9/Cas12/Cas13/Cas14 Proteins Utilized for Genome Engineering. Mol Biotechnol 65, 311-325 (2023). doi.org/10.1007/s12033-022-00567-0.

In some embodiments, a small molecule inhibitor can be to reduce and/or inhibit the activity or expression of RalA. For example, an RalA small molecule inhibitor can be 6-Amino-1,3-dimethyl-4-(4-(trifluoromethyl)phenyl)-1,4-dihydropyrano[2,3-c]pyrazole-5-carbonitrile, SCH-53239, SCH-54292, BQU57, RBC6, RBC8, and/or RBC10. In some embodiments, the RalA inhibitors provided herein reduce the RalA mRNA and/or polypeptide levels by about 10% to about 90% (e.g., about 10% to about 70%, about 10% to about 50%, about 10% to about 30%, about 10% to about 20%, about 20% to about 90%, about 20% to about 70%, about 20% to about 50%, about 20% to about 30%, about 30% to about 90%, about 30% to about 70%, about 30% to about 50%, about 50% to about 90%, about 50% to about 70%, about 70% to about 90%) of a control or reference level.

### Methods of Treatment

Provided herein are methods of treating obesity or a condition associated with obesity in a subject. Also provided herein methods of treating obesity or reducing the risk of obesity or a condition associated with obesity in a subject. In some embodiments, the methods include administering a therapeutically effective amount of a pharmaceutical composition comprising an RalA inhibitor.

In some embodiments, the subject has an increased risk of developing obesity and/or an obesity associated condition. For example, the subject may have a family history or a personal history of genetic disorders. In some embodiments, a subject is at increased risk of developing obesity if the subject has a high expression of RalA compared to a healthy individual. In some embodiments, the subject is a mammal. For example, the mammal can be a human, a monkey, a dog, a cat, a pig, a horse, a cow, a sheep, a goat, a rabbit, a mouse, or a rat.

Methods of treating obesity in a subject can include identifying a subject as having obesity and/or a condition associated with obesity. In some embodiments, conditions associated with obesity include type 2 diabetes, hyperinsulinemia, hepatic steatosis, weight gain, glucose intolerance, heart disease, chronic kidney disease, high cholesterol, gall bladder disease, high blood pressure, sleep apnea, gastroesophageal reflex disease, metabolic syndrome, acute pancreatitis, dyslipidemia, and cancer. In some embodiments, methods described herein includes identifying a subject as having hyperinsulinemia, type 2 diabetes, a BMI of 30 or over, a waist-hip ratio of greater than 1.0, prediabetes, and/or fatty liver disease. Examples of methods for identifying the subject as having obesity and/or a condition associated with obesity include, without limitation, physical examination (e.g., using body mass index (BMI) or waist-hip ratio (WHR)), and/or laboratory tests (e.g., blood or urine).

In some embodiments, a subject is considered overweight or obese by assessment of the subject's BMI, which is calculated by dividing a subject's weight in kilograms by the subject's height in meters squared. An adult having a BMI in the range of ˜18.5 to ˜24.9 kg/m² may be considered to have a normal weight; an adult having a BMI between ˜25 and ˜29.9 kg/m² may be considered overweight (pre-obese); and an adult having a BMI of ˜30 kg/m² or higher may be considered obese. In some embodiments, a subject is determined to have a WHR of 1.0 or greater prior to administering a RalA inhibitor. In some embodiments, a subject identified as having a WHR more than 0.9 in men or a WHR more than 0.8 in women.

Treating obesity or an obesity related condition according to the methods described herein include administering a therapeutically effective amount of an RalA inhibitor. A “therapeutically effective amount” is an amount sufficient to effect beneficial or desired results. For example, an effective amount is one that achieves a desired therapeutic effect, e.g., an amount necessary to treat a disease, or to reduce risk of development of disease or disease symptoms (also referred to as a prophylactically effective amount). An effective amount can be administered in one or more administrations, applications, or dosages. A therapeutically effective amount of a therapeutic compound (i.e., an effective dosage) depends on the therapeutic compounds selected. For example, a therapeutically effective amount of a pharmaceutical composition as provided herein can be effective to reduce body weight and fat mass (e.g., iWAT weight); improve insulin sensitivity; improve glucose tolerance and liver function; reduce hepatic glucose produce; reduce triglyceride content; increase energy expenditure and mitochondrial oxidative activity. In some embodiments, a therapeutically effective amount of a pharmaceutical composition as provided herein can be effective to improve at least one symptom of obesity or a condition associated with obesity.

The pharmaceutical composition (e.g., a pharmaceutical composition comprising an RalA inhibitor) provided herein can be administered one or more times per year (e.g., one time per year, two times per year, three times per year, four times per year, or five times per year) to one or more times per month (e.g., one time per month, two times per month, three times per month, four times per month, five times per month), including once every other month, once every three months, or twice a month. In some embodiments, the pharmaceutical composition provided herein can be administered one or more times per week (e.g., one time per week, two times per week, three times per week, four times per week, five times per week, six times per week, seven times per week, or more than seven times per week). The skilled artisan will appreciate that certain factors may influence the dosage and timing required to effectively treat a subject, including but not limited to the severity of the disease or disorder, previous treatments, the general health and/or age of the subject, and other diseases present. Moreover, treatment of a subject with a therapeutically effective amount of the therapeutic compounds described herein can include a single treatment or a series of treatments. Various factors can influence the actual amount used for a particular application. For example, the frequency of administration, duration of treatment, combination of other agents, site of administration, stage of disease (if present), and the anatomical configuration of the treated area may require an increase or decrease in the actual amount administered.

An effective duration for administering a pharmaceutical composition provided herein (e.g., a pharmaceutical composition comprising an RalA inhibitor) can be any duration that reduces the symptoms related to obesity and/or a condition associated with obesity, inhibits the activity or expression of RalA in a subject without producing significant toxicity to the subject. In some cases, the effective duration can vary from several days to several weeks, to several months, or longer. Multiple factors can influence the actual effective duration used for a particular treatment. For example, an effective duration can vary with the effective amount, frequency of administration, use of route of administration, and severity of the subject's condition.

In some embodiments, the methods and materials described herein can be effective to reduce expression of RalA in a subject having obesity and/or a condition associated to obesity by, for example 10, 20, 30, 40, 50, 60, 70, 80 or more percent. In some cases, the methods and materials described herein can be effective to reduce the severity of obesity and/or a condition associated with obesity in a subject.

In some cases, methods provided herein can be used to reduce the body weight of a subject. For example, a mammal in need thereof (e.g., a human having obesity and/or a condition associated with obesity) can be administered one or more (e.g., one, two, three, four, or more) RalA inhibitors to reduce the body weight of the subject. In some cases, the methods and materials provided herein can be used as described herein to reduce the body weight of a subject by, for example, 5, 10, 15, 20, 25, 30, or 35 or more percent.

In some cases, methods and materials provided herein can be used to reduce the fat mass (e.g., iWAT, BAT, and/or eWAT) in a subject. For example, a mammal in need thereof (e.g., a human having obesity and/or a condition associated with obesity) can be administered one or more RalA inhibitors to reduce the fat mass in the subject. In some cases, the methods and materials provided by, for example, 10, 20, 30, 40, 50, 60, 70, 80 or more percent.

In some cases, methods and materials provided herein can be used to improve insulin sensitivity in a subject. For example, a mammal in need thereof (e.g., a human having obesity and/or a condition associated with obesity) can be administered one or more RalA inhibitors to improve insulin sensitivity in the subject. In some cases, the methods and materials provided herein can be used as described herein to improve insulin sensitivity in a subject by, for example, 5, 10, 15, 20, 25, 30, or 35 or more percent.

In some cases, methods and materials provided herein can be used to reduce the blood glucose level in a subject. For example, a mammal in need thereof (e.g., a human having obesity and/or a condition associated with obesity) can be administered one or more RalA inhibitors to reduce the blood glucose level in the subject. In some cases, the methods and materials provided herein can be used as described herein to reduce the blood glucose level in a subject by, for example, 5, 10, 15, 20, 25, 30, or 35 or more percent.

In some cases, methods and materials provided herein can be used to reduce the liver weight in a subject. For example, a mammal in need thereof (e.g., a human having obesity and/or a condition associated with obesity) can be administered one or more RalA inhibitors to reduce the liver weight in the subject. In some cases, the methods and materials provided herein can be used as described herein to reduce the liver weight in a subject by, for example, 10, 20, 30, 40, 50, 60, 70, 80 or more percent.

In some embodiments, one or more RalA inhibitors described herein can be used as the sole active agent to treat a subject having obesity and/or a condition associated with obesity as described herein. In such embodiments, the pharmaceutical composition comprising one or more RalA inhibitors is not administered with a second pharmaceutical composition, wherein the second pharmaceutical composition is administered for the purpose of treating obesity or obesity-associated conditions.

In some cases, one or more RalA inhibitors described herein can be administered to a subject having obesity and/or conditions associated with obesity together with a second pharmaceutical composition used to treat (e.g., reduce the extent or severity of) obesity and/or conditions associated with obesity as described herein. For example, the second pharmaceutical composition that can be used to treat obesity and/or conditions associated with obesity, without limitation, naltrexone-bupropion, phentermine-topiramate, orlistat, diethylpropion, setmelanotide, phendimetrazine, benzphetamine, tirzepatide, a glucagon-like peptide-1 receptor (GLP-1) agonist, a glucose-dependent insulinotropic polypeptide (GIP), a GIP antagonist, an amylin agonist, a leptin agonist, a glucagon agonist, and/or any combinations thereof. In some cases, the GLP-1 agonist includes, without limitation, dulaglutide, exenatide, liraglutide, lixisenatide, semaglutide, and/or combinations thereof. In some cases, the GLP-1 agonist and/or GIP agonist comprises tirzepatide. In some cases, the amylin agonist comprises pramlintide. In some cases, the leptin agonist comprises Metreleptin. In some cases, the glucagon agonist comprises dasiglucagon, Baqsimi, or Gvoke.

In some embodiments, methods and materials provided herein can be used to improve the efficacy of the second pharmaceutical composition in a subject having obesity and/or conditions associated with obesity described herein. A mammal in need thereof (e.g., a human having obesity and/or conditions associated with obesity) can be administered one or more RalA inhibitors to improve the efficacy of the second pharmaceutical composition in a subject in need thereof. For example, methods and materials provided herein can be used to improve the efficacy of the second pharmaceutical composition by, for example, 10, 20, 30, 40, 50, 60, 70, 80, 90, 95, or more percent.

In some embodiments, methods and materials provided herein can be used to reduce the severity of the adverse effects of the second pharmaceutical composition in a subject having obesity and/or conditions associated with obesity described herein. In some embodiments, the adverse effects of the second pharmaceutical composition are muscle loss, nausea, and/or one or more psychiatric effects. A subject in need thereof can be administered one or more RalA inhibitors to reduce the severity of the adverse effects of the second pharmaceutical composition in a subject in need thereof. For example, methods and materials provided herein can be used to reduce the severity of the adverse effects of the second pharmaceutical composition by, for example, 10, 20, 30, 40, 50, 60, 70, 80, 90, 95, or more percent.

In some cases, one or more RalA inhibitors described herein can be administered to a subject having obesity and/or conditions associated with concurrent with the second pharmaceutical composition described herein. For example, methods and materials provided herein for treating or reducing the risk of obesity and/or conditions associated with obesity can include administering an RalA inhibitor to a subject together with a second pharmaceutical composition.

In some cases, one or more RalA inhibitors described herein are used in combination with the second composition used to treat obesity and/or conditions associated with obesity in a subject in need thereof. In some embodiments, the second composition can be administered to a subject in need thereof having obesity and/or conditions associated with obesity at the same time (e.g., administered as a single composition) or independently from the first composition. For example, RalA inhibitors described herein can be administered first, and the second pharmaceutical composition administered second, or vice versa.

In cases where one or more RalA inhibitors described herein are used in combination with a second pharmaceutical composition to treat obesity and/or conditions associated with obesity in a subject in need thereof, the second pharmaceutical composition can be administered at the same time or independently of the administration of RalA inhibitors as described herein. For example, one or more RalA inhibitors described herein can be administered before, during, or after the second pharmaceutical composition is administered.

Dosage, toxicity and therapeutic efficacy of the pharmaceutical composition (e.g., a pharmaceutical composition comprising an RalA inhibitor) disclosed herein can be determined by standard pharmaceutical procedures in cell cultures or experimental animals, e.g., ED50 (the dose therapeutically effective in 50% of the population). Compositions that exhibit high therapeutic indices are preferred. While compositions that exhibit toxic side effects may be used, care should be taken to minimize and reduce side effects. The data obtained from cell culture assays and animal studies can be used in formulating a range of dosage for use in humans. The dosage of such compounds lies preferably within a range of circulating concentrations that include the ED50 with little or no toxicity. The dosage may vary within this range depending upon the dosage form employed and the route of administration utilized. For any compositions used in the methods described herein, the therapeutically effective dose can be estimated initially from cell culture assays or animal models.

### Pharmaceutical Compositions

The RalA inhibitors described herein to treat or reduce the risk of obesity and/or conditions associated with obesity can be formulated into a composition (e.g., a pharmaceutical composition) for administration to a subject. For example, a therapeutically effective amount of RalA inhibitors described herein can be formulated together with one or more pharmaceutically acceptable carriers (additives) and/or diluents. A pharmaceutical composition can be formulated for administration in solid or liquid form including, without limitation, sterile solutions, suspensions, sustained-release formulations, tablets, capsules, pills, powders, and granules.

As used herein the language “pharmaceutically acceptable carrier” includes saline, solvents, dispersion media, coatings, antibacterial and antifungal agents, isotonic and absorption delaying agents, sterile aqueous or non-aqueous solutions, suspensions, emulsions, and the like, compatible with pharmaceutical administration. Examples of non-aqueous solvents include, without limitation, propylene glycol, polyethylene glycol, vegetable oils, and organic esters. Aqueous carriers include, without limitation, water, alcohol, saline, and buffered solutions. Acceptable carriers also can include physiologically acceptable aqueous vehicles (e.g., physiological saline) or other known carriers for oral administration. Pharmaceutical compositions are typically formulated to be compatible with its intended route of administration. Examples of routes of administration include oral, parenteral (including subcutaneous, intramuscular, intravenous, and intradermal), or inhaled administration. In some embodiments, administering comprises oral administration, injection, subcutaneous injection, intravenous injection, intramuscular injection, intraperitoneal injection, inhalation, or any combinations thereof.

In some embodiments, one or more RalA inhibitors described herein when formulated as a pharmaceutical composition can be formulated in an ingestible form or a topical form. For example, the pharmaceutical composition can be in the form of a liquid, solution, suspension, tablet, powder, granule, pill, capsule, gel, cream, mist, atomized vapor, aerosol, soft gelatin capsule, or hard gelatin capsule. For oral administration, tablets or capsules can be prepared by conventional means with pharmaceutically acceptable excipients such as binding agents, fillers, lubricants, disintegrants, or wetting agents. The tablets can be coated by methods known in the art. Liquid preparations for oral administration can take the form of, for example, solutions, syrups, or suspension, or they can be presented as a dry product for constitution with saline or other suitable liquid vehicle before use. Liquid preparations also can contain pharmaceutically acceptable additives such as suspending agents, emulsifying agents, non-aqueous vehicles, preservatives, buffer salts, flavoring agents, coloring agents, and sweetening agents as appropriate. Preparations for oral administration can be suitably formulated to give controlled release of the compound. In some embodiments, the pharmaceutical composition provided herein is formulated for oral ingestion, injection (e.g., intravenous, intradermal, subcutaneous, intramuscular, or intraperitoneal injection) or tissue-specific targeting.

The pharmaceutical composition provided herein can be administered by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories.

### EXAMPLES

The invention is further described in the following examples, which do not limit the scope of the invention described in the claims.

### Example 1: Methods and Materials

### Animals

RalA-floxed (Rala^{f/f}) mice were bred with adiponectin-promoter-driven Cre or Ucp1-promoter-driven Cre transgenic mice to generate fat depot-specific RalA KO (Rala^{AKO} or Rala^{BKO}) mice. All mice were on a C57BL/6J background, and all experiments were performed using littermates. Male mice were used for in vivo experiments and female mice were used only for primary preadipocyte isolation. Mice were fed with standard chow-diet (CD) (Teklad, 7912) or high-fat-diet (HFD) consisting of 60% calories from fat (Research Diets, D12492) for 8-12 weeks, starting from 8 weeks of age. Mice were housed in a specific-pathogen-free facility with a 12-hour light-dark cycle and given free access to food and water, except for the fasting period. The facility temperature and humidity were constantly kept at 22° C. and 50%, respectively.

### Cell Culture

### Primary Preadipocytes:

Inguinal white adipose tissue (WAT) from 2-3 8-week-old female mice was dissected, minced and digested in 5 mL 1 mg/mL collagenase (Sigma) for 15 minutes in a 37° C. water bath with gentle agitation.

DMEM/F12 medium (15 mM HEPES) with 10% FBS (growth medium) was added to stop digestion, and cells were filtered through 100-μm and 70-μm strainers. After centrifugation at 750 g, cells were plated onto a dish with growth medium. Once cells reached 90% confluence, preadipocytes were seeded into 12-well plates or imaging dishes for differentiation. Differentiation was induced in growth medium containing 0.5 mM IBMX, 5 μM dexamethasone, 1 μM rosiglitazone and 5 μg/mL insulin for 3 days. Medium was then switched to growth medium with rosiglitazone (day 3-5) and insulin (day 3-7). Day 7 onwards, cells were maintained in growth medium until they were 100% differentiated.

### Immortalized Adipocytes:

Primary preadipocytes from Rala^{f/f} mice were immortalized by retroviral transduction of pBabe-zeo-LT-ST (SV40) and selection by Zeocin⁷⁵. Single-cell clones were selected and tested for differentiation capacity. All clones used in this study displayed 100% adipocyte morphology after differentiation. To generate Rala KO cells, immortalized Rala^{f/f} (WT) preadipocytes were transduced with lentiviral Cre with 8 μg/mL Polybrene for 12 hours, then cultured in DMEM/F12-FBS medium. Cre recombinase efficiency was tested in preadipocytes and adipocytes. Once 95-100% confluency was reached (day 0), differentiation was induced as described above. On the day of the experiment, cells were starved in DMEM/F12 medium for 3 hours before treatments.

### Human Primary Preadipocytes (SGBS):

Cells were cultured in DMEM/F12-FBS medium supplemented with 3.3 mM biotin (Sigma, B4639) and 1.7 mM pantothenate (Sigma, P5155) and differentiated⁷⁶.

### 3T3-L1 Adipocytes:

Preadipocytes were cultured in high-glucose DMEM with 10% newborn calf serum (culture medium). At day 2 after confluency, differentiation was induced in culture medium containing 0.5 mM IBMX, 5 μM dexamethasone and 2 μg/mL insulin for 3 days. The medium was then switched to growth medium with insulin (day 3-7) or without insulin (day 7 to fully differentiated).

### Lenti-X 293T Cells:

Lenti-X 293T cells were cultured in high-glucose DMEM-FBS medium for packing lentivirus. When cells reached 100% confluency on a 0.01% poly-lysine-coated dish, third-generation lentiviral packaging plasmids (pLVX vectors, pMDLg/pRRE (Addgene #12251), pRSV-Rev (Addgene #12253) and pMD2.G (Addgene #12259)) were transfected into cells using lipofectamine 3000 (Life Technology) following the manufacturer's protocol. Fresh DMEM-FBS medium with 25 mM HEPES was added 12-16 hours after transfection. The lentivirus-containing medium was collected twice at 48 hour and 72 hour after transfection. After collection, the medium was spun at 300 g for 5 minutes to remove dead cells, then incubated with Lenti-X concentrator (Takara) at a 3:1 ratio at 4° C. overnight. The viral pellets were collected by centrifugation at 1,500 g for 45 minutes at 4° C. and reconstituted in DMEM/F12-FBS medium containing 8 μg/mL polybrene. Lentivirus was added to cells immediately after reconstitution.

### Reconstitution of RalA “T and RalA^{G23V} in RalA KO Preadipocytes

Immortalized RalA KO preadipocytes were transduced with concentrated Flag-RalA^{WT} or Flag-RalA^{G23V} lentiviral supernatants containing 8 μg/mL polybrene. At 24 hours after infection, the medium was changed to fresh DMEM/F12-FBS and expanded for differentiation. The expression of Flag-tagged protein was examined in fully differentiated cells by western blot.

### Gene Analysis in Clinical Cohorts

The transcriptomics data from abdominal subcutaneous WAT of 30 individuals with obesity and 26 healthy women were generated as previously described⁷⁷. Transcriptome profiles were obtained using GeneChip Human Gene 1.0 ST Arrays. Data were deposited in the National Center for Biotechnology Information (NCBI) Gene Expression Omnibus (GEO) under accession code GSE25402. Transcriptome profiles in the verification cohort were obtained from subcutaneous fat biopsies from 770 men participating in the (Metabolic Syndrome in Men) METSIM study⁷⁸. Transcriptomics and clinical data were retrieved from GEO (GSE70353). Obesity was defined as a BMI>30 kg m-2 in these analyses.

### Primary Mature Adipocyte Isolation

Minced WAT was digested in DMEM with 1 mg/mL collagenase (Sigma) for 25 minutes at 37° C. with gentle agitation. The cell suspension was filtered through a 100 μm cell strainer and centrifuged at 50 g for 3 minutes to separate floating mature adipocytes. Floating mature adipocytes were transferred to PBS with broad open tips and washed twice. Then, 1 mL mature adipocytes were lysed in 4 mL TRIzol (Life Technology) for RNA isolation.

### RNA Sequencing Analysis

RNA extractions from primary mature inguinal and epididymal adipocytes were performed using TRIzol (Life technologies) and PureLink RNA mini kit (Life Technologies), according to the manufacturer's instructions. RNA quality was checked by an Agilent TapeStation. Biological triplicates of isolated 500 ng RNA were used to prepare sequencing libraries using the TruSeq RNA Sample Preparation kit v.2 (Illumina), according to the manufacturer's protocol. Libraries were validated using a 2100 BioAnalyzer (Agilent), then normalized and pooled for sequencing using bar-coded multiplexing at a 90-bp single-end read length on an Illumina HiSeq 4000. Samples were sequenced to a median depth of 14 million reads.

### Bioinformatics Analysis

For RNA-seq, sequencing fastq files were generated automatically using the Illumina bcl2fastq2 Conversion Software. Read alignment and junction mapping to genome mm39 (GRCm39) and the mouse Genecode M30 annotation were performed using STAR (v.2.7.2b). Known splice junctions from mm 10 were supplied to the aligner and de novo junction discovery was also permitted. Differential gene expression analysis and statistical testing were performed using DESeq2 with an adjusted P value <0.05 as a cutoff. Raw gene counts were normalized to fragments per million mapped fragments (FPM) using DEseq2. FPM counts were filtered, centered by z score before gene clustering and heat map generation using GENE-E (v.3.0.215) or GraphPad Prism (v.8.4.3). For microarray data, gene matrix files were collapsed using the Collapse Dataset tool in Gene set enrichment analysis (GSEA) (v.4.3.2) using chip platform (GPL 13667) with collapsing mode (Mean_of_probes). The statistical significance of differential gene expression was assessed by ComparativeMarkerSelection module (v.11) from GenePattern (available on the world wide web at cloud.genepattern.org/gp/pages/index.jsf).

### Gene Expression Analysis

Tissue RNA was isolated with TRIzol reagent in combination with column (PureLink RNA mini, Invitrogen) according to the manufacturer's protocol. Complementary DNA was generated from 1 μg RNA using the cDNA Maxima Reverse Transcription kit (Thermo Fisher Scientific). The expression of mRNA was assessed by real-time PCR using the QuantStudio real-time PCR system and SYBR Green PCR master mix (Invitrogen). Gene expression was normalized to Cyclophilin A in murine tissues. Relative mRNA expression levels were calculated using averaged 2^{-ΔΔCt} values for each biological replicate. Primers are listed in Table 2.

TABLE 2
List of primers

| Gene Symbol | Forward |
|---|---|
| G6pc | CGACTCGCTATCTCCAAGTGA |
| Pepck | CCACAGCTGCTGCAGAACA |
| Fasn | GCTGTAGCACACATCCTAGGCA |
| Scd1 | ATCGCCTCTGGAGCCACAC |
| Acsl1 | TCCTACAAAGAGGTGGCAGAACT |
| Cpt1a | TGAGTGGCGTCCTCTTTGG |
| Cpt1b | CCAAACGTCACTGCCTAAGCT |
| Cpt2 | CAACTCGTATACCCAAACCCAGTC |
| Acadl | TCTTTTCCTCGGAGCATGACA |
| Adgre1 | CCCCAGTGTCCTTACAGAGTG |
| Col1a1 | GCTCCTCTTAGGGGCCACT |
| Col3a1 | CTGTAACATGGAAACTGGGGAAA |
| Rala | ATGGCTGCAAACAAGCCCA |
| Ucp1 | ACTGCCACACCTCCAGTCATT |
| Cidea | TGACATTCATGGGATTGCAGAC |
| Prdm16 | CAGCACGGTGAAGCCATTC |
| Ppargc0a | CCACTTCAATCCACCCAGAAA |
| Cox5b | GCGAAGTAACCTTGAAGCCA |
| Cox8a | CTTCGAGTGGACCTGAGC |
| Ndufs7 | CTTCTGTTCACGCTTGATCTTC |
| Atp5d | AAGATGCCAAAGGCTCCAG |
| Cytb | CCTTCATGTCGGACGAGGCTT |
| Nd2 | GCCTGGAATTCAGCCTACTAGC |
| Nd4 | CGCCTACTCCTCAGTTAGCCA |
| Cox1 | TAGCCCATGCAGGAGCATCA |
| Cox2 | ACCTGGTGAACTACGACTGCT |
| Cox3 | CTTCACCATCCTCCAAGCTTCA |
| Atp6 | TGGCATTAGCAGTCCGGCTT |
| Atp8 | TTCCCACTGGCACCTTCACC |
| Cyclophilin A | GAGCTGTTTGCAGACAAAGTTC |

### Protein Isolation and Western Blotting

Tissues or cells were lysed or homogenized in RIPA buffer with freshly added Halt Protease and Phosphatase Inhibitor Cocktail (Thermo Fisher). Lysates were rotated in a cold room for 30 minutes, then briefly sonicated and centrifuged at 17,000 g for 15 minutes at 4° C. Cleared supernatants were collected and concentrations were determined with a BCA protein assay kit (Pierce) and iTecan plate reader for quantification. Proteins were resolved by Tris-Glycine gel (Novex, Invitrogen) electrophoresis and transferred to nitrocellulose membranes. Individual 10 proteins were detected with the specific antibodies (OXPHOS ab110413, β-tubulin 2146S, phospho-Drp1 (Ser637) 4867S, phospho-HSL (Ser660) 45804S, HSL 4107S, MYC 2276S, Drp1 8570S, phosphor-AMPK (Thr172) 2535S, AMPK 5831S, RalA BD610221, β-actin 66009-1-Ig, Flag 66008-4-Ig, GFP 66002-1-Ig, and Sec512751-1-AP). Proteins were visualized on blots using fluorescent secondary antibodies with a Li-Cor system or on a film using HRP-conjugated secondary antibodies (Fisher Scientific) with SuperSignal West Pico Chemiluminescent substrate (Thermo Fisher). All primary antibodies were used at 1:1,000 dilution, fluorescent secondary antibodies were used at 1:5,000 dilution, and HRP-conjugated secondary antibodies were used at 1:8,000 dilution. Bands were quantified with ImageStudio or ImageJ.

### Body-Mass Composition

Body-mass composition was assessed in non-anesthetized mice using EchoMRI.

### Glucose Tolerance Test

Mice were fasted for 6 hours, then intraperitoneally (i.p.) injected with d-[+]-glucose in PBS at a dose of 2 g/kg body weight (BW) for CD-fed mice or 1.2 g/kg BW for HFD-fed mice. Blood glucose levels were measured before injection and at 15, 30, 60, 90 and 120 minutes after injection using the Easy Touch glucose monitoring system.

### Insulin Tolerance Test

Mice were fasted for 4 hours, then i.p. injected with human insulin (Sigma) in saline at a dose of 0.35 U/kg BW for CD-fed mice or 0.6 U/kg BW for HFD-fed mice. Blood glucose levels were measured as described above.

### Pyruvate Tolerance Test

Mice were fasted for 16 hours, then i.p. injected with pyruvate in PBS at a dose of 1.5 g/kg BW for HFD-fed mice. Blood glucose levels were measured as described above.

### Blood Parameters

Whole blood was taken from the facial vein. Blood glucose was measured with a glucose meter (Easy Touch) from the tail vein. Plasma was collected after centrifugation at 1,200 g at 4° C. for 10 minutes. Plasma triglyceride (TG) and plasma free fatty acid (FFA) levels were measured with an Infinity Triglycerides kit (Thermo Fisher) and NEFA kit (WAKO), respectively. Plasma insulin levels were measured with the Mouse Ultrasensitive Insulin ELISA kit (Crystal Chem, 90080), and leptin levels were measured with a Mouse Leptin ELISA (Crystal Chem, 90030) kit. Plasma aminotransferase (AST) and alanine aminotransferase (ALT) activity was measured with the Aspartate Aminotransferase Activity kit (Biovision, K753) and Alanine Aminotransferase Activity kit (Biovision, K752), respectively.

### HOMA-IR Calculation

Homeostasis model assessment of insulin resistance (HOMA-IR) is an index of overall insulin sensitivity⁷⁹. Glucose and insulin levels from overnight-fasted mice were measured as described above. The values were used to calculate HOMA-IR with the formula: fasting insulin (μU/L)×fasting glucose (nmol/L)/22.5.

### Hepatic Lipid TG Measurement

Frozen liver tissue (50-100 mg) was homogenized in 1 mL PBS. Then, 800 μL lysates were added to 4 mL extraction buffer. After thoroughly rotating for 30 minutes at room temperature (RT), the lipid phase was separated from the aqueous phase by centrifuging at 1,800 g for 20 minutes. A 0.2-mL lipid fraction in the organic phase was collected and transferred to a 1.5-mL tube to dry under a nitrogen stream in the fume hood. Then, 0.2 mL 2% Triton X-100 solution was used to solubilize the lipids. TG levels were determined using the Infinity Triglycerides kit (Thermo Fisher). The lipid amount was normalized to the liver lysate protein amount.

### Histology

For H&E staining, liver tissue was collected and fixed in 10% formalin. For adipocyte size quantification, H&E slides were imaged using a Keyance brightfield microscope or a Nikon confocal microscope with Texas Red excitation and emission filters. Adipocyte size was assayed using Adiposoft in ImageJ and an in-house-developed pipeline with Cell Pro-filer. For Oil-Red-O staining, liver tissue was fixed in 4% Paraformaldehyde (PFA) at 4° C. for 24 hours, then transferred to 20% sucrose/PBS for 24 hours. Afterwards, tissue was embedded in O.C.T. (Sakura) with dry ice and ethanol. Frozen tissue blocks were sectioned and stained with Oil-Red-O.

### Indirect Calorimetric Measurements

For metabolic cage studies, mice were individually housed in Promethion metabolic cages maintained at 22° C. under a 12-hour light-dark cycle. Before the experiment, mice were adapted to the metabolic cages for 2 days. The monitoring system recorded and calculated food intake, locomotor activity, oxygen consumption, CO₂ production, respiratory exchange ratio (RER), and energy expenditure (EE). Mice were provided with free access to water and food during the entire measurement. The data were exported with ExpeData software (Sable Systems), and EE was analyzed using ANCOVA with BW as a covariate by a web-based Call tool⁸⁰.

### Respiration Measurement

### Intact Cells:

The cellular oxygen consumption rate (OCR) was measured using an eXF96 Extracellular Flux Analyzer and analyzed by Agilent Seahorse Wave Software (Seahorse Bioscience). Before performing the assay, 2,500 primary preadipocytes were seeded and differentiated in XF96 microplates. Once fully differentiated, the adipocyte culture medium was changed to assay medium containing 25 mM glucose, 1 mM pyruvate and 2 mM L-glutamine, and 0.5 mM carnitine without phenol red or sodium bicarbonate for 3 hours. Before taking measurements, cells were incubated in a CO₂-free incubator for 15 minutes. Basal rates of respiration were measured in assay medium and followed with sequential injections of oligomycin (2 μM), FCCP (0.5 μM) and rotenone with antimycin A (each 0.5 μM). Oxygen consumption values were normalized to total protein content.

### Isolated Mitochondria:

Mitochondria was isolated from HFD-fed mice. The OCR was performed with 2.5 μg of isolated mitochondria as described elsewhere³³.

### Fatty Acid Oxidation Assay

Fully differentiated primary adipocytes in 24-well plates were incubated in 0.5 mL DMEM per well containing 1 mM carnitine and 0.5 μCi per well and [¹⁴C]-PA for 60 minutes at 37° C. Afterwards, 360 μL medium was collected and added to 40 μL of 10% BSA in a 1.5-mL tube with a filter paper in the cap. Then, 200 μL of 1 M perchloric acid was added to the tube and the cap was immediately closed tightly and incubated at RT. After 1 hour, captured CO₂ and acid-soluble metabolites (ASMs) were used to measure radioactivity. The cells were lysed in NaOH/SDS buffer (0.3 N/0.1%) to measure protein concentration. Fatty acid oxidation (FAO) rates were normalized to protein content.

### Glucose Uptake Assay

### In Vivo:

CD-fed mice were fasted for 6 hours; and 10 μCi [³H]-deoxy-glucose or [¹⁴C]-deoxy-glucose was i.p. injected alone or spiked with 1.2 g/kg glucose into each mouse. Then, 30 minutes after injection, plasma and tissues were collected and snap frozen until further processing. The accumulation of deoxy-glucose-phosphate in different tissues was determined using as described elsewhere²⁶.

In vitro:

Fully differentiated primary adipocytes were fasted in serum-free medium for 3 hours before the assay. A glucose uptake-Glo assay was performed according to the manufacturer's protocol (Promega).

### Confocal Microscope Imaging

### Live Cells:

Fully differentiated adipocytes were cultured in a glass-bottom dish (Cellvis) and incubated in phenol-red-free DMEM (imaging medium) with 100 nM Tetramethylrhodamine, methyl ester (TMRM) (Thermo Fisher) for 30 minutes to indicate mitochondrial membrane potential. Boron-dipyrromethene (BODIPY) 493/503 (final 5 μg/mL, Life Technology) was added to label lipid droplets for the last 15 minutes. Cells were then washed three times with imaging medium. Live-cell images were obtained with a Nikon AIR confocal microscope with ×100 or ×60 oil immersion objectives. For time-lapse imaging, pictures were taken every 10 minutes.

### Fixed Cells:

Fully differentiated primary adipocytes were cultured in a glass-bottom chamber (Lab-Tek). On the day of the experiment, cells were serum-starved for 3 hours and treated with 100 nM insulin. After 15 minutes, the medium was removed, cells were fixed with ice-cold methanol and incubated at −20° C. for 10 minutes. Cells were then washed twice with PBS and blocked with 10% goat serum in PBS with 0.1% Triton X-100 at RT for 30 minutes. After blocking, cells were incubated with primary antibodies (1:50 dilution) at 4° C. overnight followed by incubation with secondary antibodies (1:2,000 dilution) for 1 hour at RT. Cells were washed three times with PBS before imaging with a Nikon AIR confocal microscope using a ×100 oil immersion objective.

### 4D Mitochondria Live-Cell Imaging and Analysis

A custom-built lattice light-sheet microscope designed by the Betzig

Laboratory HHMIJanelia/UCBerkeley was used to image fully differentiated adipocytes⁸¹. The 488-nm and 560-nm lasers were used to excite BODIPY and MitoTracker Red. A Multiple Bessel Beam Light Sheet Pattern with NA max 0.4, NA min 0.38 was used, which has a 75-μm sheet length. The measured lateral resolution was 330 nm, and the z resolution was 700 nm. To quantify mitochondrial motility and dynamics, cell segmentation, mitochondria segmentation and mitochondria tracking were performed. Single cells were first cropped using ImageJ and Python scripts for all 60 time points. MitoGraph was used to segment the mitochondria in each cell. Based on the segmented mitochondrial skeleton, MitoTNT was used to track mitochondria and perform motility calculations as described elsewhere⁸². Mitochondria displaying high motility were used for further fusion and fission dynamic analysis. Mitochondria fusion and fission levels were measured by the number of detected events per 1,000 mitochondrial skeleton nodes for each frame. Only the highly active events (counts >3) were used for comparison.

### Lipolysis

### In Vitro:

Fully differentiated primary adipocytes in a 24-well plate were serum-starved in lipolysis medium (2% BSA-phenol-red-free DMEM) for 3 hours. For insulin treatment, 100 nM insulin was added to cells for 30 minutes starting at 2.5 hours of starvation. After starvation, the medium was replaced with 0.5 mL fresh lipolysis medium with vehicle, 1 μM CL-316,243 (CL), 100 nM insulin or in combination. The medium was collected 1 hour after incubation at 37° C. Released FFAs and free glycerol levels were measured using 100 μL medium with a NEFA kit (WAKO) and Free Glycerol Reagent (Sigma) according to the manufacturer's protocol.

### In Vivo:

CD-fed mice were used for in vivo lipolysis. For CL-induced lipolysis, ad libitum-fed mice were i.p. injected with PBS or CL (1 mg/kg) for 60 minutes. Circulating FFAs and free glycerol levels were measured using 2 μL plasma with a NEFA kit (WAKO) and Free Glycerol Reagent (Sigma), respectively. For insulin-suppressed lipolysis, overnight-fasted mice were i.p. injected with insulin (0.5 U/kg) for 60 minutes. Circulating FFAs and free glycerol levels were measured at the indicated conditions.

### Electron Microscopy

### Adipose Tissue:

Dissected adipose tissue was immediately fixed with 2-3 drops of fixative buffer (2% paraformaldehyde and 2.5% glutaraldehyde in 0.15 M sodium cacodylate buffer, pH 7.4). Fat tissues were gently removed and fixed at RT. After 2 hours of incubation, tissues were further cut into around 1 mm3 cubes and immersed in fixative buffer overnight at 4° C. Tissue cubes were postfixed in 1% osmium 0.15 M sodium cacodylate (SC) buffer for 1-2 hours on ice, followed by five 10-minute washes in 0.15 M SC buffer, then rinsed in ddH₂O on ice. Washed tissues were stained with 2% uranyl acetate for 1-2 hours at 4° C. and then dehydrated in an ethanol series (50%, 70%, 90%, 100% and 100%, for 10 minutes each time), and dried in acetone for 15 minutes at RT. Dried tissues were infiltrated with 50: 50% acetone: Durcupan for 1 hour or longer at RT, followed by incubation in 100% Durcupan overnight. The next day, embedded tissues in Durcupan were placed in a 60° C. oven for 36 to 48 hours. Ultrathin sections (60 nm) were cut on a Leica microtome with a Diamond knife and then post-stained with both uranyl acetate and lead. Images were obtained using a Jeol 1400 plus TEM equipped with a Gatan digital camera.

### Immortalized Cells:

Fully differentiated cells in a 6-well plate were quickly fixed with 2% glutaraldehyde in 0.1 M SC buffer (pH 7.4) at RT for 15 minutes and then incubated at 4° C. for 15 minutes. Afterwards, cells were scraped down and pelleted by centrifugation. Cell pellets were post-fixed in 1% OsO₄ in 0.1 M SC buffer for 1 hour on ice. The cells were stained all at once with 2% uranyl acetate for 1 hour on ice, then dehydrated in a graded series of ethanol (50-100%) while remaining on ice. The cells were then subjected to one wash with 100% ethanol and two washes with acetone (10 minutes each) and embedded with Durcupan. Sections were cut at 60 nm on a Leica UCT ultramicrotome and picked up on 300 mesh copper grids. Sections were post-stained with 2% uranyl acetate for 5 minutes and Sato's lead stain for 1 minute. Images were obtained using a Jeol 1400 plus TEM equipped with a Gatan digital camera.

### CAMP Measurement

To induce cAMP production, fully differentiated primary adipocytes were stimulated with 1 μM CL for 5 minutes. Cells were then immediately lysed in lysis buffer (0.1 N HCL). The CAMP levels were measured with the Direct CAMP Enzyme Immunoassay kit (Sigma) according to the manufacturer's protocol.

### Pulldown and Co-Immunoprecipitation

### Active RalA Pulldown:

Fully differentiated primary adipocytes or immortalized adipocytes were serum-starved for 3 hours in DMEM and treated with 100 nM insulin, if needed, for the indicated time. After two washes with ice-cold TBS, cells were lysed in RalA buffer (25 mM Tris, 130 mM NaCl, 10 mM MgCl₂, 10% glycerol, 0.5% NP-40 and EDTA-free protease inhibitor) and lysates were incubated at 4° C. for 15 minutes and then cleared by centrifugation. Protein concentrations were measured with the DC protein assay (Bio-Rad) and 0.5-1 mg protein was incubated at 4° C. with 20 μL GST-^{Δ}RalBP1 agarose beads (Millipore) for 45 minutes or 20 μL Anti-Flag M2 Affinity gel (Sigma) overnight. After incubation, beads were washed three times with RalA buffer and boiled at 65° C. in 2×SDS buffer for 10 minutes.

### Pulldown:

HEK293T cells cultured in 15 cm dishes were transfected with Flag-RalA^{WT} or GFP-PP2Aa. At 48 hours after transfection, the cells were washed twice with ice-cold TBS, then lysed on ice with 1 mL lysis buffer (25 mM Tris-HCl, 130 mM NaCl, 10 mM MgCl₂, 10% glycerol, 0.5% NP-40 and EDTA-free protease inhibitor). Cell lysates were rotated for 15 minutes at 4° C. and centrifuged for 15 minutes at 17,000 g at 4° C. Flag-RalA^{WT} lysates were incubated with 20 μL Anti-Flag M2 Affinity gel (Sigma) at 4° C. After 2 hours of rotation, the empty M2 or Flag-RalA^{WT} beads were washed three times with lysis buffer, followed by incubation with GFP-PP2Aa lysates at 4° C. overnight. The next day, the beads were washed three times with washing buffer (25 mM Tris-HCl, 40 mM NaCl, 30 mM MgCl₂, 0.5% NP-40 and EDTA-free protease inhibitor) and boiled in 2×SDS buffer at 65° C. for 10 minutes. For GTPγS and GDP loading to Flag-RalA^{WT} beads, washed beads were rinsed with loading buffer (20 mM Tris, 50 mM NaCl, 1 mM mM EDTA) and incubated with 2 mM GTPγS or 200 μM GDP in loading buffer for 1 hour at 25° C. with 50 g agitation. After loading, 10 mM MgCl₂ was added to stop the loading, and the loaded beads were incubated with GFP-PP2Aa lysates as described above.

### Co-Immunoprecipitation:

Co-transfected cells at 70-80% confluency were washed twice with ice-cold TBS and lysed in 0.5 mL lysis buffer or Drp1 buffer (25 mM Tris, 50 mM NaCl, 0.5 mM MgCl₂, 10% glycerol, 0.5% NP-40 and EDTA-free protease inhibitor). Lysates were cleared by centrifugation, and protein concentrations were measured using the BCA assay (Pierce). Then, 0.5-1 mg protein was incubated with 20 μL Anti-Flag M2 Affinity gel (Sigma) at 4° C. After overnight gentle rotation, beads were washed three times with the washing buffer (the same as described above) or Drp1 wash buffer (25 mM Tris, 50 mM NaCl, 0.5 mM MgCl₂, 0.1% NP-40 and EDTA-free protease inhibitor) and boiled in 2×SDS buffer at 65° C. for 10 minutes.

### Vector Construction

pMIG-PP2Aa (#10884), pMIG-PP2Ab (#13804) and pcDNA3.1-Drp1 (#34706) plasmids were purchased from Addgene and subcloned into mEGFP-C1 (#54759) and pCMV-Myc-3B vectors. RalA^{WT}, RalA^{G23V} and RalA^{S28N} plasmids were subcloned into a pLVX vector with 3×Flag tag for lentiviral production.

### Statistics and Reproducibility

All in vivo animal experiments were randomized by genotype. The investigators were not blinded to allocation during experiments and outcome assessment. All in vitro cell experiments were not randomized. There was no predetermination of sample size; sample size was chosen based on available animal or cell numbers. Negative values or Prism-detected outliners were excluded from the analyses due to poor sample quality or samples lost during processing. Statistical analyses were performed using GraphPad Prism (v.8.4.3). All experiments were performed at least three times independently. Data distribution was assumed to be normal without formal testing. For comparison between two groups, datasets were analyzed by a two-tailed Student's t-test. For experiments with a two-factorial design, multiple comparisons were analyzed by two-way ANOVA to determine the statistical significance between groups based on one variable. Differences in EE were calculated with CalR using ANCOVA with BW as a covariate. The significance of the correlations between gene expression with BMI and HOMA values were calculated using Spearman's correlation test. P<. 05 was deemed significant.

### Example 2: White Adipocyte-Specific Rala Deletion Protected Mice from HFD-Induced Obesity

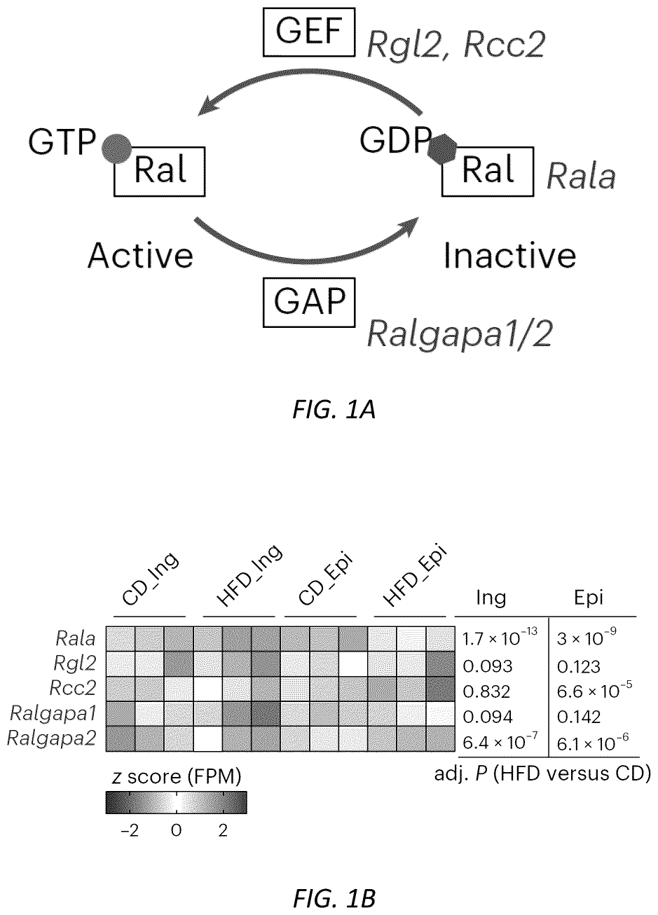

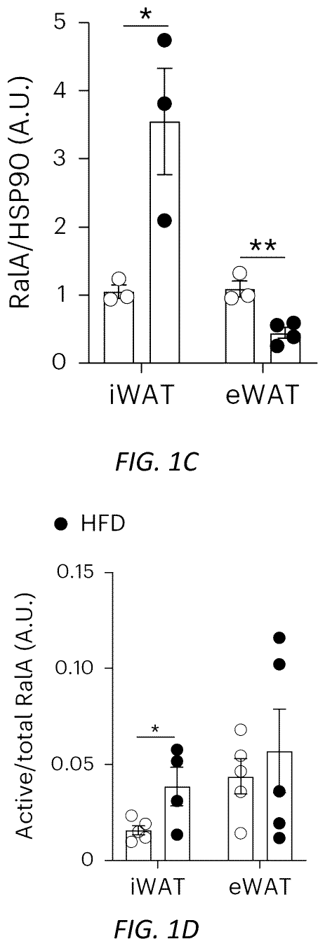

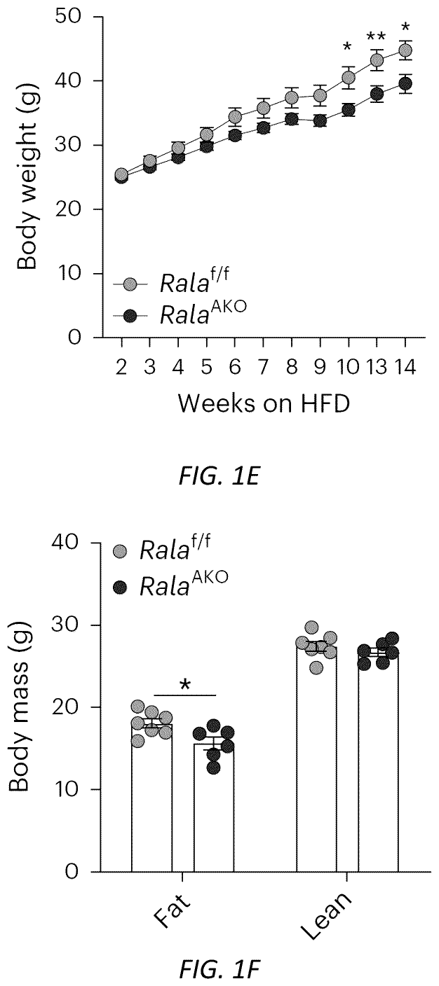

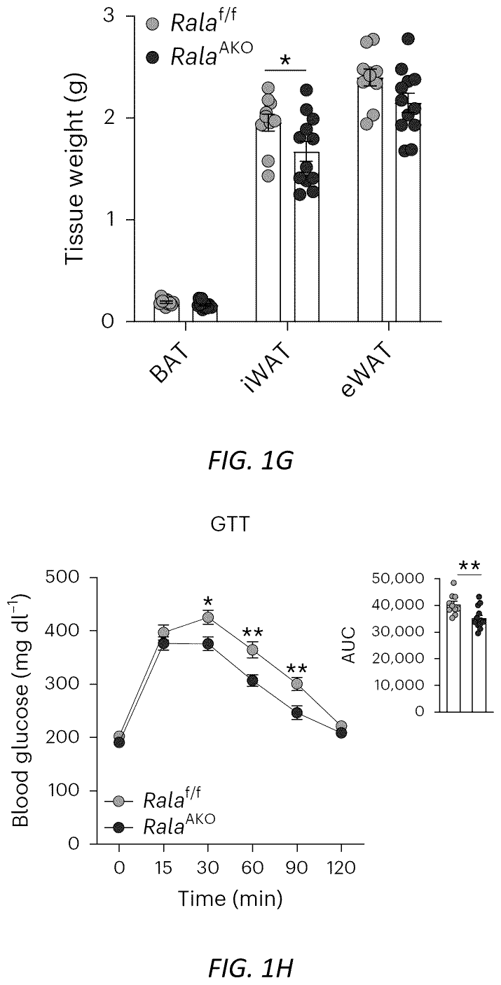

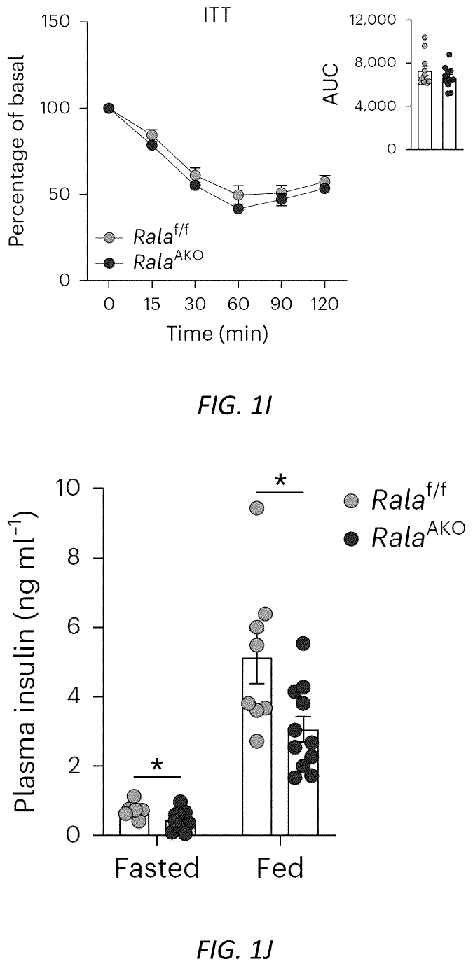

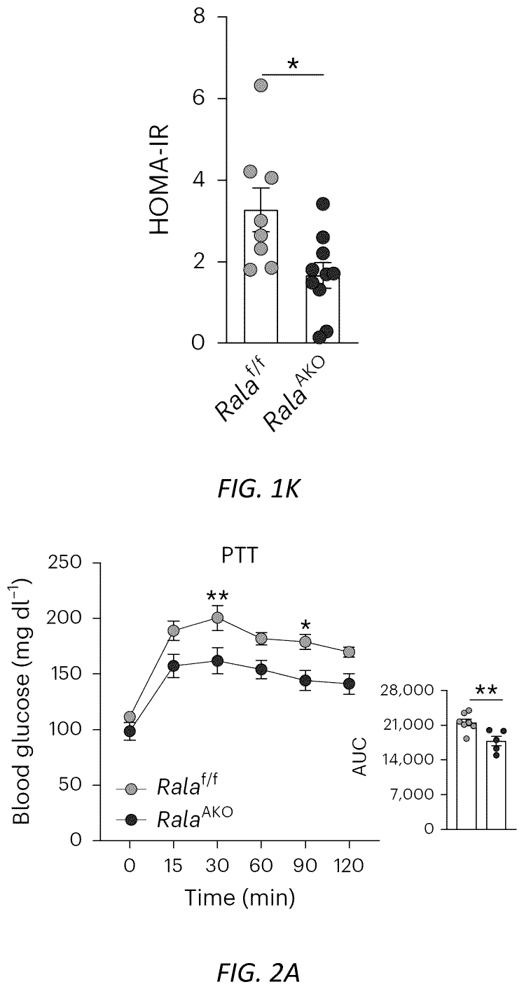

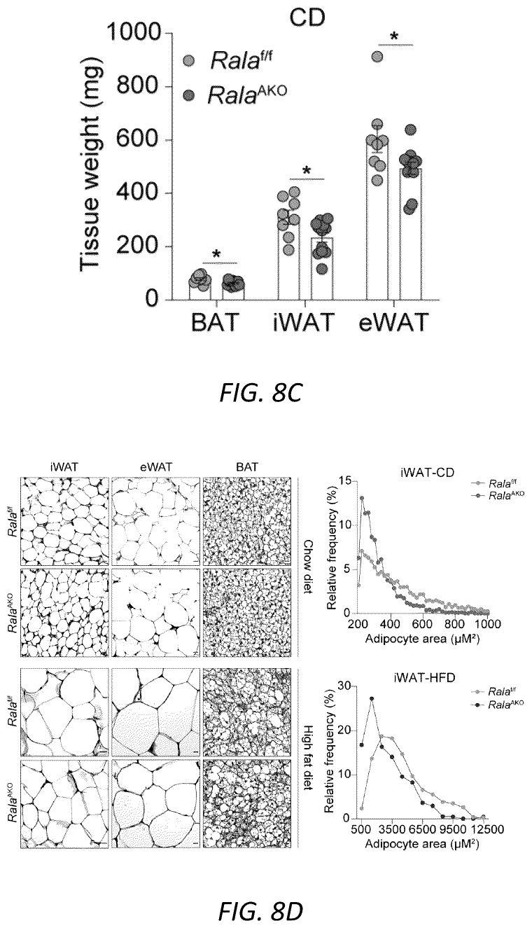

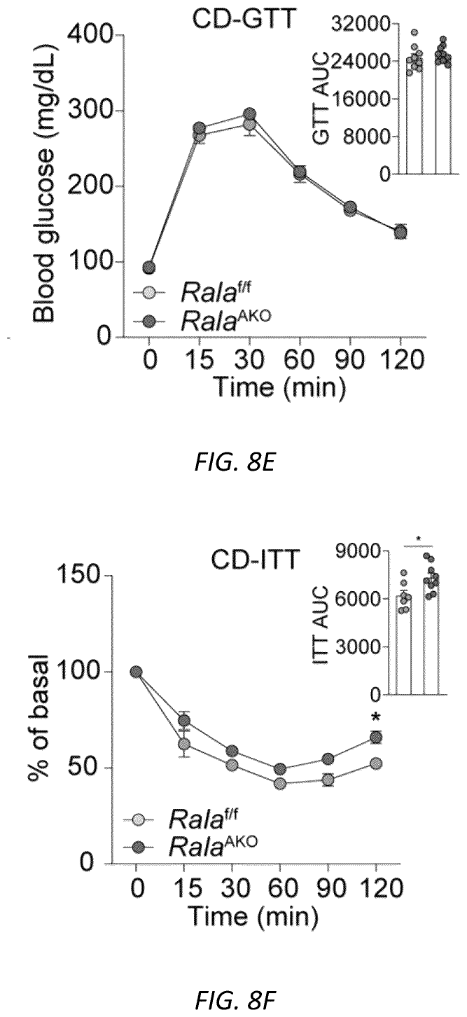

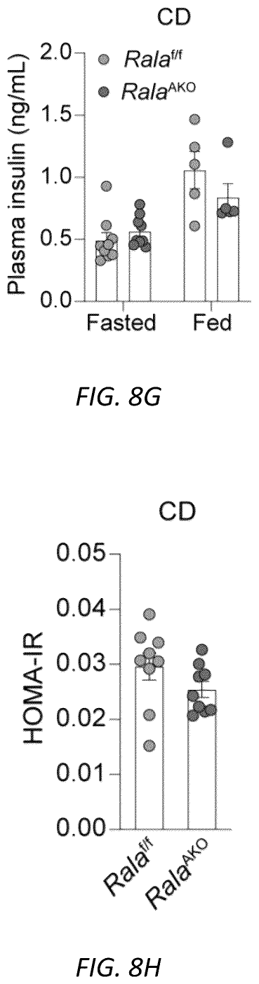

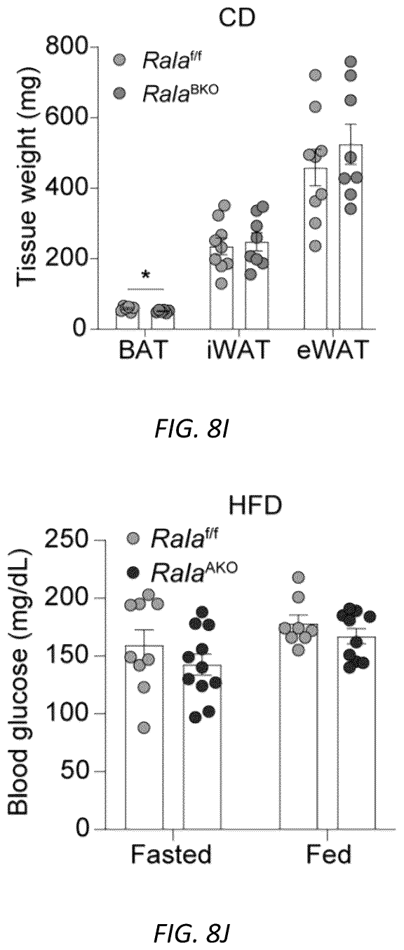

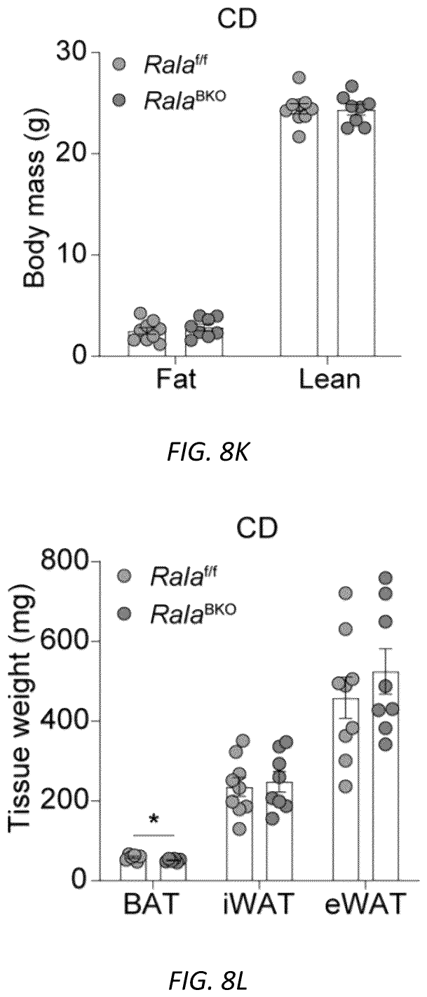

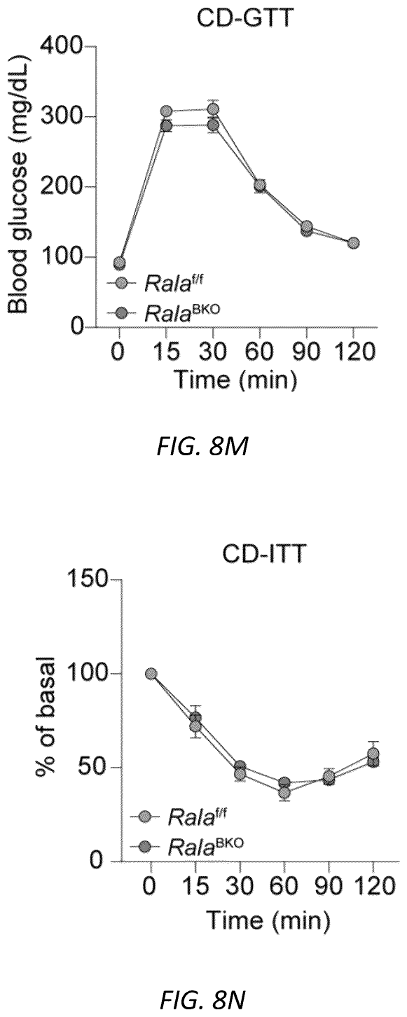

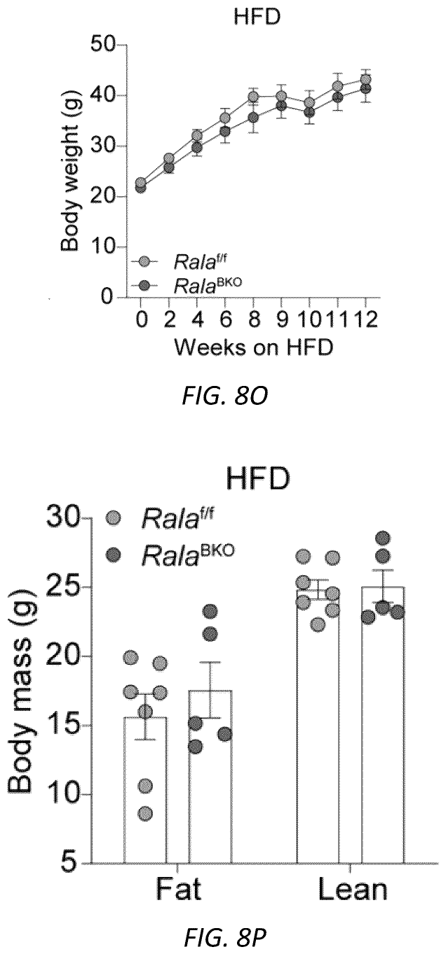

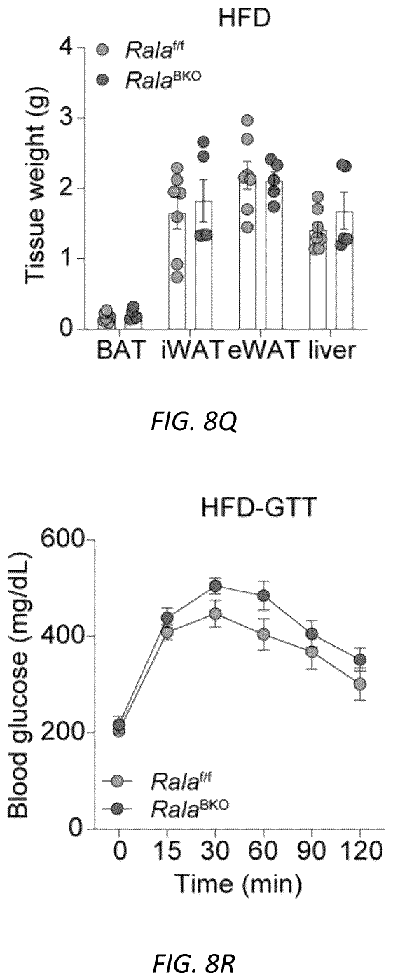

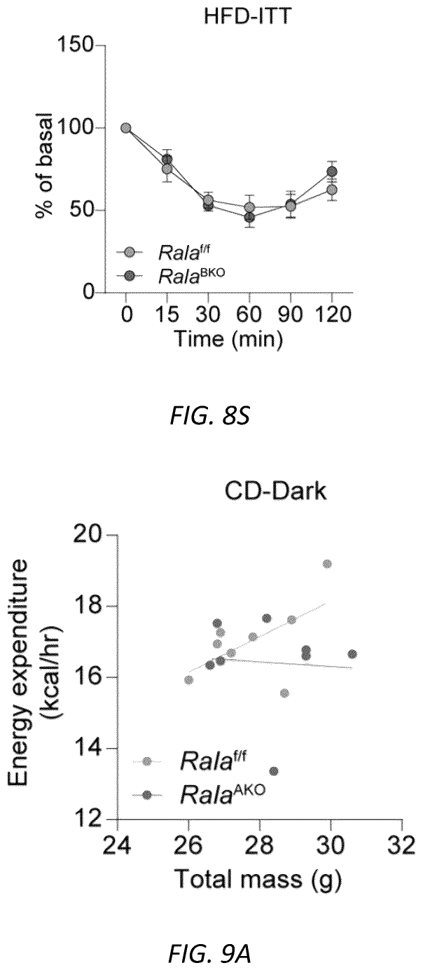

RNA sequencing (RNA-seq) analysis from the isolated mature adipocytes derived from control and HFD-fed mice²⁸ revealed that Rala expression was significantly upregulated in adipocytes from epididymal WAT (eWAT) and iWAT during obesity development, whereas Ralgapa2 expression was downregulated (FIGS. 1A and 1B). In addition, RalA protein content increased in mature adipocytes from iWAT of obese mice (FIGS. 1C and 8A), accompanied by the elevation of RalA-GTP binding (FIGS. 1D and 8B). These positive correlations seemed to be exclusive for WAT as no changes were detected in RalA levels in BAT after HFD feeding (FIG. 8C). Together, these findings showed that adipocyte RalA activity is constitutively elevated in obesity. To further explore the role of RalA in glucose homeostasis and energy metabolism, adipocyte-specific Rala knockout (Rala^{AKO}) mice were generated by crossing Rala-floxed mice with adiponectin-Cre transgenic mice. Compared to Rala^{f/f} littermates, Rala^{AKO} mice had a greater than 90% decrease in the level of RalA protein in primary adipocytes from WAT and BAT and an approximately 50% decrease in the whole WAT, without changes in the liver (FIG. 8D). Insulin-stimulated GTP binding of RalA was diminished in WAT of Rala^{AKO} mice compared to the control mice. Moreover, the primary adipocytes showed reduced RalA activity (FIG. 8E).

Depletion of RalA produced a reduction in insulin-stimulated glucose uptake in iWAT and BAT (FIGS. 8F-8H). Brown adipocyte-specific KO (RalA^{BKO}) mice were created by crossing RalA-floxed mice with UCP1-Cre transgenic mice (FIG. 8E). In contrast to what was observed in brown adipocyte-specific RalGAP KO mice, glucose uptake was reduced in the BAT of RalA^{BKO} mice (FIGS. 8J-8L). Notably, insulin-stimulated glucose uptake was mostly restricted to brown fat, and it was shown that RalA is dispensable for glucose uptake into eWAT in both gain-of-function and loss-of-function models. To further examine whether the impact of RalA on glucose uptake in adipocytes occurs in a cell-autonomous manner, primary white adipocytes were generated by inducing differentiation of iWAT stromal vascular cells from the control and the KO mice. Microscopy and subcellular fractionation showed that knocking out RalA completely prevented the translocation of GLUT4 from intracellular sites to the plasma membrane in response to insulin (FIGS. 8M-8N). Moreover, insulin-stimulated glucose uptake in the KO cells was significantly reduced without disturbing the upstream insulin signaling (FIGS. 80 and 8P).

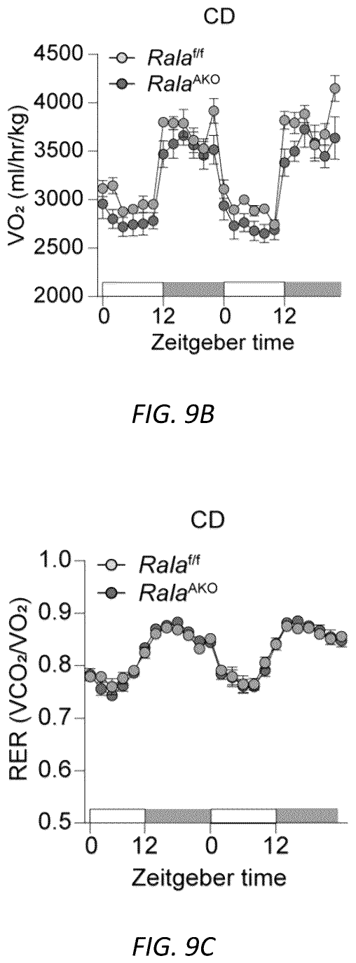

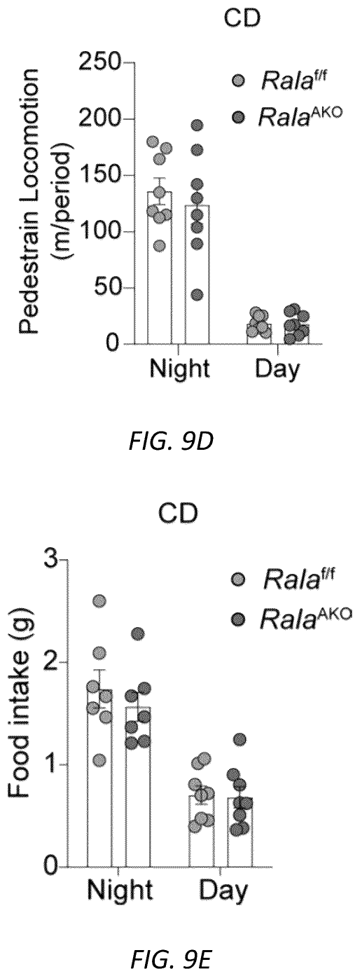

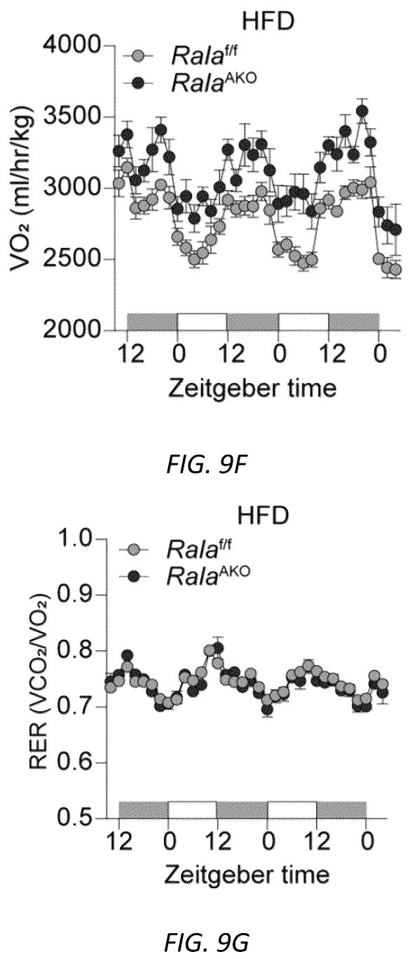

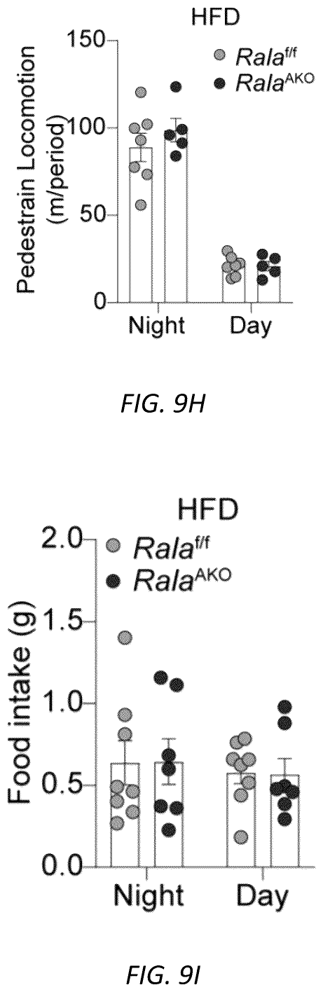

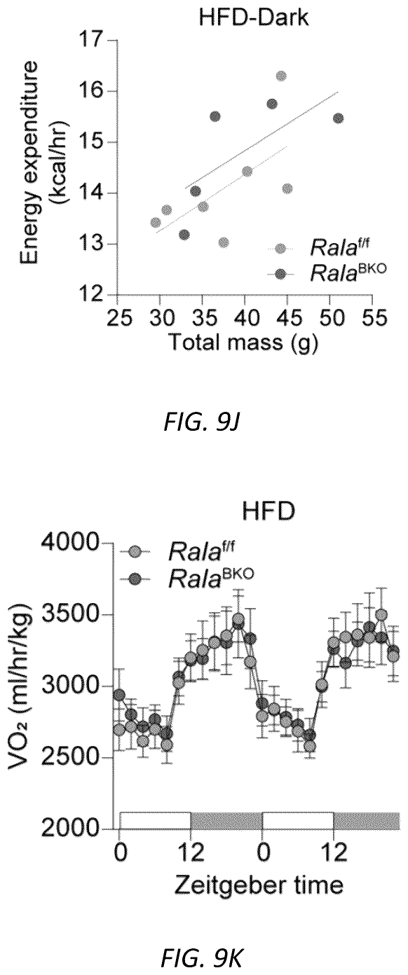

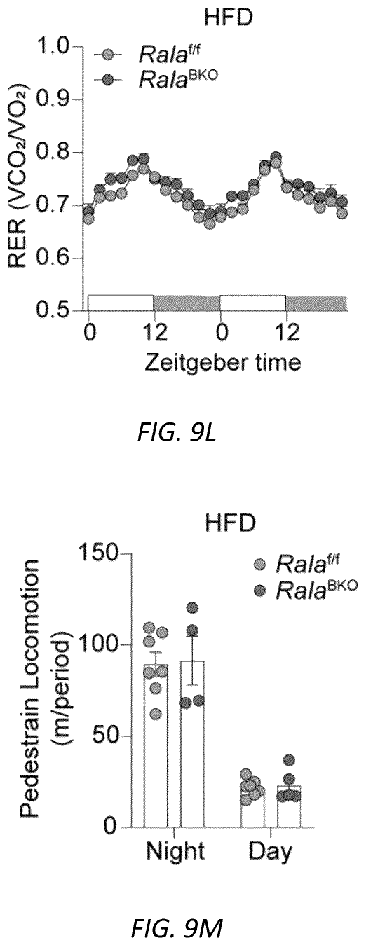

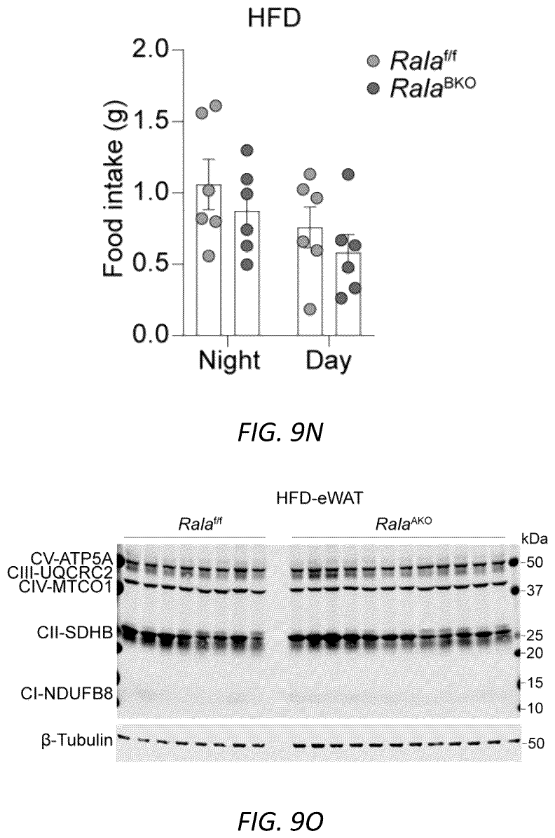

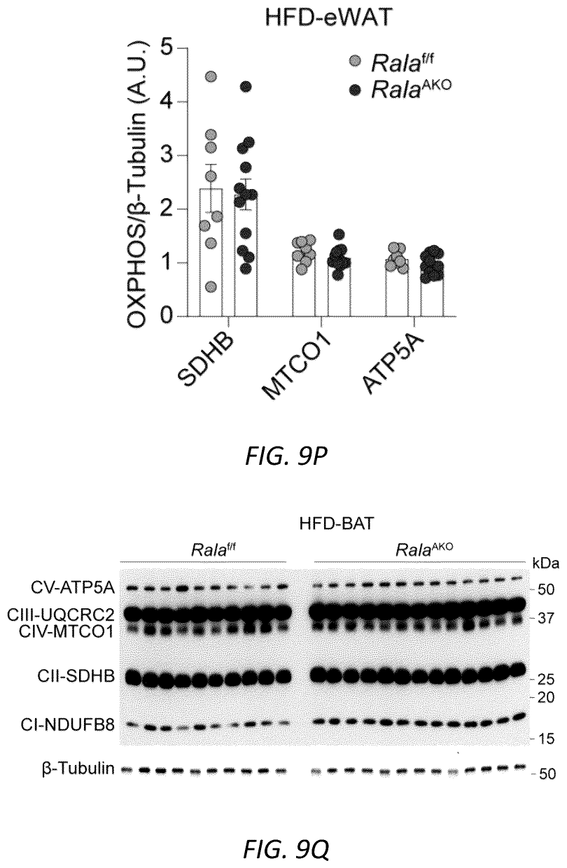

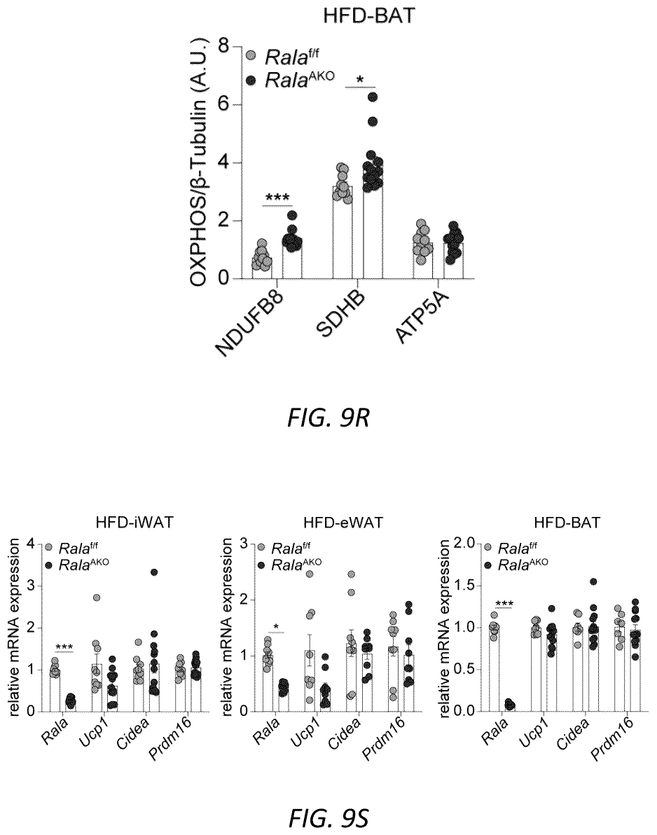

Adipocyte-specific deletion of Rala had no effect on body weight in CD-fed mice, although these mice displayed a reduction in fat mass and depot weight (FIGS. 9A-9C). Adipocytes from iWAT were considerably smaller than those found in eWAT from mice fed with CD²⁹. Rala^{AKO} mice had smaller adipocytes in iWAT compared to the control mice fed with CD, whereas adipocyte size was comparable in eWAT and BAT between the genotypes (FIG. 8D). While Rala^{AKO} mice on CD showed no difference in glucose tolerance, there was a slight reduction in insulin tolerance compared to Rala^{f/f} mice (FIGS. 8E and 8F). Insulin levels and homeostasis model assessment of insulin resistance (HOMA-IR) in Rala^{AKO} mice were not different from the control mice fed with CD (FIGS. 8G and 8H); however, Rala^{AKO} mice gained significantly less weight than the control littermates when challenged with 60% HFD (FIG. 1E), including a marked reduction of fat mass, with no change in lean body mass (FIG. 1F). Further analyses revealed that iWAT weight was reduced in Rala^{AKO} mice, with no difference observed in eWAT and BAT (FIG. 1G). HFD increased adipocyte size in all fat depots from wild-type (WT) mice, but the effect was most pronounced in iWAT. On the contrary, HFD-fed Rala^{AKO} mice displayed a trend toward smaller adipocytes in iWAT compared to the control mice, but not in eWAT or BAT (FIG. 8D). HFD-fed Rala^{AKO} mice exhibited a marked improvement in glucose tolerance compared to the control mice, with no change in insulin tolerance (FIGS. 1H and 1I), but with reduced insulin levels and improved HOMA-IR (FIGS. 1J and 1K). Fasting glucose levels were comparable between the genotypes fed with either HFD or CD (FIGS. 81 and 8J).

To investigate which adipose tissue depot is responsible for the reduced weight gain in Rala^{AKO} mice fed with HFD, Rala^{BKO} mice were placed on HFD. Although CD-fed Rala^{BKO} mice showed a reduction in BAT weight, presumably due to reduced glucose uptake, there were no differences in the overall fat mass or depot weight compared to the control mice (FIGS. 8K and 8L). Glucose and insulin tolerance tests results (GTTs and ITTs) were identical between the genotypes on the control diet (FIGS. 8M and 8N). Moreover, no differences in body weight, fat mass, tissue weight, GTT or ITT were observed in HFD-fed Rala^{BKO} mice (FIGS. 80-8S). HFD-fed mice exhibited insulin resistance in BAT. For example, RalA activation was already decreased in WT mice on HFD compared to the control diet. Thus, these results suggested that specific Rala deletion in WAT, especially in iWAT, protects mice against obesity.

### Example 3: Loss of RalA in WAT Ameliorated HFD-Induced Hepatic Steatosis

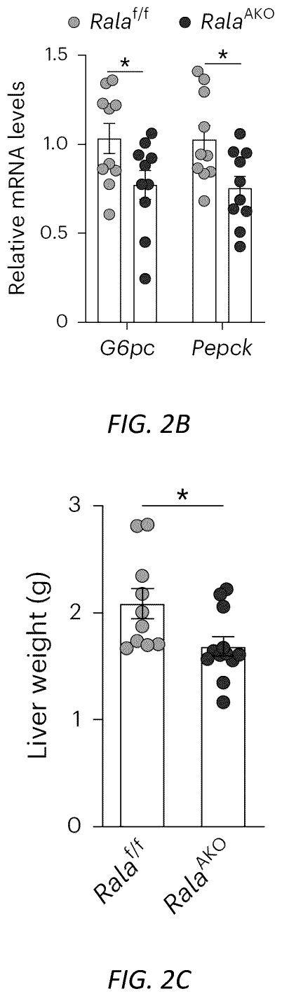

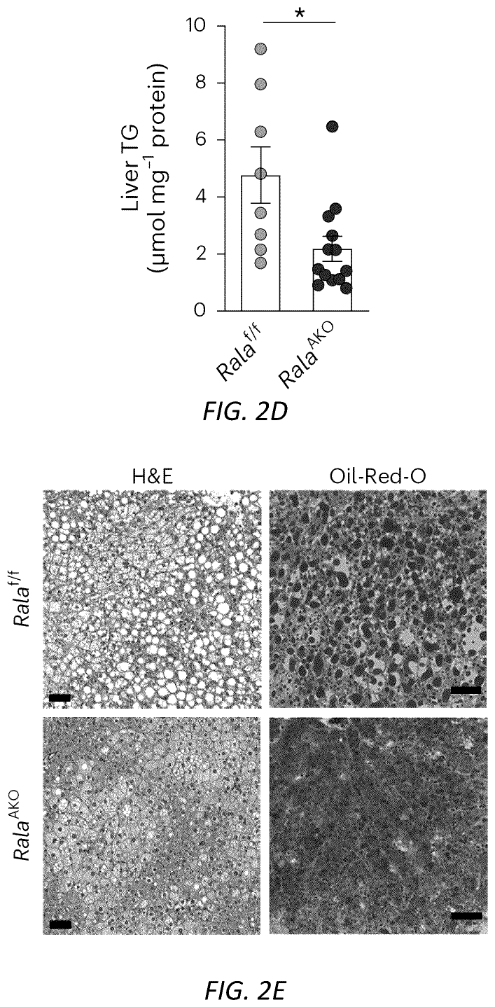

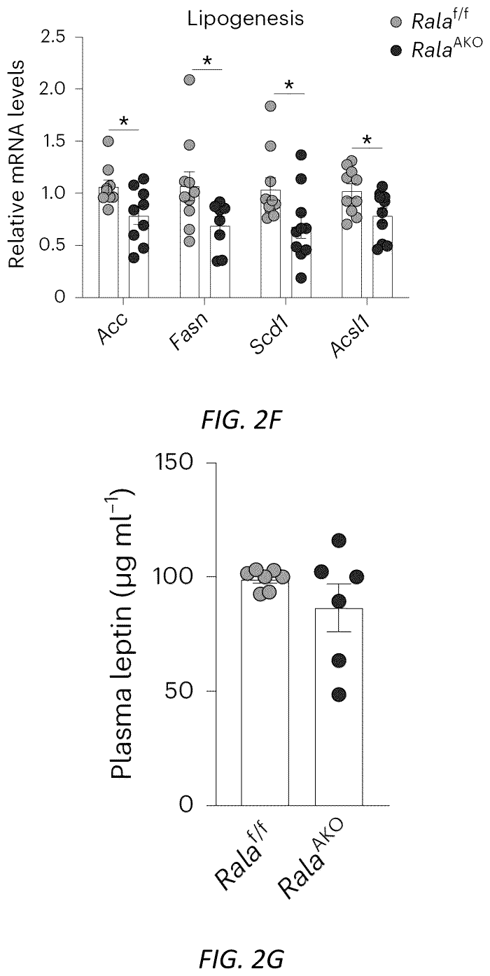

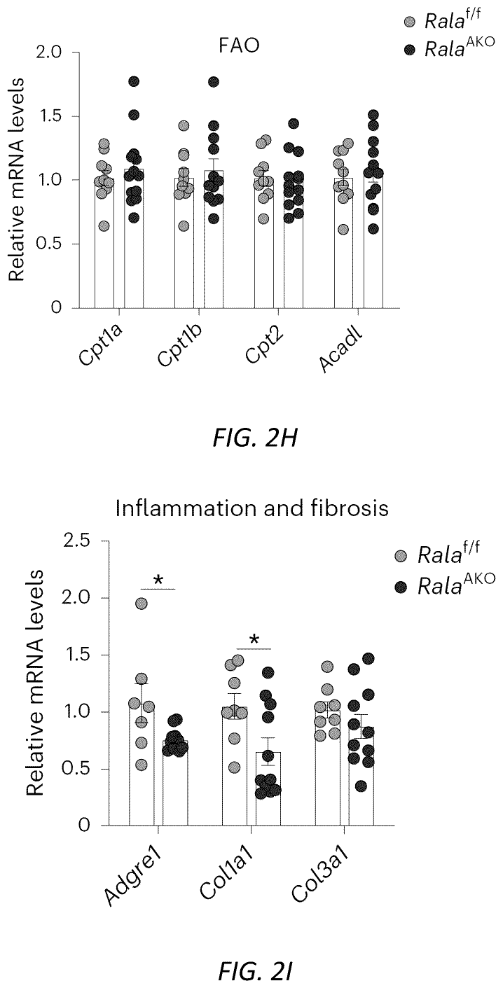

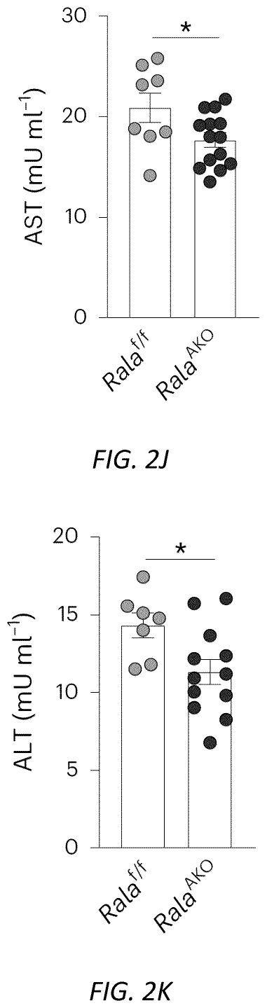

To test if the improved glucose handling was due to reduced hepatic glucose production, a pyruvate tolerance test (PTT) was performed in HFD-fed Rala^{f/f} and Rala^{AKO} mice. Rala^{AKO} mice exhibited substantially lower glucose excursions following pyruvate challenge compared to the control mice (FIG. 2A). There was a significant downregulation of the hepatic gluconeogenic genes G6pc and Pepck (FIG. 2B). These data demonstrated that adipocyte-specific Rala deletion improves glucose homeostasis partially through reduced hepatic glucose production.

Liver weights and triglyceride (TG) content were significantly reduced in HFD-fed Rala^{AKO} mice compared to the control mice (FIGS. 2C and 2D). Both hematoxylin and eosin (H&E) and Oil-Red-O staining demonstrated less lipid accumulation in the livers of Rala^{AKO} mice (FIG. 2E). Consistent with the histology results, lipogenic genes (Acc, Fasn, Scd1, and Acsl1) were expressed at lower levels in the livers of Rala^{AKO} mice (FIG. 2F); however, plasma leptin levels (FIG. 2G) and hepatic expression of genes related to fatty acid oxidation (FAO) (FIG. 2H) were unchanged in Rala^{AKO} mice. In addition, inflammatory (Adgre1) and fibrosis-related (Col1a1 and Col3a1) genes were expressed at lower levels in the livers of Rala^{AKO} mice (FIG. 2I), as were aspartate aminotransferase (AST) and alanine aminotransferase (ALT) activities (FIGS. 2J and 2K). No difference was observed in the liver weights in Rala^{BKO} mice compared to the control mice fed with HFD (FIG. 8Q). Together, these findings showed that WAT-specific deletion of Rala systemically regulates lipid metabolism to ameliorate liver steatosis and damage in obesity.

### Example 4: RalA Deficiency in WAT Increased Energy Expenditure and Mitochondrial Oxidative Phosphorylation

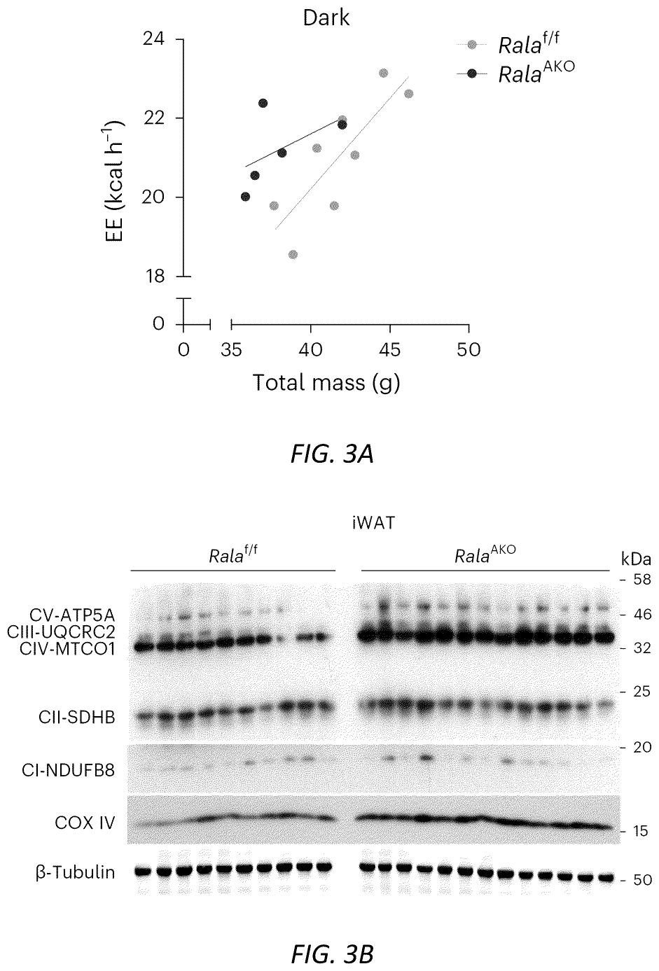

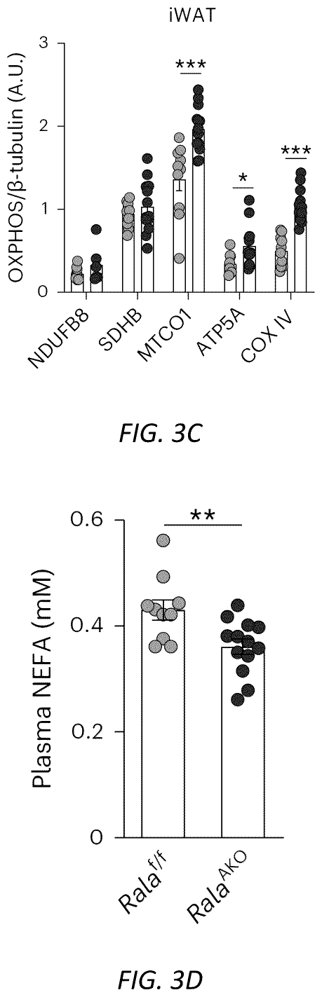

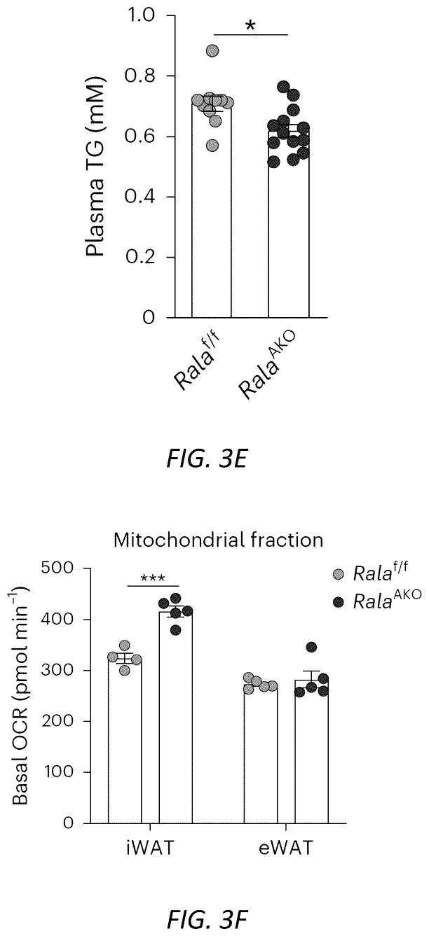

To investigate why Rala deletion in the adipose tissue protects the mice from HFD-induced hepatic steatosis, weight gain, and glucose intolerance, energy metabolism in Rala^{AKO} mice with metabolic cage studies were tested. While Rala ablation in adipocytes did not affect energy metabolism and food intake in mice fed with CD (FIGS. 9A-9E), HFD-fed Rala^{AKO} mice displayed an increase in energy expenditure during the dark phase as determined by analysis of covariance (ANCOVA) using body weight as a covariate (FIG. 3A). Concordantly, oxygen consumption in Rala^{AKO} mice was similarly increased compared to the control mice (FIG. 9F), although there was no difference in respiratory exchange rate (RER), locomotor activity, or food intake between the genotypes (FIGS. 9G-9I). In contrast, Rala^{BKO} mice fed with HFD were identical to the control littermates in energy expenditure, O₂ consumption, RER, locomotor activity, and food intake (FIGS. 9J-9N). These results demonstrated that Rala deficiency specifically in WAT increases energy expenditure.

Next, the expression of mitochondrial proteins in fat depots was tested. Oxidative phosphorylation (OXPHOS) proteins were markedly increased in iWAT of Rala^{AKO} mice (FIGS. 3B and 3C), but not in eWAT (FIGS. 9O and 9P). Complex I and complex II levels were modestly increased in BAT of Rala^{AKO} mice (FIGS. 9Q and 9R). As HFD-fed Rala^{BKO} mice did not show an improved metabolic phenotype, systemic metabolic improvement in Rala^{AKO} mice is likely rather than a cell-autonomous BAT function. In this regard, plasma free fatty acid (FFA) and TG levels in HFD-fed Rala^{AKO} mice were lower (FIGS. 3D and 3E). To test the possible involvement of a generalized browning of iWAT, thermogenic markers were examined. Ucp1, Cidea and Prdm16 expressions were identical between the genotypes in all three fat depots, indicating that the improvement in energy expenditure in Rala^{AKO} mice did not reflect the development of beige adipose tissue (FIG. 9S).

### Example 5: Rala Knockout in White Adipocytes Increased Mitochondrial Activity and Fatty Acid Oxidation

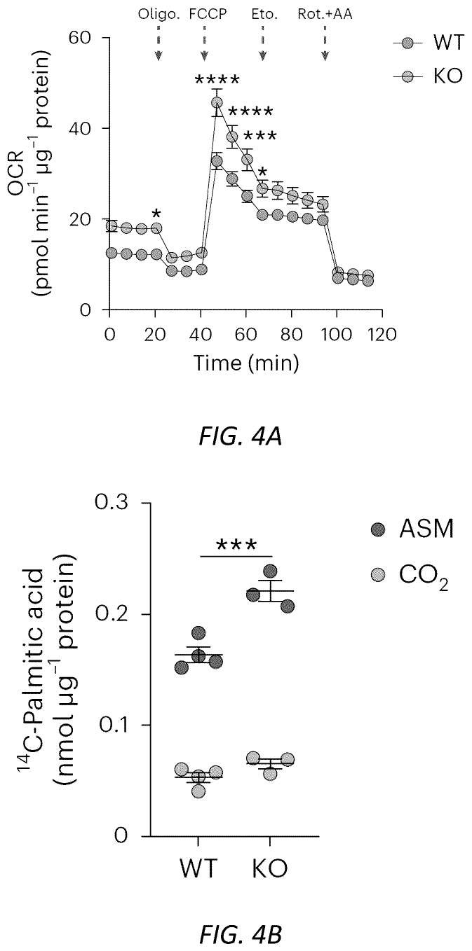

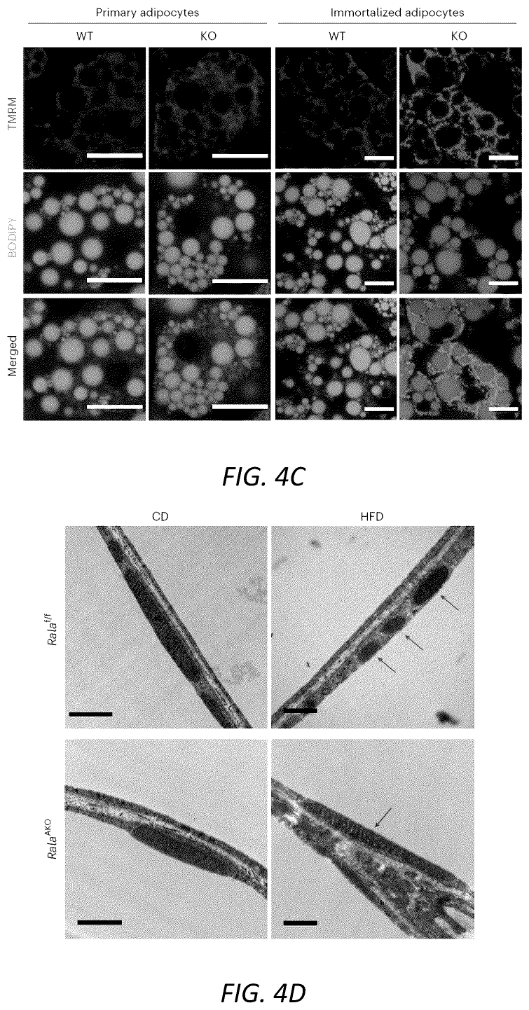

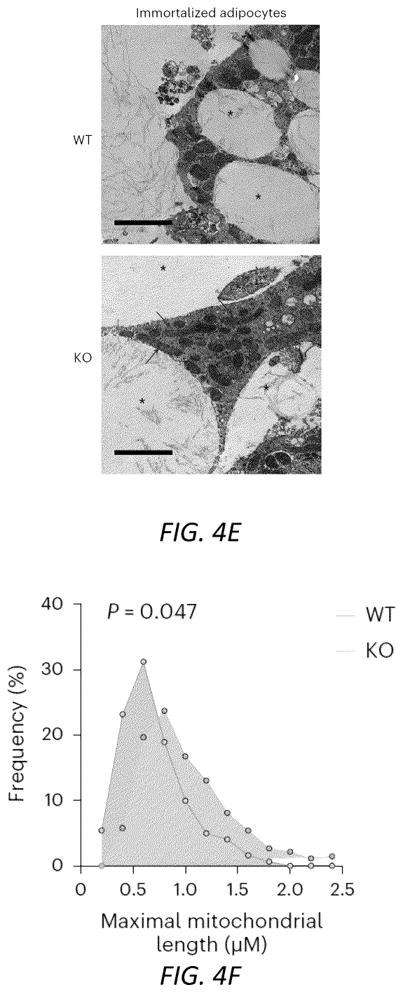

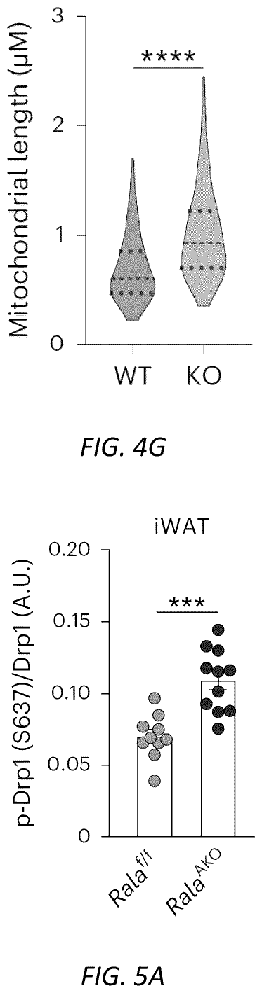

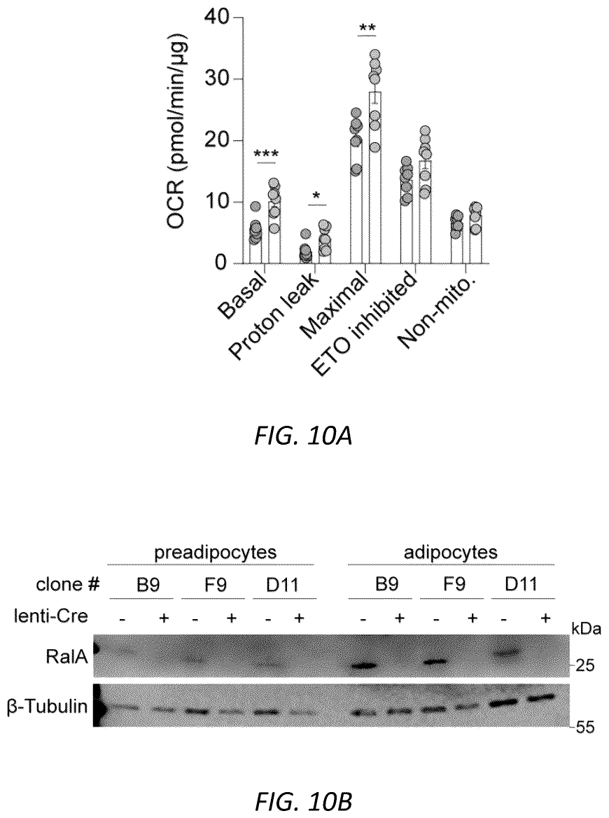

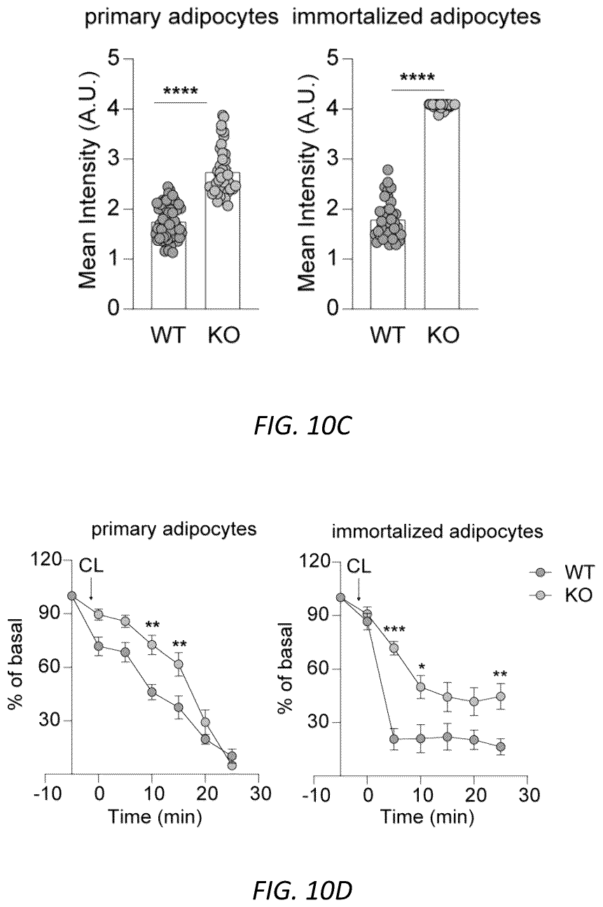

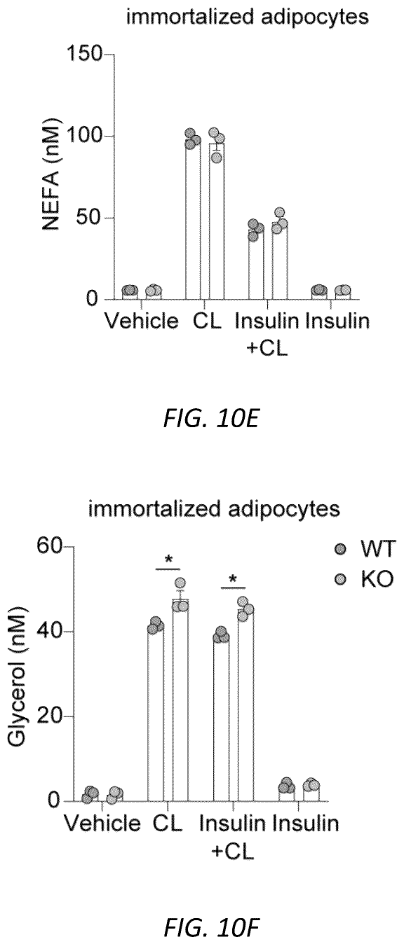

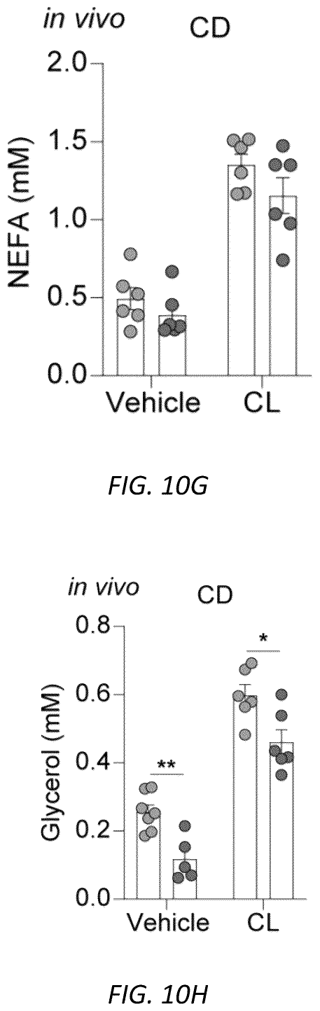

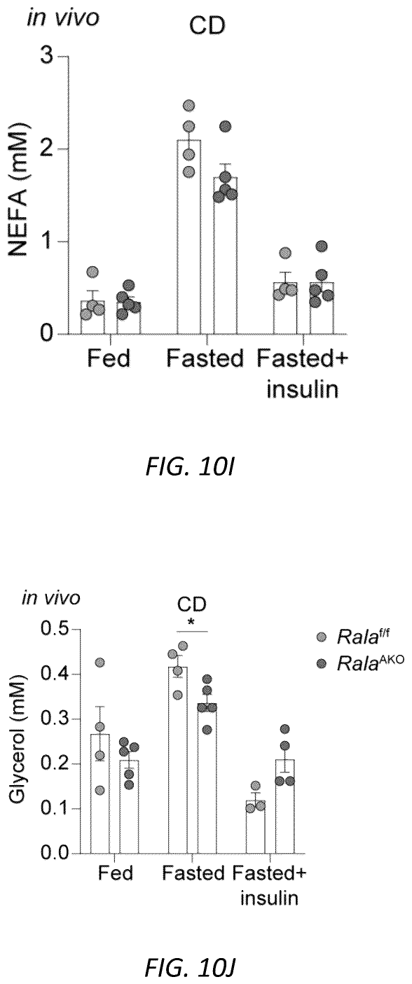

The mechanisms underlying the improved energy metabolism in Rala^{AKO} mice were investigated, and mitochondrial activity in adipocytes was directly assessed. Measurements of the basal respiration revealed that oxygen consumption rate (OCR) had increased in mitochondria isolated from KO iWAT compared to the control mice, but was similar in eWAT mitochondria of Rala^{f/f} and Rala^{AKO} mice (FIG. 3F). It was also noted that both the basal and maximal respiration were higher in primary differentiated adipocytes from the KO mice. The difference in maximal respiration was blunted by the addition of the CPT1 inhibitor etomoxir that can block FAO (FIGS. 4A and 10A). To investigate the role of RalA plays in controlling FAO, cells were incubated with (¹⁴C)-labeled palmitic acid (PA) and measured its oxidation to either acid-soluble metabolites (ASMs) or CO₂ in WT and KO white adipocytes. Consistent with the OCR results, FAO was higher in the KO adipocytes compared to the WT adipocytes (FIG. 4B). These data showed that RalA KO in WAT increases energy expenditure due to increased mitochondrial oxidation activity.

To ensure that these studies reflected the activity of RalA, an immortalized preadipocyte line was generated from Rala^{f/f} mice and induced Rala deletion by transducing cells with Cre lentivirus. The Cre recombinase completely ablated RalA in preadipocytes and the fully differentiated adipocytes (FIG. 10B). BODIPY staining demonstrated that both primary and immortalized preadipocytes from WT and KO mice were fully differentiated. As an orthogonal approach, live-cell imaging was performed using the cell permeant fluorescent dye, TMRM, to detect mitochondrial membrane potential (MtMP), which can reflect electron transport and OXPHOS in active mitochondria. KO adipocytes exhibited a higher TMRM signal intensity than their WT counterparts (FIGS. 4C and 10C). To specify the ability of TMRM to detect mitochondrial depolarization in active mitochondria, the β3-adrenergic receptor agonist CL-316,243 (CL) was used to induce mitochondrial membrane depolarization³². The TMRM signal declined quickly after administration of the agonist, demonstrating that TMRM stains only active mitochondria (FIG. 10D).

Lipolysis can drive mitochondrial oxidative metabolism in adipocytes³³. To rule out a possible role for lipolysis as the primary driver of increased oxidative capacity of Rala KO adipocytes, in vitro and in vivo lipolysis assays were performed. CL robustly stimulated FFA and glycerol release to the same extent in KO and WT immortalized adipocytes. The molar ratio of FFA to glycerol was approximately 3:1 (FIGS. 10E and 10F). Additionally, there were no differences in CL-induced FFA and free glycerol production in Rala^{f/f} and Rala^{AKO} mice (FIGS. 10G and 10H). It was also tested whether Rala^{AKO} mice are defective in the suppression of FFA release by insulin. Insulin suppressed CL-induced FFA release by approximately 50% in both WT and KO cells (FIG. 10E). A single injection of insulin reduced FFA levels in both control and Rala^{AKO} mice to the same extent (FIG. 10I). Notably, KO adipocytes displayed a mild increase in glycerol release in the presence of CL, whereas Rala^{AKO} mice showed a mild decrease of plasma glycerol levels either in the presence of CL or after fasting (FIGS. 10F, 10H, and 10J). Taken together, these results suggested that the absence of RalA in adipocytes enhances mitochondrial oxidative activity without affecting FFA supply.

### Example 6: Targeted Rala Knockout Protected Against Obesity-Induced Mitochondrial Fission in iWAT

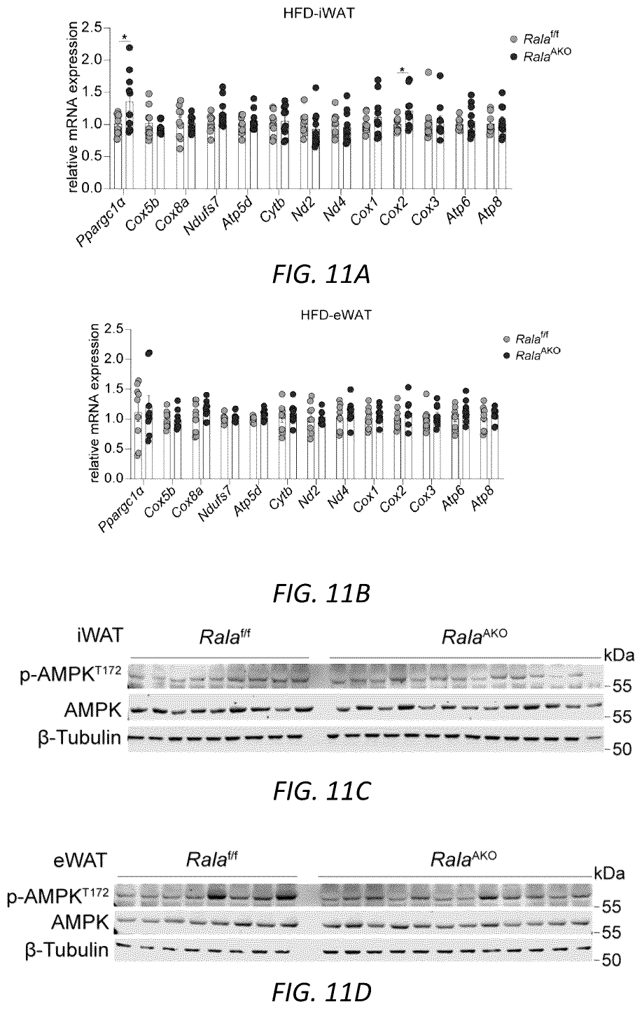

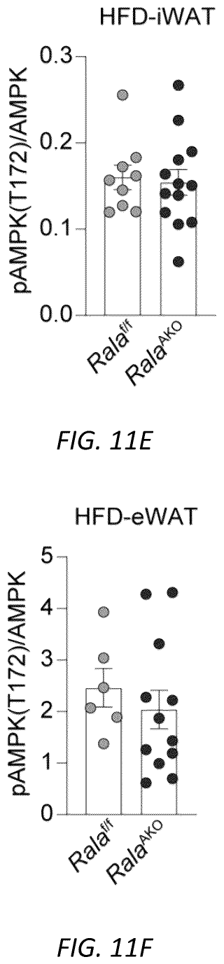

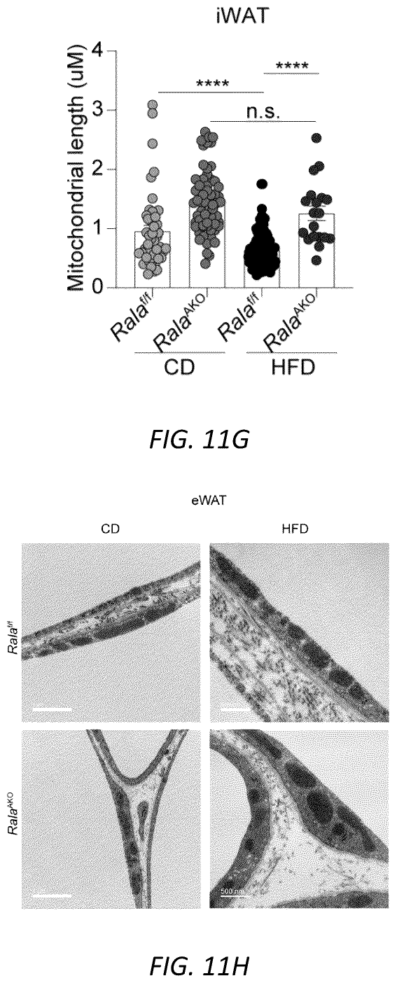

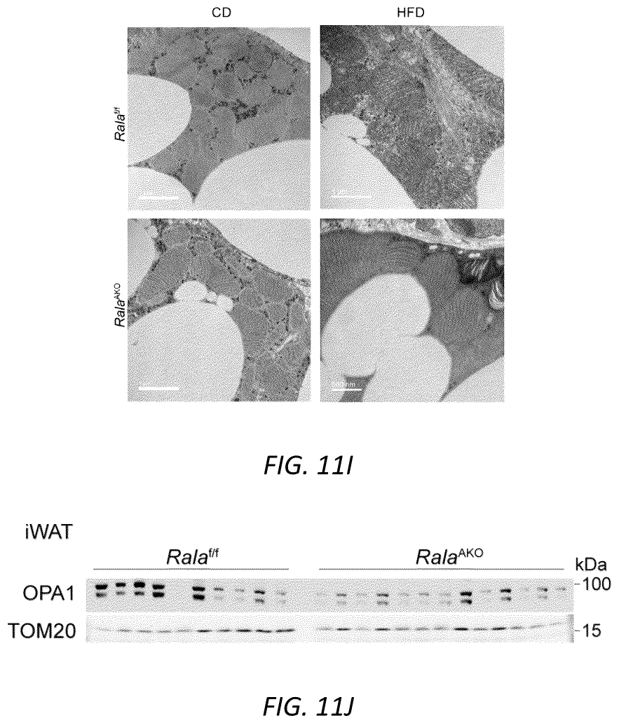

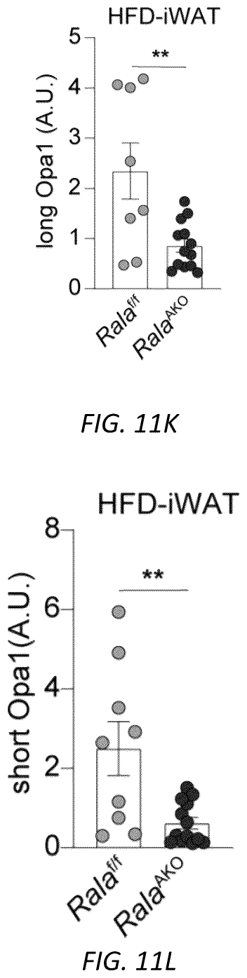

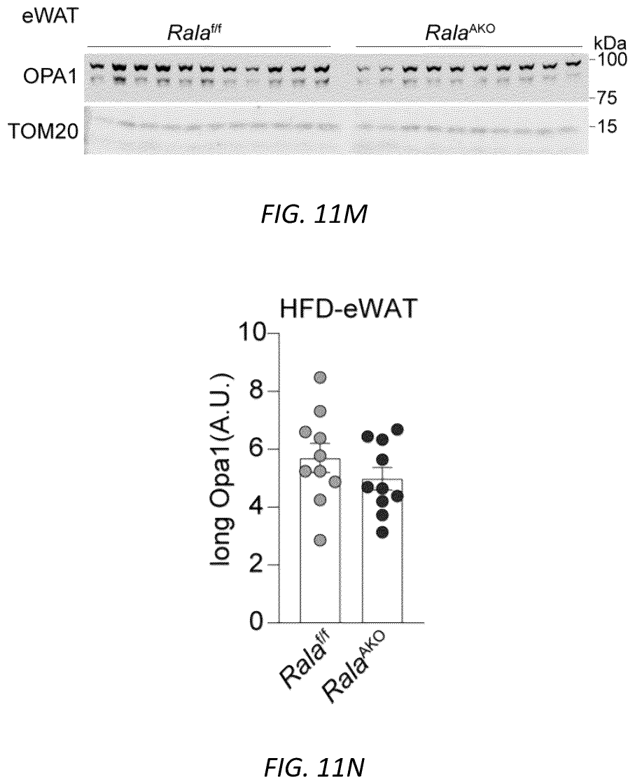

To test if the increased mitochondrial oxidative activity observed in HFD-fed Rala^{AKO} mice was caused by increased mitochondrial biogenesis, expression of genes related to mitochondrial biogenesis was analyzed. It was found that the expression of genes related to mitochondrial biogenesis was comparable between the genotypes (FIGS. 11A and 11B) in WAT. The activity of AMP-activated protein kinase (AMPK), the master regulator of mitochondrial biogenesis^{34,35}, was also comparable between the control and the Rala^{AKO} mice fed with HFD (FIGS. 11C-11F). In addition to biogenesis, mitochondrial function can also be regulated by dynamic changes in morphology through tightly controlled fusion and fission events that shape the organelle to comply with energy demands^{19,36}. Electron microscopy (EM) revealed that HFD feeding of WT mice induced the appearance of smaller and spherical mitochondria in iWAT (FIG. 4D), consistent. It was observed that mitochondria in iWAT changed from an elongated shape in CD-fed mice to a smaller size in HFD-fed mice (FIG. 11G). Consistent with unaltered in vivo metabolic phenotypes, adipocyte Rala deletion did not grossly affect the mitochondrial morphology in iWAT of CD-fed mice. However, the HFD-induced change in mitochondrial morphology was completely prevented in Rala KO iWAT (FIG. 11G). Indeed, tissue weight (FIG. 1G), OXPHOS content (FIGS. 9O and 9P), and mitochondrial OCR (FIG. 3F) were not affected by RalA deletion in eWAT, corresponding to the observation that the appearance of fragmented mitochondria in this depot was not reversed by Rala KO in HFD mice (FIG. 11H). In fact, mitochondria in eWAT did not undergo significant fragmentation in response to HFD, possibly because of their already fragmented shape, consistent with the overall anabolic function of visceral adipocytes³⁹. Moreover, mitochondrial morphology in BAT was not altered by RalA deletion in CD- or HFD-fed mice (FIG. 11I). The mitochondrial morphology was also examined in the immortalized adipocytes differentiated from iWAT. As shown in FIG. 4E, mitochondria in KO adipocytes seemed longer than those in the WT cells. There was a higher frequency of elongated mitochondria (1.0-1.5 μm) in KO cells (FIG. 4F), and the mean maximal mitochondrial length was significantly higher in the WT cells (FIG. 4G).

### Example 7: Inhibition of RalA Increased Drp1 S637 Phosphorylation in White Adipocytes

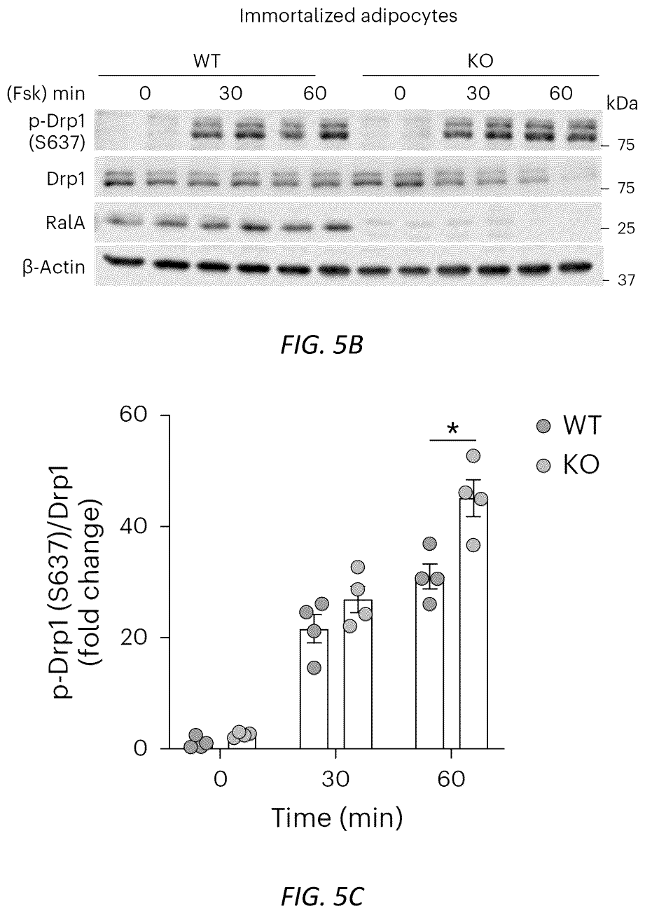

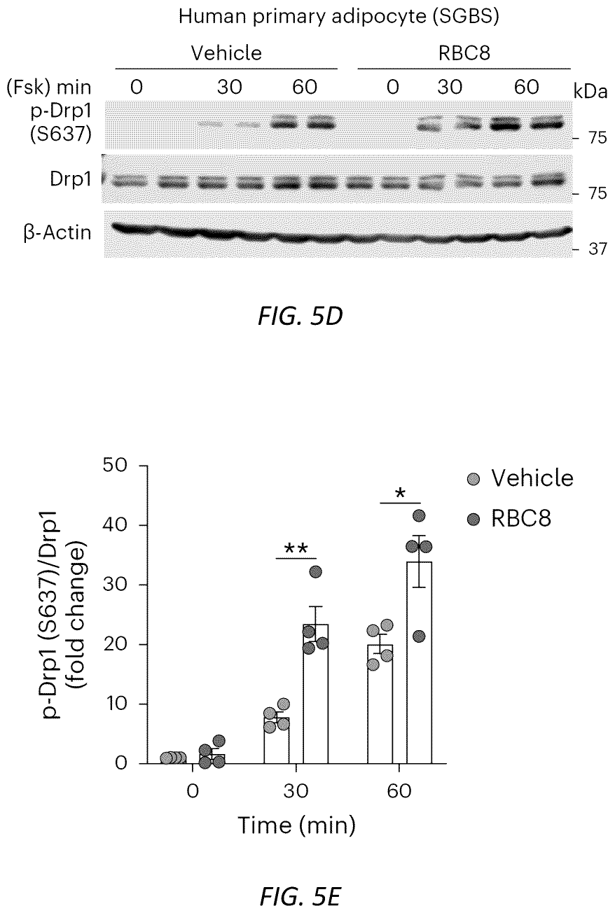

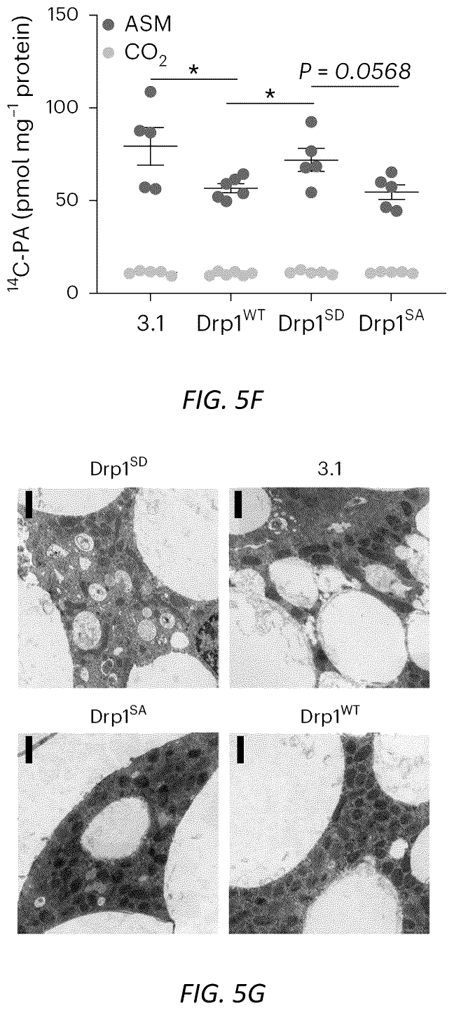

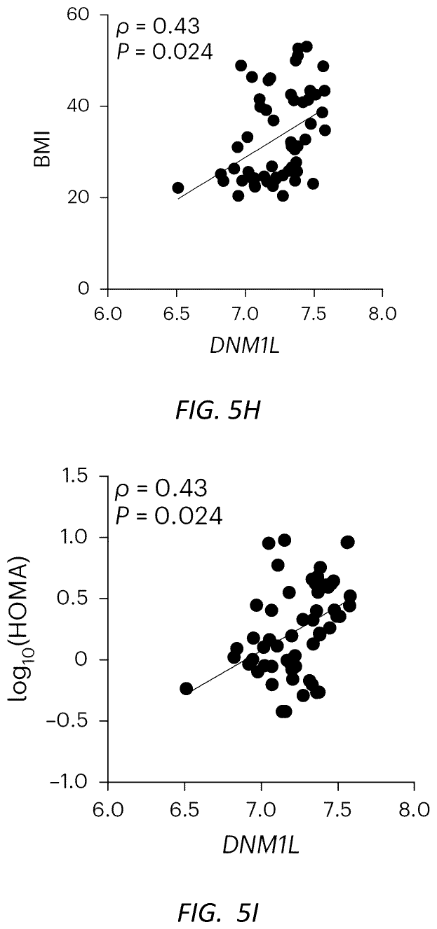

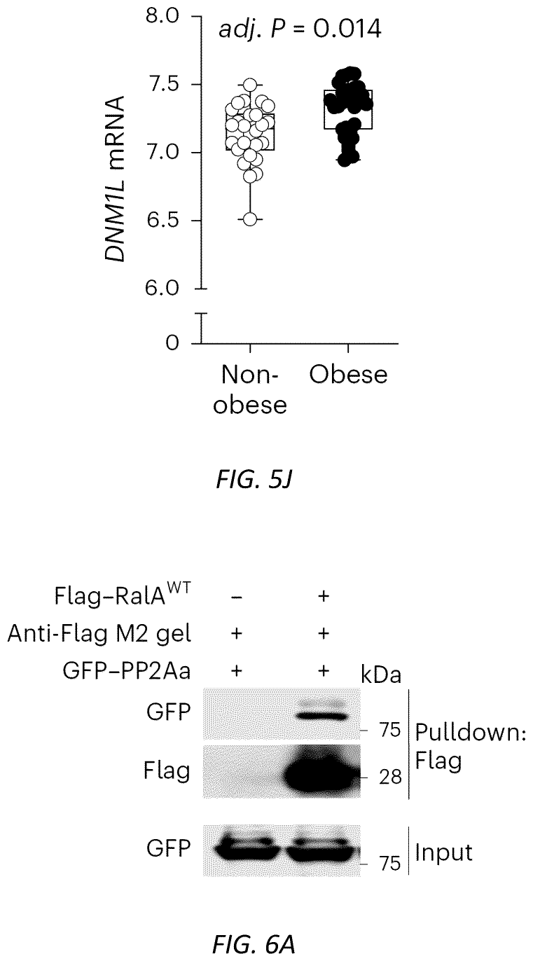

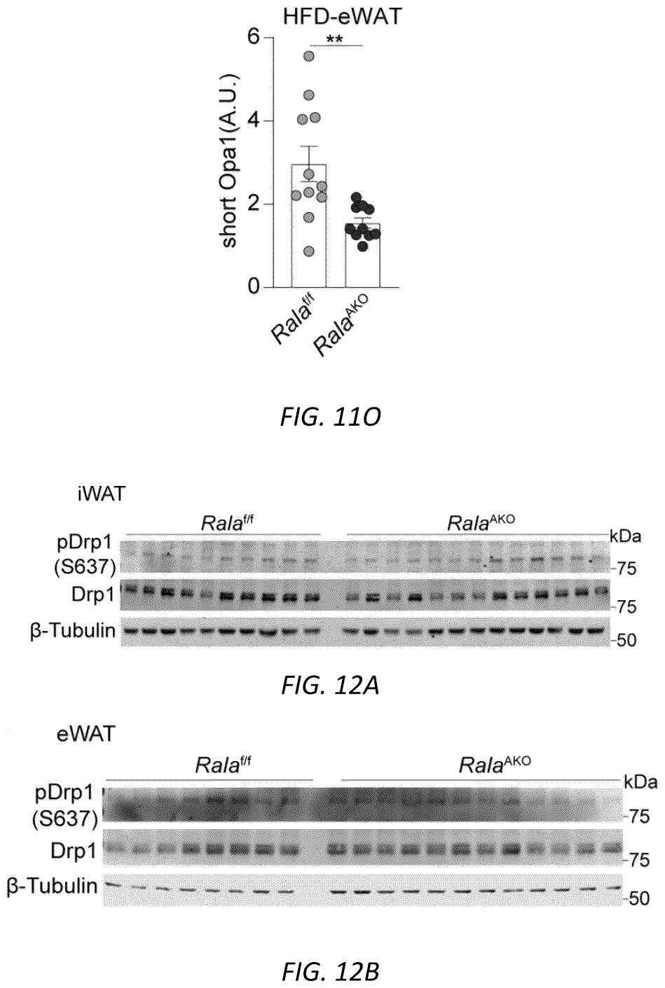

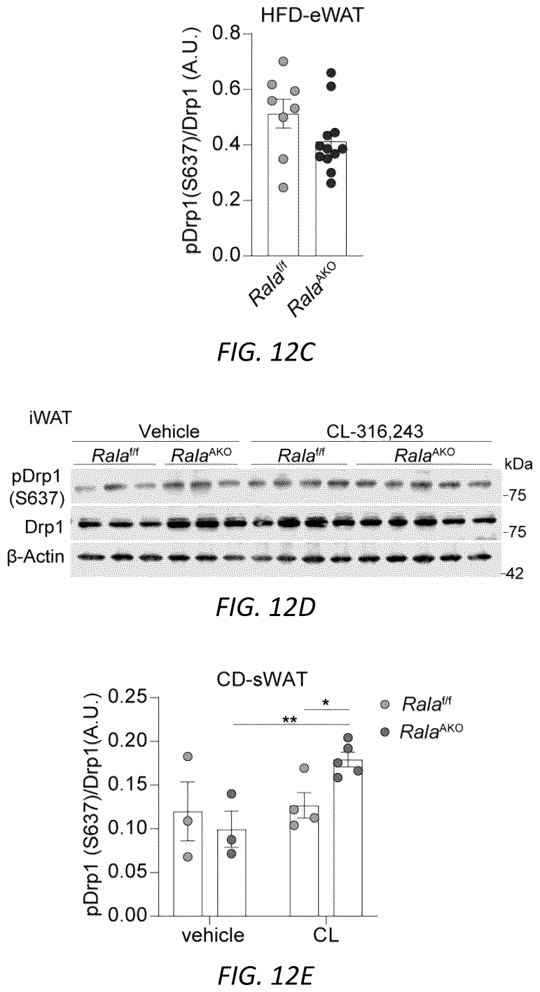

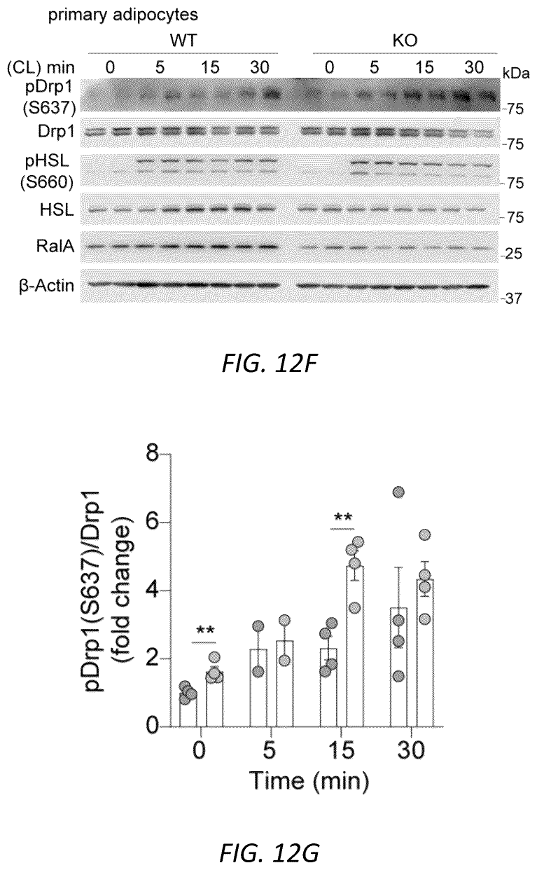

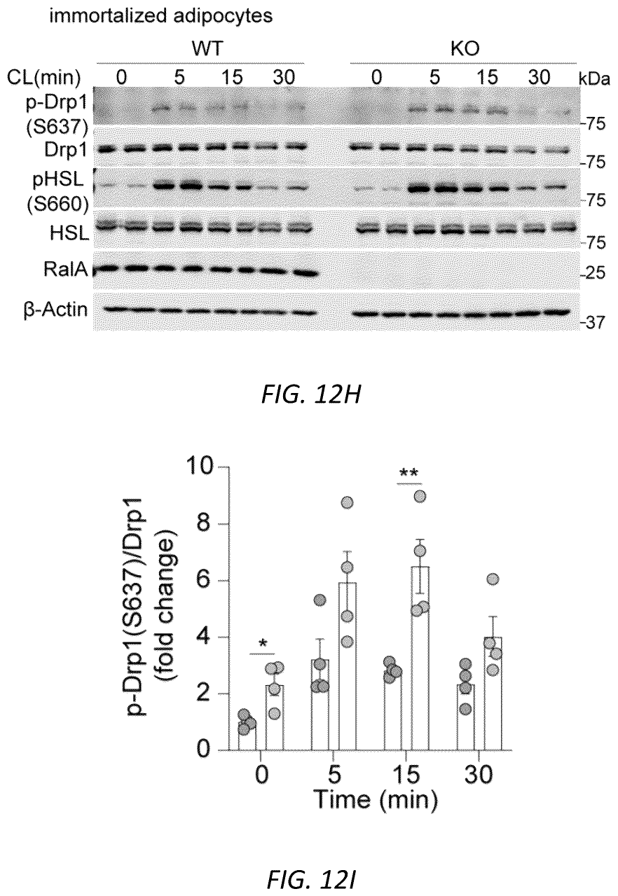

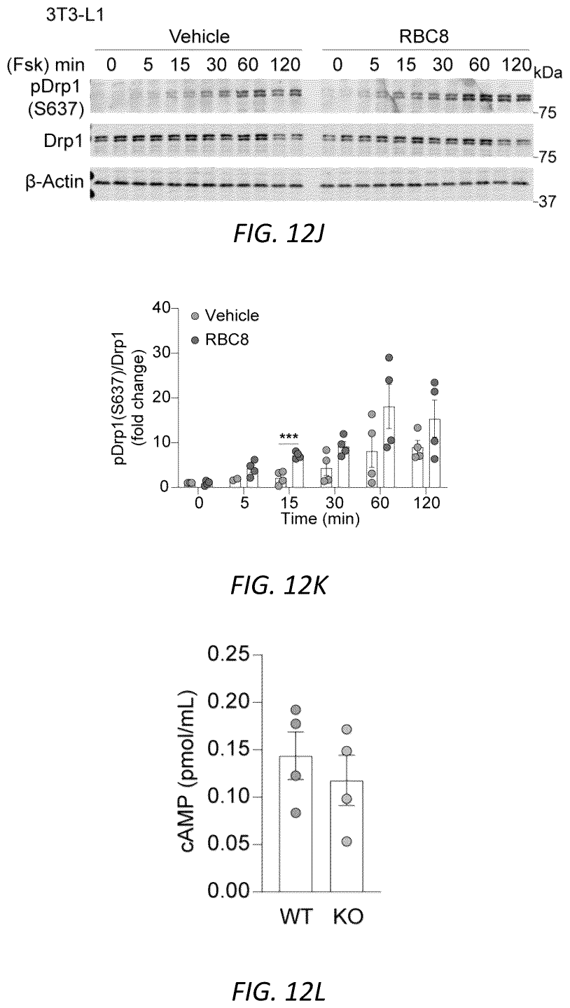

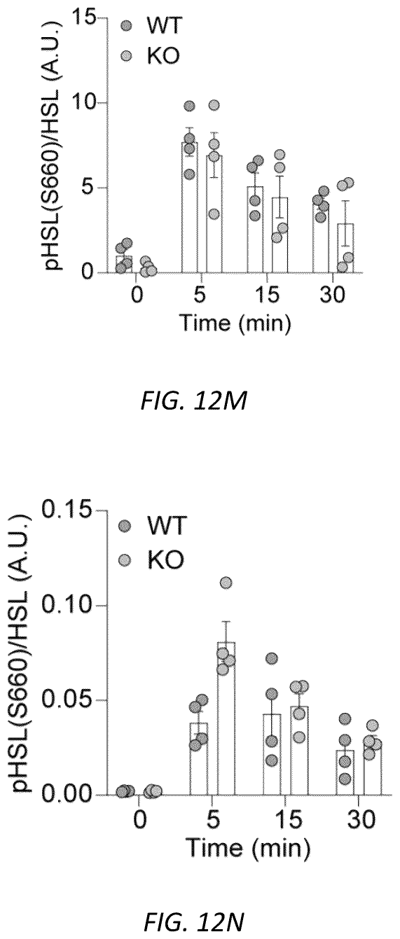

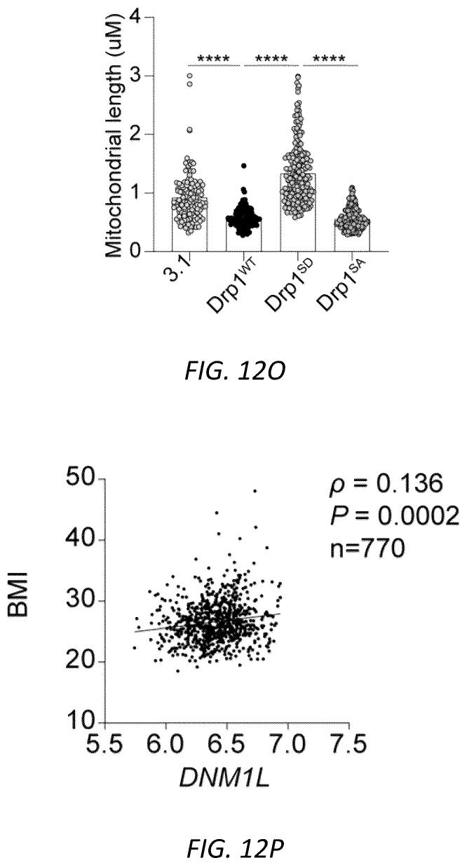

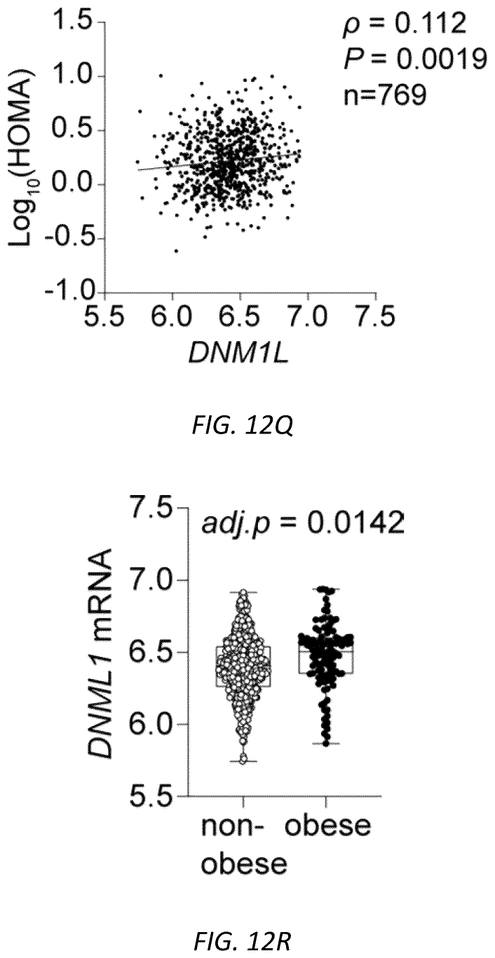

Opa1 and Drp1 are key regulators of mitochondrial fusion and fission, respectively⁴⁰. Opa1 undergoes proteolytic cleavage to generate long (L-Opal) and short (S-Opal) forms that together fuel mitochondrial fusion⁴¹⁻⁴³. Protein levels of both forms of Opa1 were downregulated in iWAT after HFD feeding (FIGS. 11J-11L). Only S-Opa1 was downregulated in eWAT from Rala^{AKO} mice (FIGS. 11M-110), indicating the likelihood of reduced fusion in KO mice compared to the WT littermates. However, the elongated mitochondria in KO mice (FIG. 4D) suggested that this change in Opa1 processing is likely to be compensatory. It was then focused on Drp1 as a key regulator of fission. Notably, Drp1 phosphorylation at the anti-fission S637 site was significantly increased in Rala KO iWAT (FIGS. 5A 12A), whereas Drp1 S637 phosphorylation was comparable between the genotypes in eWAT (FIGS. 12B and 12C). To assess the role of RalA in modulating PKA action at the Drp1 S637 phosphorylation site, the Drp1 S637 phosphorylation was assessed in iWAT. Phosphorylation was higher in CD-fed KO compared to the WT mice in response to B-adrenergic stimulation (FIGS. 12D and 12E). This result ruled out the indirect regulation of Drp1 S637 phosphorylation by body weight differences in HFD-fed mice.

To establish whether this effect is cell-autonomous, Drp1 phosphorylation was examined in both immortalized and primary adipocytes. Consistent with the in vivo results, Rala KO adipocytes showed a significantly higher Drp1 S637 after forskolin and β-adrenergic stimulation compared to WT cells (FIGS. 5B, 5C, and 12F-12I). The effectS of RalA on Drp1 S637 phosphorylation state was also tested using a specific Ral inhibitor that prevents activation and retains GTPase in the GDP-bound, inactive state26,46. Pretreatment with the pan-Ral inhibitor RBC8 significantly increased forskolin-stimulated Drp1 S637 phosphorylation in 3T3-L1 adipocytes (FIGS. 12J and 12K). Inhibition of RalA activity with RBC8 also increased forskolin-stimulated Drp1 S637 phosphorylation in the human primary adipocyte cell line SGBS (FIGS. 5D and 5E). To determine whether RalA influences CL-induced PKA activation or CAMP breakdown, cAMP production and phosphorylation of hormone-sensitive lipase (HSL) were tested in adipocytes. There were no differences in the cAMP production between the WT and the KO primary adipocytes after 5 minutes of CL stimulation (FIG. 12L). Similarly, HSL S660 phosphorylation was identical in the WT and the KO adipocytes (FIGS. 12M and 12N). Thus, RalA specifically modulates Drp1 S637 phosphorylation downstream of PKA activation across multiple adipocyte cell lines of both murine and human origin.

To further investigate the role of Drp1 S637 phosphorylation in mitochondrial oxidative activity and morphology, S637 phospho-mimetic (SD) and phospho-null (SA) mutants were introduced into adipocytes and examined FAO and mitochondrial morphology. Cells expressing Drp1SD had higher FAO than those expressing Drp1WT and Drp1SA (FIG. 5F). Consistent with this result, mitochondrial length in Drp1SD-expressing cells was higher than those with Drp1WT and Drp1SA expression (FIGS. 5G and 12O).

To examine the relevance of Drp1 as a regulator of metabolism in human obesity, the microarray data of abdominal subcutaneous WAT from obese and non-obese women were examined. In human subcutaneous WAT, DNM1L (encoding human Drp1 protein) expression was positively correlated with body mass index (BMI) and HOMA-IR (FIGS. 5H and 51), and its expression was significantly upregulated in obese individuals (FIG. 5J), indicating that the increased expression of DNM1L may contribute to mitochondrial dysfunction in obesity. Moreover, bioinformatic analysis of published microarray data (GEO GSE70353) from 770 human males further confirmed that DNM1L is associated with obesity (FIGS. 12P-12R). Together, these in vivo and in vitro data showed that upregulated Drp1 activity in adipose tissue can contribute to mitochondrial dysfunction during obesity, and further, that RalA deficiency protects mitochondria from excessive fission by increasing Drp1 S637 phosphorylation.

### Example 8: RalA Interacted with Drp1 and Protein Phosphatase 2A, Promoting Dephosphorylation of Drp1 at S637

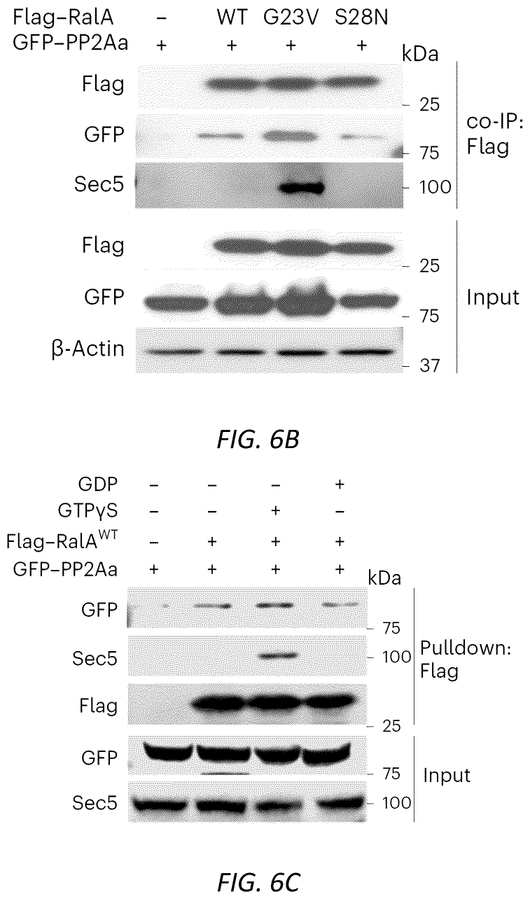

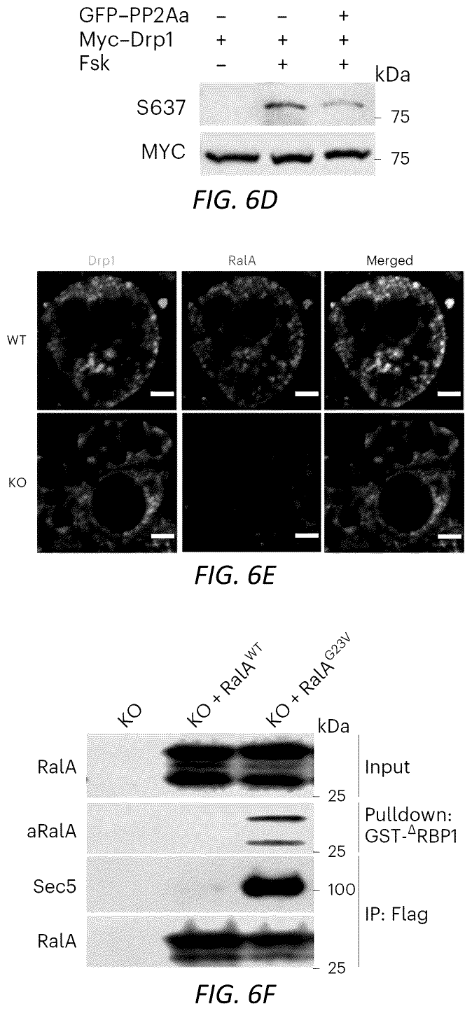

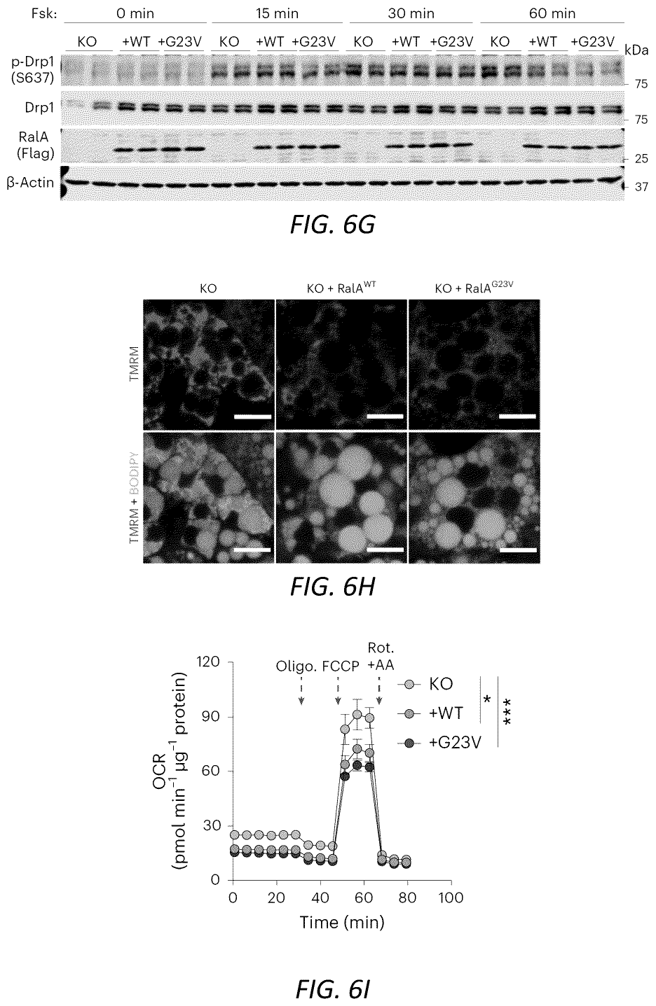

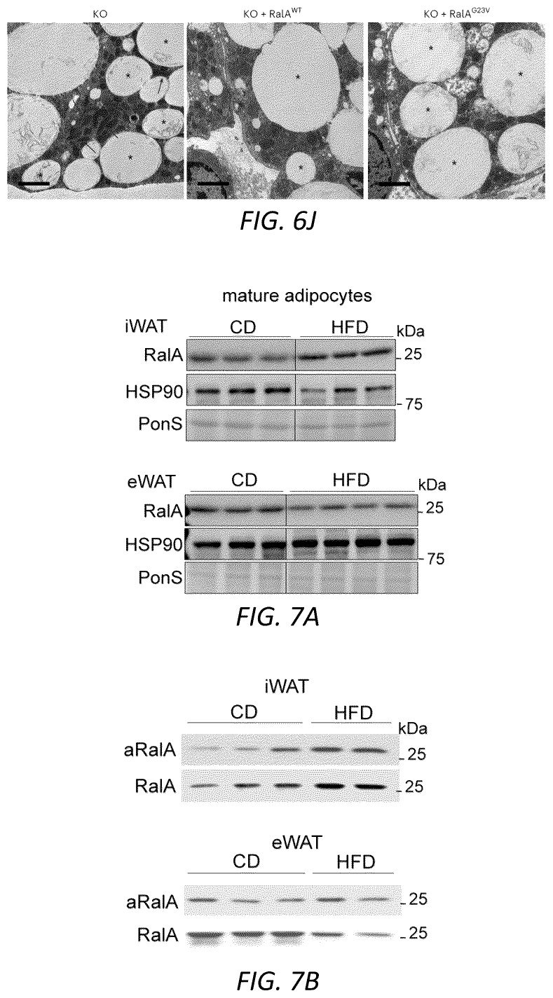

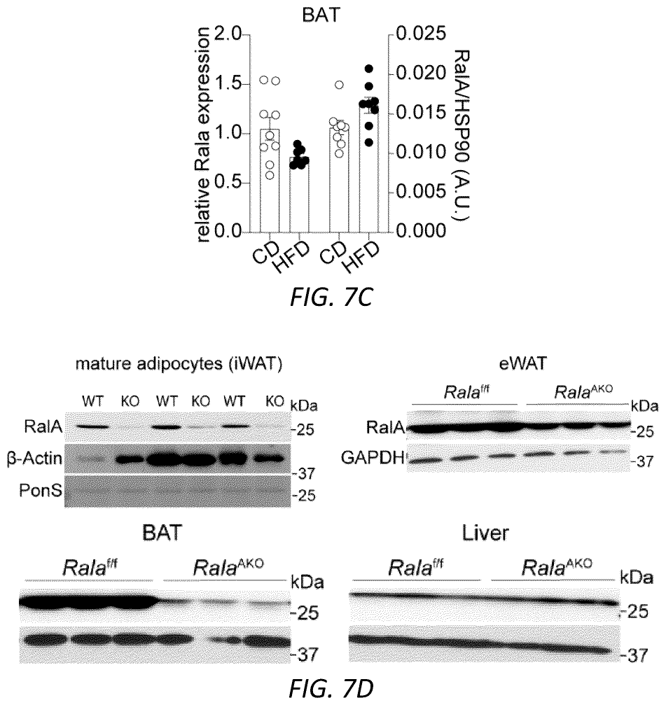

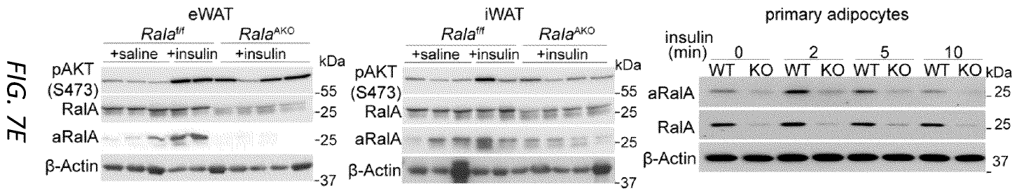

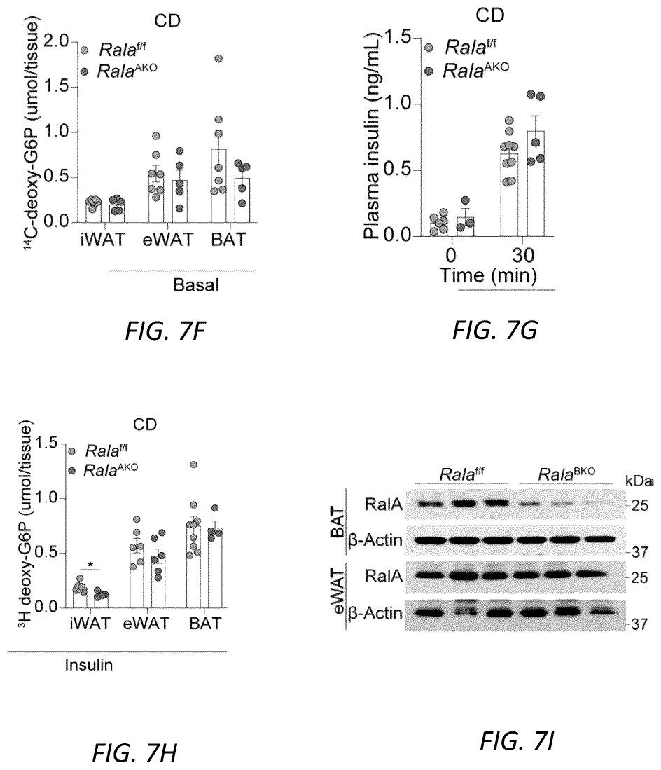

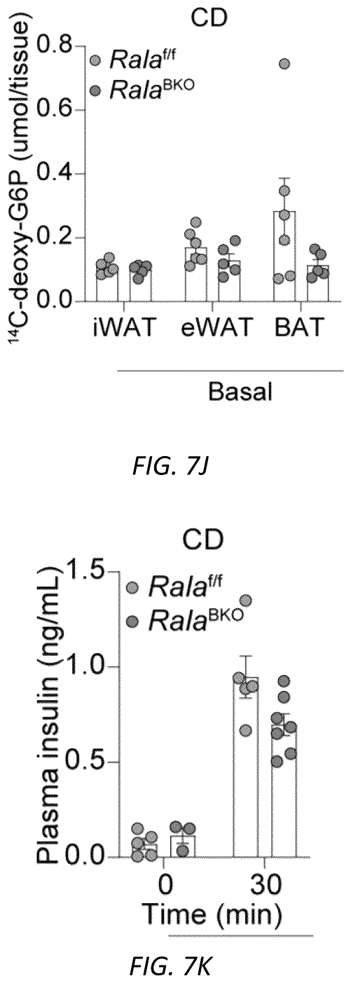

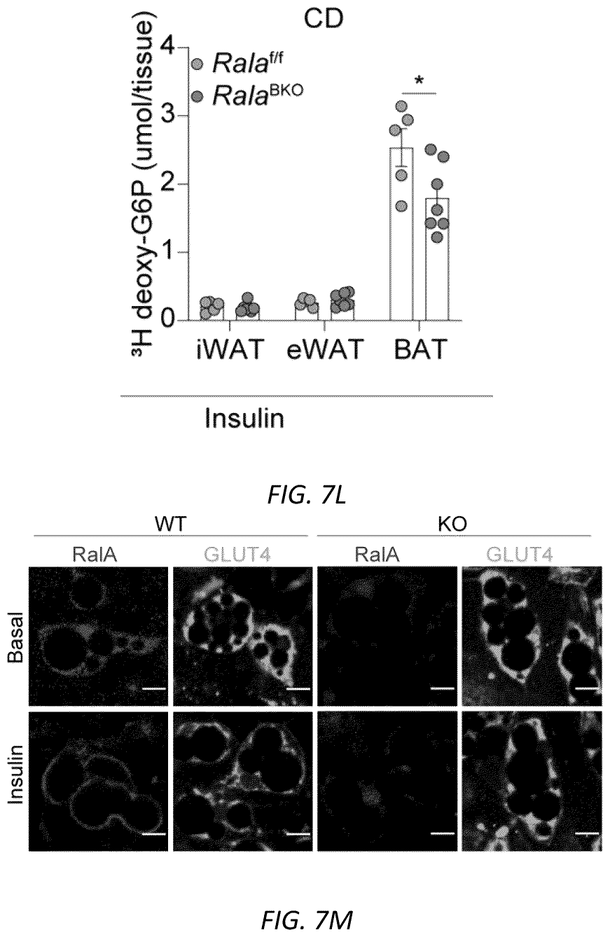

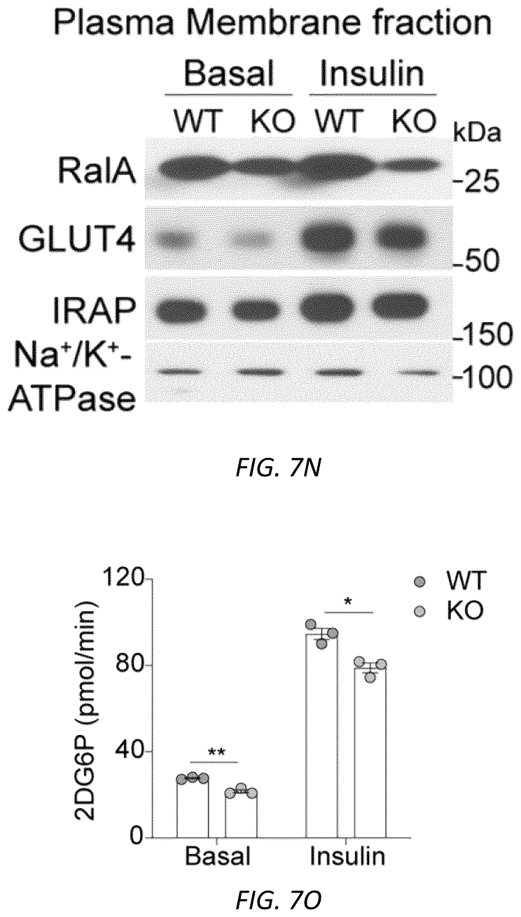

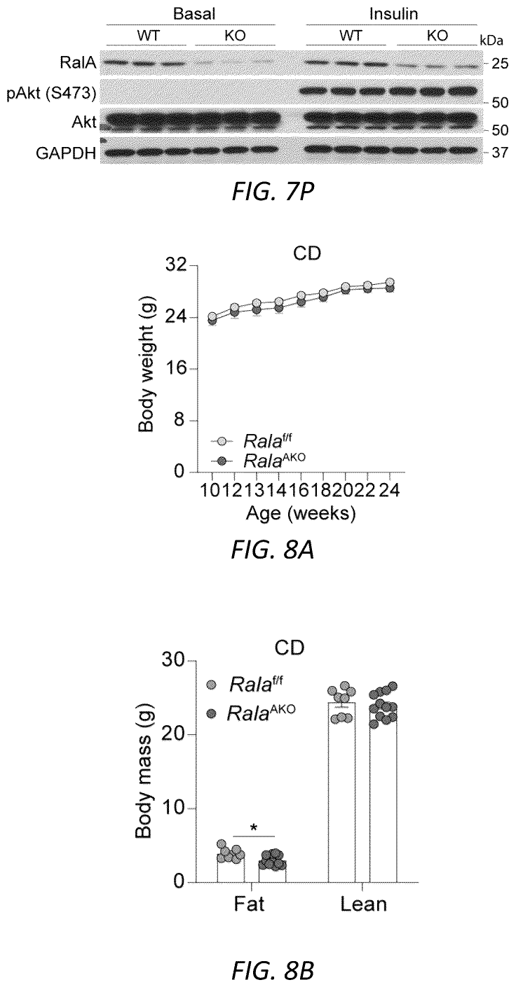

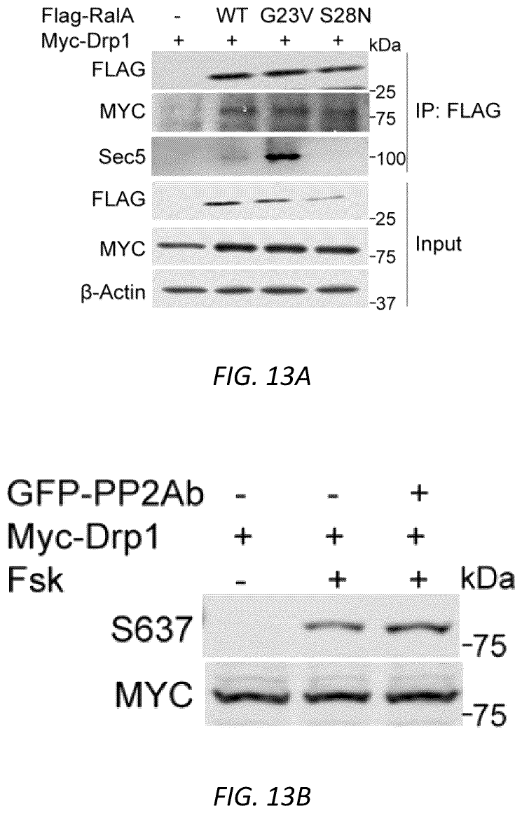

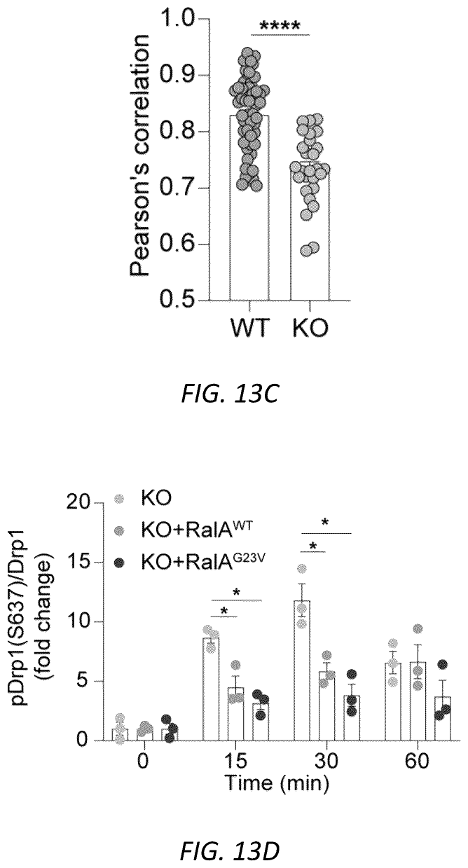

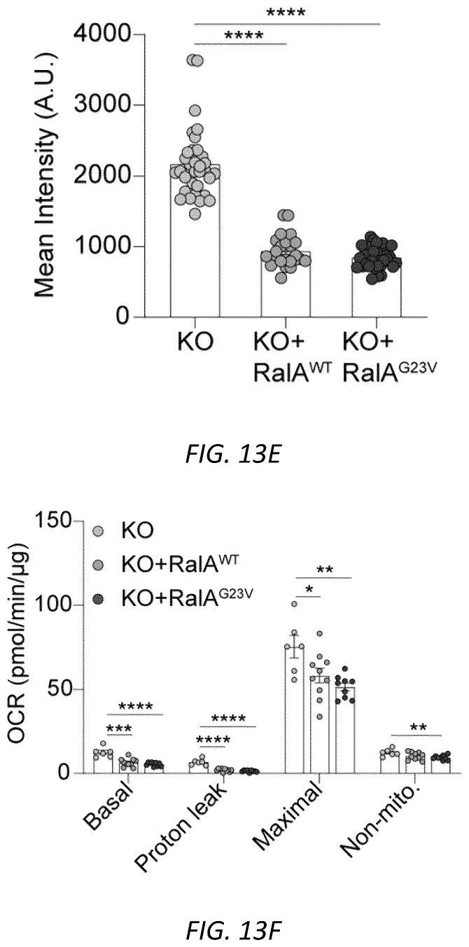

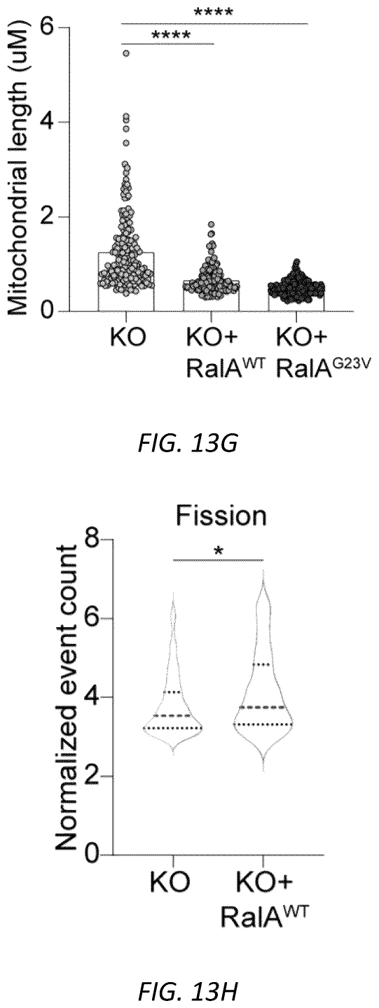

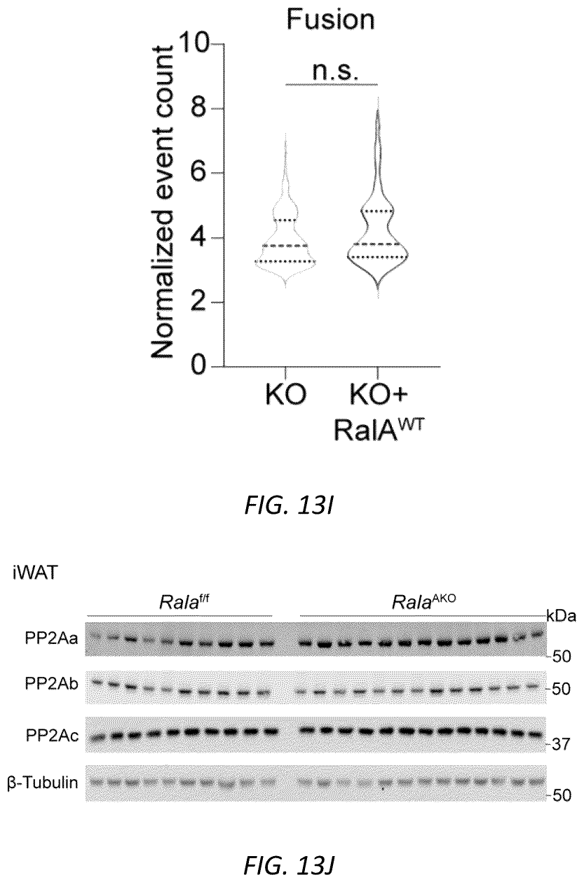

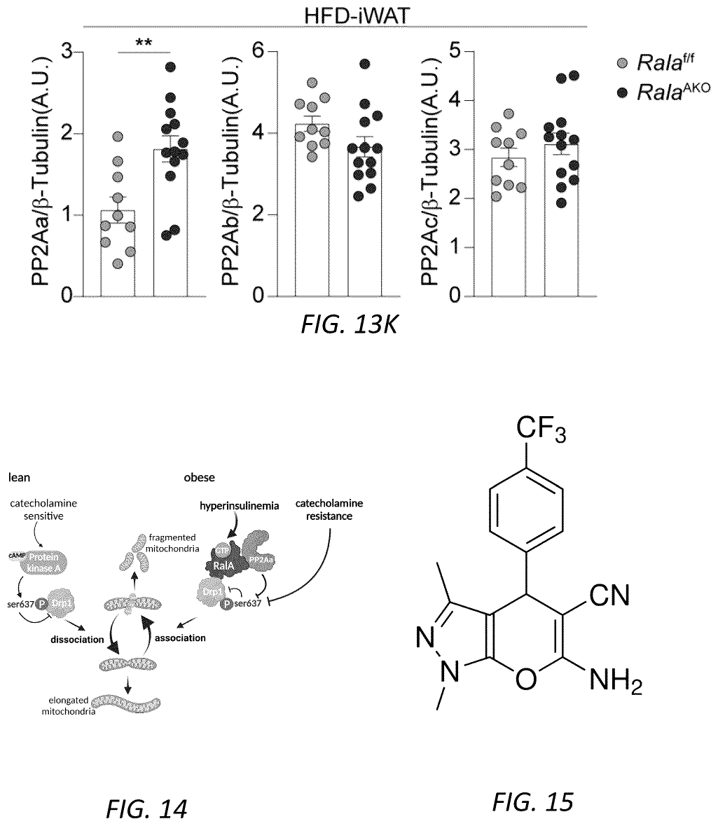

To understand the molecular mechanism by which RalA regulates Drp1 S637 phosphorylation, proteomics was used to search for proteins interacting with WT, constitutively active (G23V) or dominant negative (S28N) forms of RalA ectopically expressed in liver. Among the binding proteins was protein phosphatase 2A subunit Aa (PP2Aa), the scaffolding subunit encoded by the Ppp2r1a gene, which preferentially bound the RalA^{G23V} constitutively active mutant. To confirm these mass spectrometry data, RalA^{WT}-Flag protein was purified from HEK293T cells and pulled down PP2Aa from the lysate (FIG. 6A). To determine whether this interaction is dependent on the activation state of the G protein, we coexpressed WT and mutant RalA constructs with PP2Aa in HEK293T cells. As a positive control, the effector Sec5 only bound to active RalA^{G23V} (ref. 47). Similarly, this mutant form of RalA had the highest affinity for PP2Aa (FIG. 6B). An RalA-Flag fusion protein was also loaded in vitro with GTPγS or GDP to evaluate the specificity of effector binding²². Both Sec5 and PP2Aa were pulled down by RalA loaded with GTPγS but not with GDP (FIG. 6C). In addition, because PP2Aa and Drp1 did not independently interact, it was investigated whether RalA directly modifies Drp1 phosphorylation via PP2Aa. When coexpressed, Drp1 and RalA interacted directly with each other, although there was no preference for the activation state of RalA (FIG. 13A). Activation of the CAMP-PKA axis by addition of forskolin increased Drp1 S637 phosphorylation, whereas the coexpression of PP2Aa promoted the dephosphorylation of S637 (FIG. 6D), although the overexpression of PP2Ab had no effect (FIG. 13B). These data suggested that Drp1 is constitutively associated with RalA independent of activation state; and upon activation, RalA recruits PP2Aa to promote the dephosphorylation of Drp1 S637.

Drp1 colocalized with RalA in adipocytes and this colocalization was not observed in RalA KO adipocytes (FIGS. 6E and 13C). To further examine the effects of RalA activation state on Drp1 phosphorylation and mitochondrial function, immortalized RalA KO cells were transduced with RalA^{WT} and RalA^{G23V}. lentivirus before differentiating into adipocytes. RalA^{G23V}-expressing adipocytes showed a robust increase in RalA-GTP binding (FIG. 6F). These cells had significantly less Drp1 S637 phosphorylation (FIGS. 6G and 13D). Expression of either RalA^{WT} or RalA^{G23V} significantly reduced mitochondrial potential in KO adipocytes (FIGS. 6H and 13E). To confirm that this reduction in mitochondrial potential is associated with reduced oxidative function, a sea-horse assay was performed. Consistent with the results observed in the primary adipocytes, RalA^{WT}- and RalA^{G23V}-expressing adipocytes showed reduced basal and maximal OCR in comparison to the KO adipocytes (FIGS. 6I and 13F). In addition, EM revealed that overexpression of WT or constitutively active RalA in adipocytes resulted in fragmented mitochondria, indicating increased fission compared to RalA KO adipocytes (FIGS. 6J and 13G). Live-cell imaging analyses also indicated fewer fission events in the KO compared to the WT adipocytes, whereas no differences were detected in fusion (FIGS. 13H and 13I).

It was found that RalA deficiency resulted in elongated mitochondria in adipocytes, with increased OXPHOS that dramatically impacted whole-body lipid metabolism; however, contrary to study described elsewhere⁴⁸, no interaction was observed between RalBP1 and Drp1. Notably, the total PP2Aa protein levels were increased in Rala KO compared to the control iWAT, without a difference in PP2Ab and PP2Ac content (FIGS. 13J and 13K), suggesting a compensatory pathway. Taken together, these data suggested that obesity drives RalA expression and GTP binding activity, leading to its association with PP2Aa, which in turn recruits the catalytic subunit PP2Ac to dephosphorylate Drp1 S637. It was also noted that catecholamine resistance, an inherent trait of the obese state²⁸, is also expected to lead to reduced PKA-catalyzed S637 phosphorylation. Together, these effects resulted in constitutive mitochondrial translocation of Drp1 and fragmented mitochondria in adipocytes from obese individuals (FIG. 14).

### Example 9: RalA Inhibitor Reduced Body Weight and Blood Glucose Level in Mice

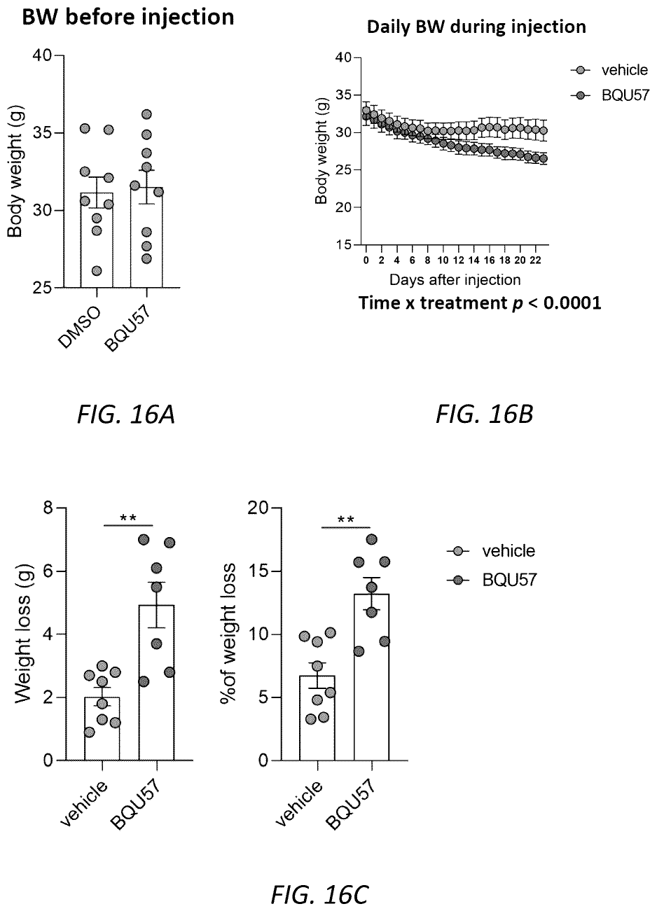

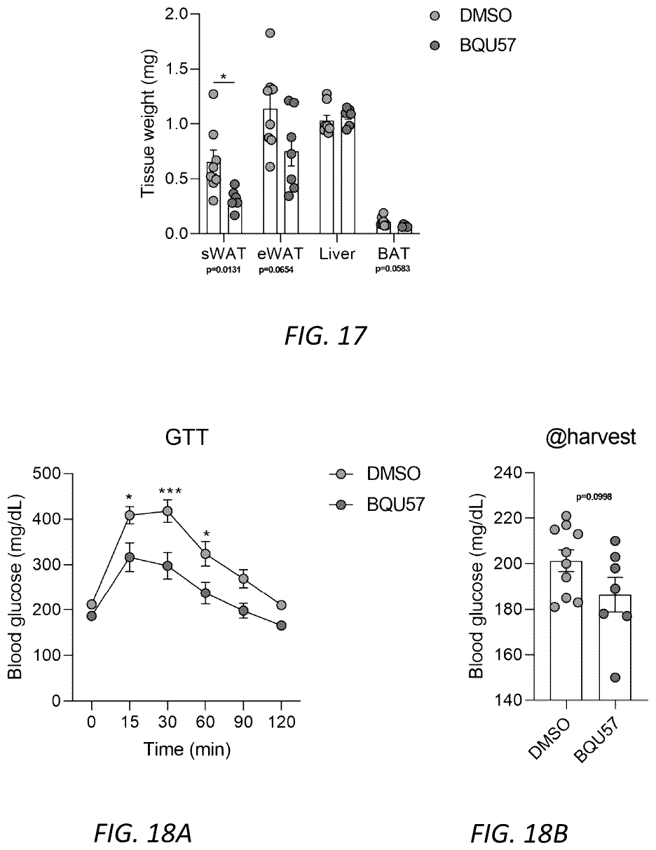

To test the effects of RalA inhibition in reducing body weight, tissue weight and blood glucose level in mice, mice were weighed before injecting with either RalA inhibitor, BQU57, or DMSO (vehicle). Post injection, mice were weighed every 2 days. The results showed a significant weight loss in the BQU57 treated group compared to the vehicle group (FIGS. 16A-16C). Also, the BQU57 treated group showed a marked reduction of subcutaneous WAT (sWAT) weight (FIG. 17). Moreover, it was found that the BQU57 treated group showed a significant reduction in the blood glucose level compared to the vehicle group (FIGS. 18A and 18B).

### REFERENCES

1) Haththotuwa, R. N., Wijeyaratne, C. N., and Senarath, U. Worldwide epidemic of obesity. Obes. Obstet. https://doi.org/10.1016/B978-0-12-817921-5.00001-1 (2020).
2) Saltiel, A. R. Insulin resistance in the defense against obesity. Cell Metab. 15, 798-804 (2012).
3) Saltiel, A. R. & Kahn, C. R. Insulin signalling and the regulation of glucose and lipid metabolism. Nature 414, 799-806 (2001).
4) Kahn, S. E., Hull, R. L. & Utzschneider, K. M. Mechanisms linking obesity to insulin resistance and type 2 diabetes. Nature 444, 840-846 (2006).
5) Reilly, S. M. & Saltiel, A. R. Adapting to obesity with adipose tissue inflammation. Nat. Rev. Endocrinol. 13, 633-643 (2017).
6) Hotamisligil, G. S. Inflammation and metabolic disorders. Nature 444, 860-867 (2006).
7) Kusminski, C. M. & Scherer, P. E. Mitochondrial dysfunction in white adipose tissue. Trends Endocrinol. Metab. 23, 435-443 (2012).
8) Rambold, A. S., Cohen, S. & Lippincott-Schwartz, J. Fatty acid trafficking in starved cells: regulation by lipid droplet lipolysis, autophagy, and mitochondrial fusion dynamics. Dev. Cell 32, 678-692 (2015).
9) Eun, H. K. et al. Essential role of mitochondrial function in adiponectin synthesis in adipocytes. Diabetes 56, 2973-2981 (2007).
10) Tormos, K. V. et al. Mitochondrial complex III ROS regulate adipocyte differentiation. Cell Metab. 14, 537-544 (2011).
11) Mayer, J., Bates, M. W. & Dickie, M. M. Hereditary diabetes in genetically obese mice. Science 113, 746-747 (1951).
12) Kahn, B. B. & Flier, J. S. Obesity and insulin resistance. J. Clin. Invest. 106, 473-481 (2000).
13) Sebastián, D. et al. Mitofusin 2 (Mfn2) links mitochondrial and endoplasmic reticulum function with insulin signaling and is essential for normal glucose homeostasis. Proc. Natl Acad. Sci. USA 109, 5523-5528 (2012).
14) Kelley, D. E., He, J., Menshikova, E. V. & Ritov, V. B. Dysfunction of mitochondria in human skeletal muscle in type 2 diabetes. Diabetes 51, 2944-2950 (2002).
15) Heilbronn, L. K., Seng, K. G., Turner, N., Campbell, L. V. & Chisholm, D. J. Markers of mitochondrial biogenesis and metabolism are lower in overweight and obese insulin-resistant subjects. J. Clin. Endocrinol. Metab. 92, 1467-1473 (2007).
16) Heinonen, S. et al. Impaired mitochondrial biogenesis in adipose tissue in acquired obesity. Diabetes 64, 3135-3145 (2015).
17) Lindinger, P. W. et al. Important mitochondrial proteins in human omental adipose tissue show reduced expression in obesity. J. Proteom. 124, 79-87 (2015).
18) Pafili, K. et al. Mitochondrial respiration is decreased in visceral but not subcutaneous adipose tissue in obese individuals with fatty liver disease. J. Hepatol. 77, 1504-1514 (2022).
19) Wai, T. & Langer, T. Mitochondrial dynamics and metabolic regulation. Trends Endocrinol. Metab. 27, 105-117 (2016).
20) Mishra, P. & Chan, D. C. Metabolic regulation of mitochondrial dynamics. J. Cell Biol. 212, 379-387 (2016).
21) Inoue, M., Chang, L., Hwang, J., Chiang, S. H. & Saltiel, A. R. The exocyst complex is required for targeting of Glut4 to the plasma membrane by insulin. Nature 422, 629-633 (2003).
22) Chen, X. W., Leto, D., Chiang, S. H., Wang, Q. & Saltiel, A. R. Activation of RalA is required for insulin-stimulated Glut4 trafficking to the plasma membrane via the exocyst and the motor protein Myolc. Dev. Cell 13, 391-404 (2007).
23) Nozaki, S., Ueda, S., Takenaka, N., Kataoka, T. & Satoh, T. Role of RalA downstream of Rac1 in insulin-dependent glucose uptake in muscle cells. Cell. Signal. 24, 2111-2117 (2012).
24) Chen, X. W. et al. Exocyst function regulated by effector phosphorylation. Nat. Cell Biol. 13, 580-588 (2011).
25) Karunanithi, S. et al. A Rab10: RalA G protein cascade regulates insulin-stimulated glucose uptake in adipocytes. Mol. Biol. Cell 25, 3059-3069 (2014).
26) Skorobogatko, Y. et al. RalA controls glucose homeostasis by regulating glucose uptake in brown fat. Proc. Natl Acad. Sci. USA 115, 7819-7824 (2018).
27) Chen, Q. et al. Targeting RalGAPα1 in skeletal muscle to simultaneously improve postprandial glucose and lipid control. Sci. Adv. https://doi.org/10.1126/SCIADV.AAV4116 (2019).
28) Valentine, J. M. et al. β3-Adrenergic receptor downregulation leads to adipocyte catecholamine resistance in obesity. J. Clin. Invest. https://doi.org/10.1172/JCI153357 (2022).
29) Townsend, L. K. & Wright, D. C. Looking on the ‘brite’ side exercise-induced browning of white adipose tissue. Pflug. Arch. Eur. J. Physiol. 471, 455-465 (2018).
30) Ouellet, V. et al. Brown adipose tissue oxidative metabolism contributes to energy expenditure during acute cold exposure in humans. J. Clin. Invest. 122, 545-552 (2012).
31) Kajimura, S. & Saito, M. A new era in brown adipose tissue biology: molecular control of brown fat development and energy homeostasis. Annu. Rev. Physiol. 76, 225-249 (2014).
32) Davis, R. J. & Martin, B. R. The effect of β-adrenergic agonists on the membrane potential of fat-cell mitochondria in situ. Biochem. J. 206, 611-618 (1982).
33) Reilly, S. M. et al. Catecholamines suppress fatty acid re-esterification and increase oxidation in white adipocytes via STAT3. Nat. Metab. 2, 620-634 (2020).
34) Herzig, S. & Shaw, R. J. AMPK: guardian of metabolism and mitochondrial homeostasis. Nat. Rev. Mol. Cell Biol. 19, 121-135 (2017).
35) Zhao, P. et al. TBK1 at the crossroads of inflammation and energy homeostasis in adipose tissue. Cell 172, 731-743 (2018).
36) Liesa, M. & Shirihai, O. S. Mitochondrial dynamics in the regulation of nutrient utilization and energy expenditure. Cell Metab. 17, 491-506 (2013).
37) Wilson-Fritch, L. et al. Mitochondrial remodeling in adipose tissue associated with obesity and treatment with rosiglitazone. J. Clin. Invest. 114, 1281-1289 (2004).
38) Choo, H. J. et al. Mitochondria are impaired in the adipocytes of type 2 diabetic mice. Diabetologia 49, 784-791 (2006).
39) Seale, P. et al. Prdm16 determines the thermogenic program of subcutaneous white adipose tissue in mice. J. Clin. Invest. 121, 96-105 (2011).
40) Chan, D. C. Mitochondrial dynamics and its involvement in disease. Annu Rev. Pathol. 15, 235-259 (2020).
41) Song, Z., Chen, H., Fiket, M., Alexander, C. & Chan, D. C. OPAI processing controls mitochondrial fusion and is regulated by mRNA splicing, membrane potential, and YmelL. J. Cell Biol. 178, 749-755 (2007).
42) Mishra, P., Carelli, V., Manfredi, G. & Chan, D. C. Proteolytic cleavage of Opa1 stimulates mitochondrial inner membrane fusion and couples fusion to oxidative phosphorylation. Cell Metab. 19, 630-641 (2014).
43) Ge, Y. et al. Two forms of opal cooperate to complete fusion of the mitochondrial inner-membrane. eLife https://doi.org/10.7554/ELIFE.50973 (2020).
44) Chang, C. R. & Blackstone, C. Cyclic AMP-dependent protein kinase phosphorylation of Drp1 regulates its GTPase activity andmitochondrial morphology. J. Biol. Chem. 282, 21583-21587 (2007).
45) Cribbs, J. T. & Strack, S. Reversible phosphorylation of Drp1 by cyclic AMP-dependent protein kinase and calcineurin regulates mitochondrial fission and cell death. EMBO Rep. 8, 939-944 (2007).
46) Yan, C. et al. Discovery and characterization of small molecules that target the GTPase Ral. Nature 515, 443-447 (2014).
47) Moskalenko, S. et al. The exocyst is a Ral effector complex. Nat. Cell Biol. 4, 66-72 (2002).
48) Kashatus, D. F. et al. RALA and RALBP1 regulate mitochondrial fission at mitosis. Nat. Cell Biol. 13, 1108-1115 (2011).
49) Xiao, Y. X. et al. Adipocyte mitochondrial function is reduced in human obesity independent of fat cell size. J. Clin. Endocrinol. Metab. 99, E209 (2014).
50) Spiegelman, B. M. & Flier, J. S. Obesity and the regulation of energy balance. Cell 104, 531-543 (2001).
51) Chen, Q. et al. GARNL1, a major RalGAP a subunit in skeletal muscle, regulates insulin-stimulated RalA activation and GLUT4 trafficking via interaction with 14-3-3 proteins. Cell. Signal. 26, 1636-1648 (2014).
52) Marette, A. et al. Regional variation in adipose tissue insulin action and GLUT4 glucose transporter expression in severely obese premenopausal women. Diabetologia 40, 590-598 (1997).
53) Carvalho, E., Jansson, P., Nagaev, I., Wenthzel, A. & Smith, U. Insulin resistance with low cellular IRS-1 expression is also associated with low GLUT4 expression and impaired insulin-stimulated glucose transport 1. FASEB J. 15, 1101-1103 (2001).
54) Lumeng, C. N., Deyoung, S. M., and Saltiel, A. R. Macrophages block insulin action in adipocytes by altering expression of signaling and glucose transport proteins. Am. J. Physiol. Endocrinol. Metab. https://doi.org/10.1152/AJPENDO.00284.2006 (2007).
55) Trayhurn, P., Wang, B. & Wood, I. S. Hypoxia in adipose tissue: a basis for the dysregulation of tissue function in obesity? Br. J. Nutr. 100, 227-235 (2008).
56) Rosen, E. D. & Spiegelman, B. M. Adipocytes as regulators of energy balance and glucose homeostasis. Nature 444, 847-853 (2006).
57) Perry, R. J. et al. Hepatic acetyl COA links adipose tissue inflammation to hepatic insulin resistance and type 2 diabetes. Cell 160, 745-758 (2015).
58) Giralt, M. & Villarroya, F. White, brown, beige/brite: different adipose cells for different functions? Endocrinology 154, 2992-3000 (2013).
59) Bartelt, A. & Heeren, J. Adipose tissue browning and metabolic health. Nat. Rev. Endocrinol. 10, 24-36 (2013).
60) Forner, F. et al. Proteome differences between brown and white fat mitochondria reveal specialized metabolic functions. Cell Metab. 10, 324-335 (2009).
61) Banerjee, R., Mukherjee, A. & Nagotu, S. Mitochondrial dynamics and its impact on human health and diseases: inside the DRP1 blackbox. J. Mol. Med. 100, 1-21 (2021).
62) Jheng, H.-F. et al. Mitochondrial fission contributes to mitochondrial dysfunction and insulin resistance in skeletal muscle. Mol. Cell. Biol. 32, 309-319 (2012).
63) Kugler, B. A. et al. Roux-en-Y gastric bypass surgery restores insulin-mediated glucose partitioning and mitochondrial dynamics in primary myotubes from severely obese humans. Int. J. Obes. 44, 684-696 (2019).
64) Gundersen, A. E. et al. Altered mitochondrial network morphology and regulatory proteins in mitochondrial quality control in myotubes from severely obese humans with or without type 2 diabetes. Appl. Physiol. Nutr. Metab. 45, 283-293 (2020).
65) Wikstrom, J. D. et al. Hormone-induced mitochondrial fission is utilized by brown adipocytes as an amplification pathway for energy expenditure. EMBO J. 33, 418 (2014).
66) Li, X. et al. Novel role of dynamin-related-protein 1 in dynamics of ER-lipid droplets in adipose tissue. FASEB J. 34, 8265 (2020).
67) Kugler, B. A. et al. Pharmacological inhibition of dynamin-related protein 1 attenuates skeletal muscle insulin resistance in obesity. Physiol. Rep. 9, e14808 (2021).
68) Finocchietto, P. et al. Inhibition of mitochondrial fission by Drp-1 blockade by short-term leptin and Mdivi-1 treatment improves white adipose tissue abnormalities in obesity and diabetes. Pharmacol. Res. 178, 106028 (2022)
69) Wang, L. et al. Disruption of mitochondrial fission in the liver protects mice from diet-induced obesity and metabolic deterioration. Diabetologia 58, 2371-2380 (2015).
70) Mooli, R. G. R., Mukhi, D., Chen, Z., Buckner, N. & Ramakrishnan, S. K. An indispensable role for dynamin-related protein 1 in beige and brown adipogenesis. J. Cell Sci. https://doi.org/10.1242/jcs.247593 (2020).
71) Valera-Alberni, M. et al. Crosstalk between Drp1 phosphorylation sites during mitochondrial remodeling and their impact on metabolic adaptation. Cell Rep. https://doi.org/10.1016/J. CELREP.2021.109565 (2021).
72) Kleele, T. et al. Distinct fission signatures predict mitochondrial degradation or biogenesis. Nature 593, 435-439 (2021).
73) Adachi, Y. et al. Drp1 tubulates the ER in a GTPase-Independent manner. Mol. Cell 80, 621-632 (2020).
74) Czech, M. P. Insulin action and resistance in obesity and type 2 diabetes. Nat. Med. 23, 804-814 (2017).
75) Huang, J. et al. NBR1 is a critical step in the repression of thermogenesis of p62-deficient adipocytes through PPARγ. Nat. Commun. 12, 2876 (2021).
76) Schweiger, M. et al. Pharmacological inhibition of adipose triglyceride lipase corrects high-fat diet-induced insulin resistance and hepatosteatosis in mice. Nat. Commun. 8, 14859 (2017).
77) Arner, E. et al. Adipose tissue MicroRNAs as regulators of CCL2 production in human obesity. Diabetes 61, 1986-1993 (2012).
78) Civelek, M. et al. Genetic regulation of adipose gene expression and cardio-metabolic traits. Am. J. Hum. Genet. 100, 428-443 (2017).
79) Matthews, D. R. et al. Homeostasis model assessment: insulin resistance and β-cell function from fasting plasma glucose and insulin concentrations in man. Diabetologia 28, 412-419 (1985).
80) Mina, A. I. et al. CalR: A web-based analysis tool for indirect calorimetry experiments. Cell Metab. 28, 656-666 (2018).
81) Chen, B. C. et al. Lattice light-sheet microscopy: Imaging molecules to embryos at high spatiotemporal resolution. Science 346, 1257998 (2014).
82) Wang, Z. et al. MitoTNT: mitochondrial temporal network tracking for 4D live-cell fluorescence microscopy data. PLOS Comput. Biol. 19, 1-24 (2023).

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A method of treating or reducing the risk of obesity or a condition associated with obesity in a subject in need thereof, the method comprising administering a therapeutically effective amount of a pharmaceutical composition comprising an RalA inhibitor.

2. A method of treating obesity or a condition associated with obesity in a subject in need thereof, the method comprising:
a) identifying the subject as having hyperinsulinemia, type 2 diabetes, a BMI of 30 or greater, a waist-hip ratio (WHR) of greater than 1.0, prediabetes, and/or fatty liver disease,
b) administering a therapeutically effective amount of a pharmaceutical composition comprising an RalA inhibitor.

3. The method of claim 2, further comprising administering to the subject a second pharmaceutical composition.

4. The method of claim 3, wherein the second pharmaceutical composition is administered before, after, or concurrent with the pharmaceutical composition comprising the RalA inhibitor.

5. The method of claim 4, wherein concurrent administration of the pharmaceutical composition comprising the RalA inhibitor, and the second composition is administered as a single composition.

6. The method of claim 3, wherein the second pharmaceutical composition comprises naltrexone-bupropion, phentermine-topiramate, orlistat, diethylpropion, setmelanotide, phendimetrazine, benzphetamine, tirzepatide, a glucagon-like peptide-1 receptor (GLP-1) agonist, a glucose-dependent insulinotropic polypeptide (GIP), a GIP antagonist, an amylin agonist, a leptin agonist, and/or a glucagon agonist.

7. The method of claim 6, wherein the GLP-1 agonist comprises dulaglutide, exenatide, liraglutide, lixisenatide, and/or semaglutide.

8. The method of claim 6, wherein the pharmaceutical composition or the second pharmaceutical composition comprises tirzepatide.

9. The method of claim 6, wherein the amylin agonist comprises pramlintide.

10. The method of claim 6, wherein the leptin agonist comprises Metreleptin.

11. The method of claim 6, wherein the glucagon agonist comprises dasiglucagon, Baqsimi, or Gvoke.

12. The method of claim 2, wherein the condition associated with obesity comprises type 2 diabetes, hyperinsulinemia, hepatic steatosis, weight gain, glucose intolerance, heart disease, chronic kidney disease, high cholesterol, gall bladder disease, high blood pressure, sleep apnea, gastroesophageal reflex disease, metabolic syndrome, acute pancreatitis, dyslipidemia, and/or cancer.

13. The method of any one of claim 2, wherein the RalA inhibitor is an inhibitory nucleic acid molecule, a CRISPR/Cas system, or a small molecule inhibitor.

14. The method of claim 2, wherein the RalA inhibitor comprises 6-Amino-1,3-dimethyl-4-(4-(trifluoromethyl)phenyl)-1,4-dihydropyrano[2,3-c]pyrazole-5-carbonitrile, SCH-53239, SCH-54292, BQU57, RBC6, RBC8, and/or RBC10.

15. The method of claim 2, wherein the administering comprises oral, intravenous, intradermal, intramuscular, and/or subcutaneous administration.

16. The method of claim 2, wherein the subject is a mammal.

17. The method of claim 16, wherein the mammal is a human, a monkey, a dog, a cat, a pig, a horse, a cow, a sheep, a goat, a rabbit, a mouse, or a rat.
